(19) United States
(12) Patent Application Publication
(10) Pub. No.: US 2012/0065066 A1
(43) Pub. Date: Mar. 15, 2012

(54) NOVEL HERBICIDES

(75) Inventors: Christopher John Mathews, Bracknell (GB); Louisa Robinson, Bracknell (GB); Stephane Andre Marie Jeanmart, Bracknell (GB)

(73) Assignee: SYNGENTA CROP PROTECTION LLC, Greensboro, NC (US)

(21) Appl. No.: 13/320,716

(22) PCT Filed: May 16, 2009
(86) PCT No.: PCT/EP2009/003508

§ 371 Date: Nov. 15, 2011

(51) Int. Cl. A01N 35/06; C07C 251/44; C07C 49/753; C07D 333/28; C07D 213/61; C07D 241/12; C07D 237/08; C07D 239/26; A01N 35/10; A01N 43/10; A01N 43/40; A01N 43/60; A01N 43/58; A01N 43/54; A61P 13/02; C07C 49/747

(52) U.S. Cl. 504/103; 568/326; 564/257; 549/78; 546/285; 544/336; 544/224; 544/294; 504/348; 504/118; 504/344; 504/148; 504/289; 504/129; 504/244; 504/130; 504/235; 504/136; 504/236; 504/137; 504/239

(57) ABSTRACT

Compounds of formula (I), wherein the substituents are as defined in claim 1, are suitable for use as herbicides.

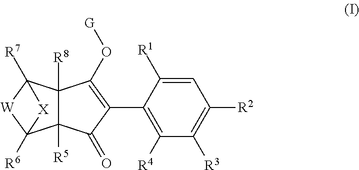

The present invention relates to novel, herbicidally active cyclopentanedione compounds, and derivatives thereof, to processes for their preparation, to compositions comprising those compounds, and to their use in controlling weeds, especially in crops of useful plants, or in inhibiting undesired plant growth.

Cyclopentanedione compounds having herbicidal action are described, for example, in WO 99/48869.

Novel cyclopentanedione compounds, and derivatives thereof, having herbicidal and growth-inhibiting properties have now been found.

The present invention accordingly relates to compounds of formula I

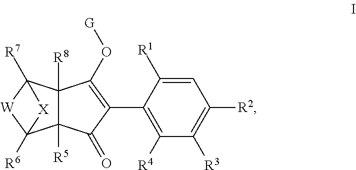

wherein

R¹ is methyl, ethyl, n-propyl, isopropyl, cyclopropyl, halomethyl, haloethyl, vinyl, ethynyl, halogen, methoxy, ethoxy, halomethoxy or haloethoxy,

R² and R³ are independently of each other hydrogen, methyl, ethyl, n-propyl, isopropyl, cyclopropyl, halomethyl, haloethyl, vinyl, propenyl, ethynyl, propynyl, halogen, methoxy, ethoxy, halomethoxy or haloethoxy, optionally substituted aryl or optionally substituted heteroaryl,

R⁴ is hydrogen, methyl, ethyl, n-propyl, isopropyl, cyclopropyl, halomethyl, haloethyl, vinyl, propenyl, ethynyl, propynyl, halogen, methoxy, ethoxy, halomethoxy or haloethoxy,

R⁵ and R⁸ are independently of each other hydrogen, C₁-C₃alkyl, C₁-C₃haloalkyl, C₁-C₃alkoxy, C₁-C₃alkylthio, halogen or C₁-C₆alkoxycarbonyl, or

R⁵ and R⁸ join together to form a 3-7 membered carbocyclic or heterocyclic ring containing an oxygen or sulfur atom,

R⁶ and R⁷ are independently of each other hydrogen, halogen, cyano, hydroxy, optionally substituted C₁-C₆alkyl, optionally substituted C₂-C₆alkenyl, optionally substituted C₂-C₇alkynyl, optionally substituted C₁-C₆alkoxy or tri-C₁-C₄alkylsilyloxy,

X is optionally substituted C₁-C₃alkylene,

W is optionally substituted C₁-C₃alkylene or optionally substituted C₂-C₃alkenylene and

G is hydrogen or an agriculturally acceptable metal, sulfonium, ammonium or latentiating group.

In the substituent definitions of the compounds of the formula I, each alkyl moiety either alone or as part of a larger group (such as alkoxy, alkylthio, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, isopropyl, n-butyl, sec-butyl, isobutyl, tert-butyl or neopentyl. The alkyl groups are suitably C₁-C₆alkyl groups, but are preferably C₁-C₄alkyl or C₁-C₃alkyl groups, and, more preferably, C₁-C₂alkyl groups.

When present, the optional substituents on an alkyl moiety (alone or as part of a larger group such as alkoxy, alkoxycarbonyl, alkylcarbonyl, alkylaminocarbonyl, dialkylaminocarbonyl) include one or more of halogen, nitro, cyano, C₃-C₇cycloalkyl (itself optionally substituted with C₁-C₆alkyl or halogen), C₅-C₇cycloalkenyl (itself optionally substituted with C₁-C₄alkyl or halogen), hydroxy, C₁-C₁₀alkoxy, C₁-C₁₀alkoxy(C₁-C₁₀)alkoxy, tri(C₁-C₄)alkylsilyl(C₁-C₆)alkoxy, C₁-C₆alkoxy-carbonyl(C₁-C₁₀)alkoxy, C₁-C₁₀haloalkoxy, aryl(C₁-C₄)alkoxy (where the aryl group is optionally substituted), C₃-C₇cycloalkyloxy (where the cycloalkyl group is optionally substituted with C₁-C₆alkyl or halogen), C₃-C₁₀alkenyloxy, C₃-C₁₀alkynyloxy, mercapto, C₁-C₁₀alkylthio, C₁-C₁₀haloalkylthio, aryl(C₁-C₄)alkylthio (where the aryl group is optionally substituted), C₃-C₇cycloalkylthio (where the cycloalkyl group is optionally substituted with C₁-C₆alkyl or halogen), tri(C₁-C₄)alkylsilyl(C₁-C₆)alkylthio, arylthio (where the aryl group is optionally substituted), C₁-C₆alkylsulfonyl, C₁-C₆haloalkylsulfonyl, C₁-C₆alkylsulfinyl, C₁-C₆haloalkylsulfinyl, arylsulfonyl (where the aryl group is optionally substituted), tri(C₁-C₄)alkylsilyl, aryldi(C₁-C₄)alkylsilyl, (C₁-C₄)alkyldiarylsilyl, triarylsilyl, aryl(C₁-C₄)alkylthio(C₁-C₄)alkyl, aryloxy(C₁-C₄)alkyl, formyl, C₁-C₁₀alkylcarbonyl, HO₂C, C₁-C₁₀alkoxycarbonyl, aminocarbonyl, C₁-C₆alkylaminocarbonyl, di(C₁-C₆ alkyl)aminocarbonyl, N—(C₁-C₃alkyl)-N—(C₁-C₃ alkoxy)aminocarbonyl, C₁-C₆alkylcarbonyloxy, arylcarbonyloxy (where the aryl group is optionally substituted), di(C₁-C₆)alkylaminocarbonyloxy, C₁-C₆alkyliminooxy, C₃-C₆alkenyloxyimino, aryloxyimino, aryl (itself optionally substituted), heteroaryl (itself optionally substituted), heterocyclyl (itself optionally substituted with C₁-C₆alkyl or halogen), aryloxy (where the aryl group is optionally substituted), heteroaryloxy, (where the heteroaryl group is optionally substituted), heterocyclyloxy (where the heterocyclyl group is optionally substituted with C₁-C₆alkyl or halogen), amino, C₁-C₆alkylamino, di(C₁-C₆)alkylamino, C₁-C₆alkylcarbonylamino, N—(C₁-C₆)alkylcarbonyl-N—(C₁-C₆)alkylamino, C₂-C₆alkenylcarbonyl, C₂-C₆alkynylcarbonyl, C₃-C₆alkenyloxycarbonyl, C₃-C₆alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that allenyl and alkylinylalkenyl are included in these terms.

When present, the optional substituents on alkenyl or alkynyl include those optional substituents given above for an alkyl moiety.

Halogen is fluorine, chlorine, bromine or iodine.

Haloalkyl groups are alkyl groups which are substituted with one or more of the same or different halogen atoms and are, for example, CF₃, CF₂C₁, CF₂H, CCl₂H, FCH₂, ClCH₂, BrCH₂, CH₃CHF, (CH₃)₂CF, CF₃CH₂ or CHF₂CH₂.

In the context of the present specification the term “aryl” refers to ring systems which may be mono-, bi- or tricyclic. Examples of such rings include phenyl, naphthyl, anthracenyl, indenyl or phenanthrenyl. A preferred aryl group is phenyl.

The term “heteroaryl” preferably refers to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl.

Preferred examples of heteroaromatic radicals include pyridyl, pyrimidinyl, triazinyl, thienyl, furyl, oxazolyl, isoxazolyl, 2,1,3-benzoxadiazolyl and thiazolyl.

Another group of preferred heteroaryls comprises pyrazolyl, 1,2,3-triazolyl, 1,2,4-triazolyl, pyridazinyl, pyrazinyl, quinolinyl, isoquinolinyl, cinnolinyl, quinazolinyl or quinoxalinyl.

The term “heterocyclyl” preferably refers to a non-aromatic preferably monocyclic or bicyclic ring systems containing up to 7 atoms including one or more (preferably one or two) heteroatoms selected from O, S and N. Examples of such rings include 1,3-dioxolane, oxetane, tetrahydrofuran, morpholine, thiomorpholin and piperazine. When present, the optional substituents on heterocyclyl include C₁-C₆alkyl and C₁-C₆haloalkyl as well as those optional substituents given above for an alkyl moiety.

Cycloalkyl includes preferably cyclopropyl, cyclobutyl, cyclopentyl and cyclohexyl. Cycloalkylalkyl is preferentially cyclopropylmethyl. Cycloalkenyl includes preferably cyclopentenyl and cyclohexenyl. When present, the optional substituents on cycloalkyl or cycloalkenyl include C₁-C₃alkyl as well as those optional substituents given above for an alkyl moiety.

Carbocyclic rings include aryl, cycloalkyl or carbocyclic groups, and cycloalkenyl groups.

When present, the optional substituents on aryl, heteroaryl and carbocycles are preferably selected independently, from halogen, nitro, cyano, rhodano, isothiocyanato, C₁-C₆alkyl, C₁-C₆haloalkyl, C₁-C₆alkoxy(C₁-C₆)alkyl, C₂-C₆alkenyl, C₂-C₆haloalkenyl, C₂-C₆alkynyl, C₃-C₇cycloalkyl (itself optionally substituted with C₁-C₆alkyl or halogen), C₅₋₇cycloalkenyl (itself optionally substituted with C₁-C₆alkyl or halogen), hydroxy, C₁-C₁₀alkoxy, C₁-C₁₀alkoxy(C₁-C₁₀)alkoxy, tri(C₁-C₄)alkylsilyl(C₁-C₆)alkoxy, C₁-C₆alkoxycarbonyl(C₁-C₁₀)alkoxy, C₁-C₁₀haloalkoxy, aryl(C₁-C₄)alkoxy (where the aryl group is optionally substituted with halogen or C₁-C₆alkyl), C₃-C₇cycloalkyloxy (where the cycloalkyl group is optionally substituted with C₁-C₆alkyl or halogen), C₃-C₁₀alkenyloxy, C₃-C₁₀alkynyloxy, mercapto, C₁-C₁₀alkylthio, C₁-C₁₀haloalkylthio, aryl(C₁-C₄)alkylthio, C₃-C₇cycloalkylthio (where the cycloalkyl group is optionally substituted with C₁-C₆alkyl or halogen), tri(C₁-C₄)-alkylsilyl(C₁-C₆)alkylthio, arylthio, C₁-C₆alkylsulfonyl, C₁-C₆haloalkylsulfonyl, C₁-C₆alkylsulfinyl, C₁-C₆haloalkylsulfinyl, arylsulfonyl, tri(C₁-C₄)alkylsilyl, aryldi(C₁-C₄)alkylsilyl, C₁-C₄alkyldiarylsilyl, triarylsilyl, C₁-C₁₀alkylcarbonyl, HO₂C, C₁-C₁₀alkoxycarbonyl, aminocarbonyl, C₁-C₆alkylaminocarbonyl, di(C₁-C₆alkyl)-aminocarbonyl, N—(C₁-C₃ alkyl)-N—(C₁-C₃alkoxy)aminocarbonyl, C₁-C₆alkylcarbonyloxy, arylcarbonyloxy, di(C₁-C₆)alkylaminocarbonyloxy, aryl (itself optionally substituted with C₁-C₆alkyl or halogen), heteroaryl (itself optionally substituted with C₁-C₆alkyl or halogen), heterocyclyl (itself optionally substituted with C₁-C₆alkyl or halogen), aryloxy (where the aryl group is optionally substituted with C₁-C₆alkyl or halogen), heteroaryloxy (where the heteroaryl group is optionally substituted with C₁-C₆alkyl or halogen), heterocyclyloxy (where the heterocyclyl group is optionally substituted with C₁-C₆alkyl or halogen), amino, C₁-C₆alkylamino, di(C₁-C₆)alkylamino, C₁-C₆alkylcarbonylamino, N—(C₁-C₆)alkylcarbonyl-N—(C₁-C₆)alkylamino, arylcarbonyl, (where the aryl group is itself optionally substituted with halogen or C₁-C₆alkyl) or two adjacent positions on an aryl or heteroaryl system may be cyclised to form a 5, 6 or 7 membered carbocyclic or heterocyclic ring, itself optionally substituted with halogen or C₁-C₆alkyl. Further substituents for aryl or heteroaryl include arylcarbonylamino (where the aryl group is substituted by C₁-C₆alkyl or halogen), (C₁-C₆)alkoxycarbonylamino, (C₁-C₆)alkoxycarbonyl-N—(C₁-C₆)alkylamino, aryloxycarbonylamino (where the aryl group is substituted by C₁-C₆alkyl or halogen), aryloxycarbonyl-N—(C₁-C₆)alkylamino, (where the aryl group is substituted by C₁-C₆alkyl or halogen), arylsulphonylamino (where the aryl group is substituted by C₁-C₆alkyl or halogen), arylsulphonyl-N—(C₁-C₆)alkylamino (where the aryl group is substituted by C₁-C₆alkyl or halogen), aryl-N—(C₁-C₆)alkylamino (where the aryl group is substituted by C₁-C₆alkyl or halogen), arylamino (where the aryl group is substituted by C₁-C₆alkyl or halogen), heteroarylamino (where the heteroaryl group is substituted by C₁-C₆alkyl or halogen), heterocyclylamino (where the heterocyclyl group is substituted by C₁-C₆alkyl or halogen), aminocarbonylamino, C₁-C₆alkylaminocarbonylamino, di(C₁-C₆)alkylaminocarbonylamino, arylaminocarbonylamino where the aryl group is substituted by C₁-C₆alkyl or halogen), aryl-N—(C₁-C₆)alkylaminocarbonylamino where the aryl group is substituted by C₁-C₆alkyl or halogen), C₁-C₆alkylaminocarbonyl-N—(C₁-C₆)alkylamino, di(C₁-C₆)alkylaminocarbonyl-N—(C₁-C₆)alkylamino, arylaminocarbonyl-N—(C₁-C₆)alkylamino where the aryl group is substituted by C₁-C₆alkyl or halogen) and aryl-N—(C₁-C₆)alkylaminocarbonyl-N—(C₁-C₆)alkylamino where the aryl group is substituted by C₁-C₆alkyl or halogen).

For substituted heterocyclyl groups it is preferred that one or more substituents are independently selected from halogen, C₁-C₆alkyl, C₁-C₆haloalkyl, C₁-C₆alkoxy, C₁-C₆haloalkoxy, C₁-C₆alkylthio, C₁-C₆alkylsulfinyl, C₁-C₆alkylsulfonyl, nitro and cyano. It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected C₁-C₆alkyl groups. When heterocyclic rings are formed by joining two groups on an N atom, the resulting rings are suitably pyrrolidine, piperidine, thiomorpholine and morpholine each of which may be substituted by one or two independently selected C₁-C₆alkyl groups.

The invention relates also to the agriculturally acceptable salts which the compounds of formula I are able to form with transition metal, alkali metal and alkaline earth metal bases, amines, quaternary ammonium bases or tertiary sulfonium bases.

Among the transition metal, alkali metal and alkaline earth metal salt formers, special mention should be made of the hydroxides of copper, iron, lithium, sodium, potassium, magnesium and calcium, and preferably the hydroxides, bicarbonates and carbonates of sodium and potassium.

Examples of amines suitable for ammonium salt formation include ammonia as well as primary, secondary and tertiary C₁-C₁₈alkylamines, C₁-C₄hydroxyalkylamines and C₂-C₄alkoxyalkyl-amines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, di-isopropylamine, di-n-butylamine, di-n-amylamine, di-isoamylamine, dihexyl-amine, diheptylamine, dioctylamine, ethanolamine, n-propanolamine, isopropanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, trimethylamine, triethylamine, tri-n-propylamine, tri-isopropylamine, tri-n-butylamine, tri-isobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and di-isopropylamine.

Preferred quaternary ammonium bases suitable for salt formation correspond, for example, to the formula [N(RₐR_{b}R_{c}R_{d})]OH, wherein Rₐ, R_{b}, R_{c} and R_{d} are each independently of the others hydrogen, C₁-C₄alkyl. Further suitable tetraalkylammonium bases with other anions can be obtained, for example, by anion exchange reactions.

Preferred tertiary sulfonium bases suitable for salt formation correspond, for example, to the formula [SRₑR_{f}R_{g}]OH, wherein Rₑ, R_{f} and R_{g} are each independently of the others C₁-C₄ alkyl. Trimethylsulfonium hydroxide is especially preferred. Suitable sulfonium bases may be obtained from the reaction of thioethers, in particular dialkylsulfides, with alkylhalides, followed by conversion to a suitable base, for example a hydroxide, by anion exchange reactions.

It should be understood that in those compounds of formula I, where G is a metal, ammonium or sulfonium as mentioned above and as such represents a cation, the corresponding negative charge is largely delocalised across the O—C═C—C═O unit.

The compounds of formula I according to the invention also include hydrates which may be formed during the salt formation.

The latentiating groups G are selected to allow its removal by one or a combination of biochemical, chemical or physical processes to afford compounds of formula I where G is H before, during or following application to the treated area or plants. Examples of these processes include enzymatic cleavage, chemical hydrolysis and photoloysis. Compounds bearing such groups G may offer certain advantages, such as improved penetration of the cuticula of the plants treated, increased tolerance of crops, improved compatibility or stability in formulated mixtures containing other herbicides, herbicide safeners, plant growth regulators, fungicides or insecticides, or reduced leaching in soils.

The latentiating group G is preferably selected from the groups C₁-C₈alkyl, C₂-C₈haloalkyl, phenylC₁-C₈alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano or by nitro), heteroarylC₁-C₈alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₈alkenyl, C₃-C₈haloalkenyl, C₃-C₈alkynyl, C(X^{a})—R^{a}, C(X^{b})—X^{c}—R^{b}, C(X^{d})—N(R^{c})—R^{d}, —SO₂—R^{e}, —P(X^{e})(R^{f})—R^{g} or CH₂—X^{f}—R^{h} wherein X^{a}, X^{b}, X^{c}, X^{d}, X^{e} and X^{f} are independently of each other oxygen or sulfur;

R^{a} is H, C₁-C₁₈alkyl, C₂-C₁₈alkenyl, C₂-C₁₈alkynyl, C₁-C₁₀haloalkyl, C₁-C₁₀cyanoalkyl, C₁-C₁₀nitroalkyl, C₁-C₁₀aminoalkyl, C₁-C₅alkylamino(C₁-C₈)alkyl, C₂-C₈dialkylamino(C₁-C₅)alkyl, C₃-C₇cycloalkyl(C₁-C₅)alkyl, C₁-C₅alkoxy(C₁-C₈)alkyl, C₃-C₅alkenyloxy(C₁-C₅)alkyl, C₃—(C₁-C₅)oxyalkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroaryl(C₁-C₅)alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro,

R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkyl-thio, C₁-C₃alkylsulfinyl, C₁-C₃alkylsulfonyl, halogen, cyano, or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro,

R^{c} and R^{d} are each independently of each other hydrogen, C₁-C₁₀alkyl, C₃-C₁₀alkenyl, C₃-C₁₀alkynyl, C₂-C₁₀haloalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroaryl(C₁-C₅)alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₈haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro, heteroarylamino or heteroarylamino substituted by C₁-C₃alkyl, C₁-C₃haloalkyl, C₁-C₃alkoxy, C₁-C₃haloalkoxy, halogen, cyano or nitro, diheteroarylamino or diheteroarylamino substituted by C₁-C₃alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or by nitro, diphenylamino or diphenylamino substituted by C₁-C₃alkyl, C₁-C₃haloalkyl, C₁-C₃alkoxy, C₁-C₃haloalkoxy, halogen, cyano or by nitro or C₃-C₇cycloalkylamino, di-C₃-C₇cycloalkylamino or C₃-C₇cycloalkoxy or R^{c} and R^{d} may join together to form a 3-7 membered ring, optionally containing one heteroatom selected from O or S,

R^{e} is C₁-C₁₀alkyl, C₂-C₁₀alkenyl, C₂-C₁₀alkynyl, C₁-C₁₀haloalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₈trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroaryl(C₁-C₅)alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀alkoxy, C₁-C₁₀haloalkoxy, C₁-C₅alkylamino or C₂-C₈dialkylamino,

R^{f} and R^{g} are each independently of each other C₁-C₁₀alkyl, C₂-C₁₀alkenyl, C₂-C₁₀alkynyl, C₁-C₁₀alkoxy, C₁-C₁₀haloalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroaryl(C₁-C₅)alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀haloalkoxy, C₁-C₅alkylamino or C₂-C₈dialkylamino, benzyloxy or phenoxy, wherein the benzyl and phenyl groups may in turn be substituted by C₁-C₃alkyl, C₁-C₃haloalkyl, C₁-C₃alkoxy, C₁-C₃haloalkoxy, halogen, cyano or nitro, and

R^{h} is C₁-C₁₀alkyl, C₃-C₁₀alkenyl, C₃-C₁₀alkynyl, C₁-C₁₀haloalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroaryl(C₁-C₅)alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), phenoxy(C₁-C₅)alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroaryloxy(C₁-C₅)alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen or by nitro, or heteroaryl, or heteroaryl substituted by C₁-C₃alkyl, C₁-C₃haloalkyl, C₁-C₃alkoxy, C₁-C₃haloalkoxy, halogen, cyano or by nitro.

In particular, the latentiating group G is a group —C(X^{a})—R^{a} or —C(X^{b})—X^{c}—R^{b}, and the meanings of X^{a}, R^{a}, X^{b}, X^{c} and R^{b} are as defined above.

It is preferred that G is hydrogen, an alkali metal or alkaline earth metal, where hydrogen is especially preferred.

Depending on the nature of the substituents, compounds of formula I may exist in different isomeric forms. When G is hydrogen, for example, compounds of formula I may exist in different tautomeric forms:

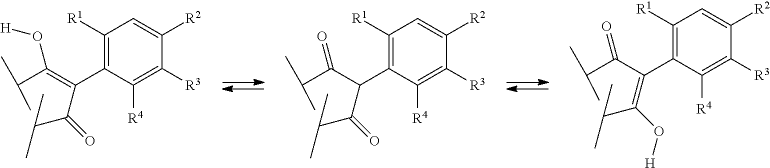

This invention covers all such isomers and tautomers and mixtures thereof in all proportions. Also, when substituents contain double bonds, cis- and trans-isomers can exist. These isomers, too, are within the scope of the claimed compounds of the formula I.

Preferably, in the compounds of the formula (I), R¹ is methyl, ethyl, n-propyl, cyclopropyl, halomethyl, haloethyl, halogen, vinyl or ethynyl. More preferably, R¹ is methyl, ethyl, cyclopropyl or chlorine, where methyl and ethyl are particularly preferred.

Preferably, R² and R³ are independently of each other hydrogen, phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃ haloalkoxy, cyano, nitro or halogen.

Preferably, R⁴ is hydrogen, methyl or ethyl.

In a preferred group of compounds of the formula (I), R¹ is ethyl, R² is hydrogen, R³ is phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃haloalkoxy, cyano, nitro or halogen, and R⁴ is hydrogen.

In another preferred group of compounds of the formula (I), R¹ is methyl or ethyl, R² is phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃haloalkoxy, cyano, nitro or halogen, R³ is hydrogen and R⁴ is hydrogen, methyl or ethyl.

In another preferred group of compounds of the formula (I), R¹ is methyl or ethyl, R² is methyl, R³ is hydrogen and R⁴ is methyl or ethyl.

Most preferably, R¹ is ethyl, R² is hydrogen, R³ is phenyl substituted in the para-position by chlorine, bromine or iodine, especially chlorine, and optionally further substituted once or twice by C₁-C₄alkyl, C₁-C₃haloalkyl, C₁-C₃alkoxy, C₁-C₃haloalkoxy, cyano, nitro or halogen, and R⁴ is hydrogen.

Preferably, R⁵ and R⁸ are independently of each other hydrogen or methyl. More preferably, R⁵ and R⁸ are hydrogen.

Preferably, R⁶ and R⁷ are independently of each other hydrogen, optionally substituted C₁-C₆alkyl, optionally substituted C₂-C₆alkenyl, optionally substituted C₁-C₆alkoxy or tri-C₁-C₄alkylsilyloxy. More preferably, R⁶ and R⁷ are independently of each other hydrogen, optionally substituted C₁-C₆alkyl or optionally substituted C₁-C₆alkoxy, where hydrogen is particularly preferred.

Preferably, X is optionally substituted methylene or ethylene. More preferably, X is methylene or ethylene, or methylene or ethylene substituted once or twice by methyl, where methylene or ethylene, and especially ethylene is particularly preferred.

Preferably, W is —CR⁹═CR¹⁰— or —CHR⁹—CHR¹⁰— wherein R⁹ and R¹⁰ are independently of each other hydrogen, optionally substituted C₁-C₆alkyl, optionally substituted C₂-C₆alkenyl, optionally substituted C₁-C₆alkoxy or tri-C₁-C₄alkylsilyloxy, or is a fragment —CH₂—C(O)— or —CH₂—C(═NOR¹¹)—, wherein R¹¹ is C₁-C₆alkyl. More preferably, W is —CR⁹═CR¹⁰— or —CHR⁹—CHR¹⁰— wherein R⁹ and R¹⁰ are independently of each other hydrogen, optionally substituted C₁-C₆alkyl, optionally substituted C₂-C₆alkenyl, optionally substituted C₁-C₆alkoxy or tri-C₁-C₄alkylsilyloxy, where —CR⁹═CR¹⁰— or —CHR⁹—CHR¹⁰—, wherein R⁹ and R¹⁰ are hydrogen, is particularly preferred.

A compound of formula (I) wherein G is C₁-C₈alkyl, C₂-C₈haloalkyl, phenylC₁-C₈alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsufinyl, C₁-C₃alkylsulfonyl, halogen, cyano or by nitro), heteroarylC₁-C₈alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsufinyl, C₁-C₃alkylsulfonyl, halogen, cyano or by nitro), C₃-C₈alkenyl, C₃-C₈haloalkenyl, C₃-C₆alkynyl, C(X^{a})—R^{a}, C(X^{b})—X^{c}—R^{b}, C(X^{d})—N(R^{c})—R^{d}, —SO₂—R^{e}, —P(X^{e})(R^{f})—R^{g} or CH₂—X^{f}—R^{h} where X^{a}, X^{b}, X^{c}, X^{d}, X^{e}, X^{f}, R^{a}, R^{b}, R^{c}, R^{d}, R^{e}, R^{f}, R^{g} and R^{h} are as defined above may be prepared by treating a compound of formula (A), which is a compound of formula (I) wherein G is H, with a reagent G-Z, wherein G-Z is an alkylating agent such as an alkyl halide (the definition of alkyl halides includes simple C₁-C₈ alkyl halides such as methyl iodide and ethyl iodide, substituted alkyl halides such as chloromethyl alkyl ethers, C₁—CH₂—X^{f}—R^{h}, wherein X^{f} is oxygen, and chloromethyl alkyl sulfides C₁—CH₂—X^{f}—R^{h}, wherein X^{f} is sulfur), a C₁-C₈alkyl sulfonate, or a di(C₁-C₈alkyl)sulfate, or with a C₃-C₈alkenyl halide, or with a C₃-C₈alkynyl halide, or with an acylating agent such as a carboxylic acid, HO—C(X^{a})R^{a}, wherein X^{a} is oxygen, an acid chloride, Cl—C(X^{a})R^{a}, wherein X^{a} is oxygen, or acid anhydride, [R^{a}C(X^{a})]₂O, wherein X^{a} is oxygen, or an isocyanate, R^{c}N═C═O, or a carbamoyl chloride, Cl—C(X^{d})—N(R^{c})—R^{d} (wherein X^{d} is oxygen and with the proviso that neither R^{c} or R^{d} is hydrogen), or a thiocarbamoyl chloride Cl—C(X^{d})—N(R^{c})—R^{d} (wherein X^{d} is sulfur and with the proviso that neither R^{c} or R^{d} is hydrogen) or a chloroformate, Cl—C(X^{b})—X^{c}—R^{b}, (wherein X^{b} and X^{c} are oxygen), or a chlorothioformate Cl—C(X^{b})—X^{c}—R^{b} (wherein X^{b} is oxygen and X^{c} is sulfur), or a chlorodithioformate Cl—C(X^{b})—X^{c}—R^{b}, (wherein X^{b} and X^{c} are sulfur), or an isothiocyanate, R^{c}N═C═S, or by sequential treatment with carbon disulfide and an alkylating agent, or with a phosphorylating agent such as a phosphoryl chloride, Cl—P(X^{e})(R^{f})—R^{g} or with a sulfonylating agent such as a sulfonyl chloride Cl—SO₂—R^{e}, preferably in the presence of at least one equivalent of base. Where a compound of formula (A) is asymmetric, for example when substituent R⁵ is not equal to substituent R⁸, or substituent R⁶ is not equal to substituent R⁷, these reactions may produce, in addition to a compound of formula (I), a second compound of formula (IA). This invention covers both a compound of formula (I) and a compound of formula (IA), together with mixtures of these compounds in any ratio.

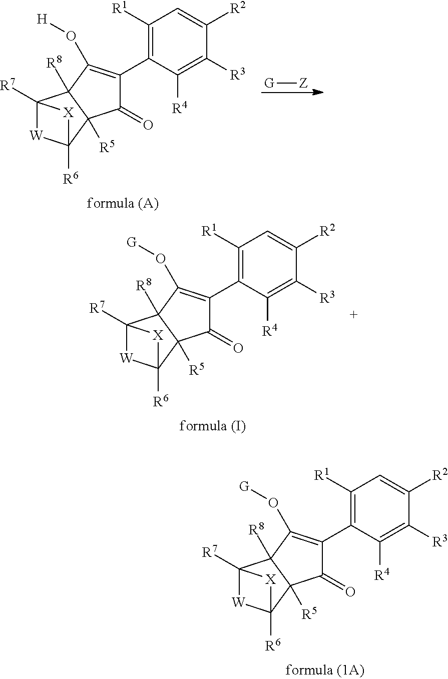

The O-alkylation of cyclic 1,3-diones is known; suitable methods are described, for example, by T. Wheeler, U.S. Pat. No. 4,436,666. Alternative procedures have been reported by M. Pizzorno and S. Albonico, Chem. Ind. (London), (1972), 425-426; H. Born et al., J. Chem. Soc., (1953), 1779-1782; M. Constantino et al., Synth. Commun., (1992), 22 (19), 2859-2864; Y. Tian et al., Synth. Commun., (1997), 27 (9), 1577-1582; S. Chandra Roy et al., Chem. Letters, (2006), 35 (1), 16-17; P. K. Zubaidha et al., Tetrahedron Lett., (2004), 45, 7187-7188.

The O-acylation of cyclic 1,3-diones may be effected by procedures similar to those described, for example, by R. Haines, U.S. Pat. No. 4,175,135, and by T. Wheeler, U.S. Pat. No. 4,422,870, U.S. Pat. No. 4,659,372 and U.S. Pat. No. 4,436,666. Typically diones of formula (A) may be treated with an acylating agent preferably in the presence of at least one equivalent of a suitable base, and optionally in the presence of a suitable solvent. The base may be inorganic, such as an alkali metal carbonate or hydroxide, or a metal hydride, or an organic base such as a tertiary amine or metal alkoxide. Examples of suitable inorganic bases include sodium carbonate, sodium or potassium hydroxide, sodium hydride, and suitable organic bases include trialkylamines, such as trimethylamine and triethylamine, pyridines or other amine bases such as 1,4-diazobicyclo[2.2.2]-octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent. For cases where the acylating agent is a carboxylic acid, acylation is preferably effected in the presence of a known coupling agent such as 2-chloro-1-methylpyridinium iodide, N,N′-dicyclohexylcarbodiimide, 1-(3-dimethylaminopropyl)-3-ethylcarbodiimide and N,N′-carbodiimidazole, and optionally in the presence of a base such as triethylamine or pyridine in a suitable solvent such as tetrahydrofuran, dichloromethane or acetonitrile. Suitable procedures are described, for example, by W. Zhang and G. Pugh, Tetrahedron Lett., (1999), 40 (43), 7595-7598; T. Isobe and T. Ishikawa, J. Org. Chem., (1999), 64 (19), 6984-6988 and K. Nicolaou et al., (2005), 127(24), 8872-8888.

Phosphorylation of cyclic 1,3-diones may be effected using a phosphoryl halide or thiophosphoryl halide and a base by procedures analogous to those described by L. Hodakowski, U.S. Pat. No. 4,409,153.

Sulfonylation of a compound of formula (A) may be achieved using an alkyl or aryl sulfonyl halide, preferably in the presence of at least one equivalent of base, for example by the procedure of C. Kowalski and K. Fields, J. Org. Chem., (1981), 46, 197-201.

A compound of formula (A), wherein W is an optionally substituted ethylene group, —CHR⁹—CHR¹⁰—, may be prepared from an alkene of formula (B) by reduction, for example by catalytic hydrogenation, or by reaction with diimide, in a suitable solvent. The reduction is preferably carried out by hydrogenation in the presence of a suitable metal catalyst (such as a palladium or platinum catalyst), and in a suitable solvent such as methanol, ethanol or ethyl acetate.

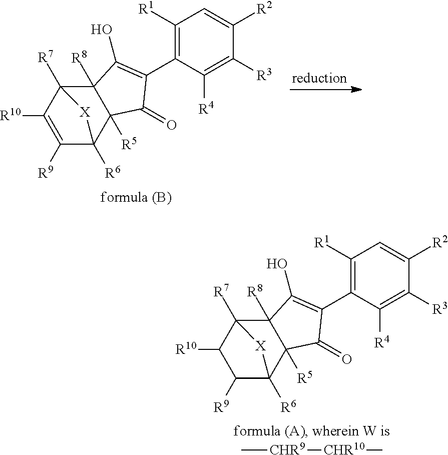

Compounds of formula (B) are alkenes and as such undergo further reactions typical of alkenes to give additional compounds of formula (A) according to known procedures. Examples of such reactions include, but are not restricted to, halogenation, epoxidation, cyclopropanation, dihydroxylation, hydroarylation, hydrovinylation and hydration of alkenes. Compounds of formula (B) wherein R⁹ or R¹⁰ are bromine or iodine are vinyl halides, and undergo known reactions of vinyl halides such as Suzuki-Miyaura, Sonogashira, Stille and related reactions. Compounds of formula (B) wherein R⁹ or R¹⁰ are C₁-C₆alkoxy are enol ethers, and these may be hydrolysed to the corresponding ketone using known procedures to give additional compounds of formula (A).

In turn, these products may be transformed into additional compounds of formula (A), such as oximes, imines, hydrazones and the like, by methods described, for example by J. March, Advanced Organic Chemistry, third edition, John Wiley and Sons.

A compound of formula (B) may be prepared from a compound of formula (C) by reaction with a diene of formula (D), optionally in the presence of a suitable solvent, and a suitable catalyst, and optionally under microwave irradiation.

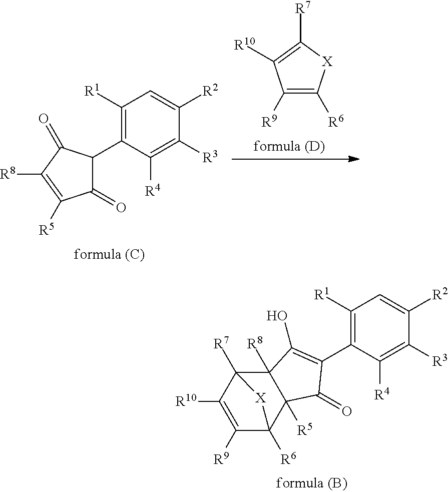

Suitable solvents include toluene, dichloromethane, chloroform, acetone, 4-methylpentan-2-one, methanol, ethanol, water and ionic liquids, as described, for example by G. Silvero et al., Tetrahedron (2005), 61, 7105-7111; I. Hemeon et al., Synlett, (2002), 11, 1815-1818; S. Otto and J. Engberts, Pure Appl. Chem. (2000), 72 (7), 1365-1372; and by R. Breslow, Acc. Chem. Res., (1991), 24 (6), 159-164. Suitable catalysts include Lewis acid catalysts (such as those described, for example by K. Hara et al., Org. Lett., (2005), 7 (25), 5621-5623; J, Auge et al., Synlett, (2000), 6, 877-879, B. Garrigues and A. Oussaid, J. Organometallic Chem., (1989), 585, 253-255; B. Mathieu and L. Ghosez, Tetrahedron Lett., (1997), 38 (31), 5497-5500; M. Ordoñez et al., Tetrahedron Asymmetry, (1996), 7 (9), 2675-2686; S. Kobayashi et al., Tetrahedron Lett., (1993), 34 (23), 3755-3758; C. Cativiela et al., U. Pindur et al., Chem. Rev., (1993), 93, 741-761; Tetrahedron, (1992), 48 (31), 6467-6476; J. Aube et al., J. Am. Chem. Soc., (1992), 114, 5466-5467; S. Danishefsky and M. Bednarski, Tetrahedron Lett., (1985), 26 (21), 2507-2508 and references therein) and organocatalysts such as those described by, for example, Q. Chu, W. Zhang and D. Curran, Tetrahedron Lett., (2006), 47, 9287-9290; K. Ishihara and K. Nakano, J. Am. Chem. Soc., (2005), 127 (30), 10504-10505; and A. Northrup and D. MacMillan, (2002), J. Am. Chem. Soc., 124 (11), 2458-2460. Preferably the catalyst is a Lewis acid catalyst such as aluminium chloride, bismuth (III) chloride, bismuth (III) trifluoromethanesulfonate, boron trifluoride, cerium (III) chloride, copper (I) trifluoromethanesulfonate, diethylaluminium chloride, hafnium (IV) chloride, iron (III) chloride, lithium perchlorate, lithium trifluoromethanesulfonate, magnesium bromide, magnesium iodide, scandium (III) trifluoromethanesulfonate, tin (IV) chloride, titanium (IV) chloride, titanium (IV) isopropoxide, trimethyl aluminium, N-trimethylsilyl-bis(trifluoromethanesulfonyl)imide, trimethylsilyl trifluoromethane-sulfonate, ytterbium (III) trifluoromethanesulfonate, zinc iodide and zirconium (IV) chloride. Magnesium bromide and magnesium iodide are particularly preferred.

Compounds of formula (D) are known compounds, or may be made by known methods from known compounds.

A compound of formula (C) may be prepared from a compound of formula (E), wherein Hal is bromine or iodine, and a compound of formula (F) by methods similar to those described by K. Saito and H. Yamachika. U.S. Pat. No. 4,371,711.

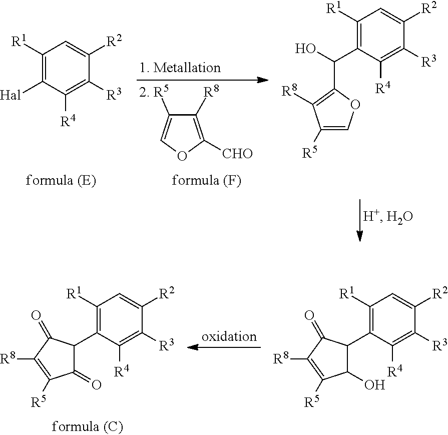

An aryl halide of formula (E) may be prepared from an aniline of formula (G) by known methods, for example the Sandmeyer reaction, via a suitable diazonium salt.

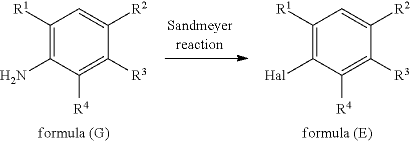

An aniline of formula (G) may be made by the cross-coupling of an aryl halide of formula (H), wherein Hal is chlorine, bromine or iodine, with a suitable coupling partners such as an aryl- or heteroarylboronic acid, R³—B(OH)₂, a suitable aryl- or heteroarylboronate ester, R³—B(OR)₂, (preferably an ester wherein the fragment —B(OR)₂ represents a cyclic boronate ester derived from a 1,2- or a 1,3-alkanediol, such as pinacol, 2,2-dimethyl-1,3-propanediol and 2-methyl-2,4-pentanediol), or a metal (especially potassium)aryl-, or heteroaryltrifluoroborate salt, M⁺[R³—BF₃]⁻ in the presence of a suitable palladium catalyst, a suitable ligand and a suitable base in the presence of a suitable solvent, under Suzuki-Miyaura conditions.

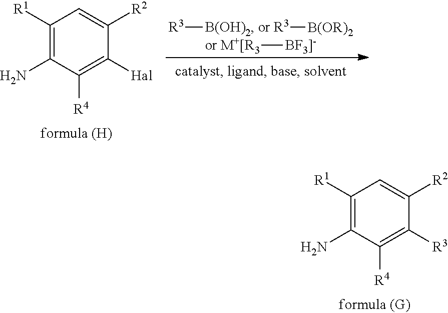

Conditions for effecting the Suzuki-Miyaura cross-coupling of a compound of formula (H) with an aryl- or heteroarylboronic acid of formula R³—B(OH)₂, or a suitable salt or ester thereof, are known in the literature (see, for example K. Billingsley and S. Buchwald, J. Am. Chem. Soc., (2007), 129, 3358-3366; H. Stefani, R. Cella and A. Vieira, Tetrahedron, (2007), 63, 3623-3658; N. Kudo, M. Perseghini and G. Fu, Angew. Chem. Int. Ed., (2006), 45, 1282-1284; A. Roglans, A. Pla-Quintana and M. Moreno-Mañas, Chem. Rev., (2006), 106, 4622-4643; J-H Li, Q-M Zhu and Y-X Xie, Tetrahedron (2006), 10888-10895; S, Nolan et al., J. Org. Chem., (2006), 71, 685-692; M. Lysën and K. Köhler, Synthesis, (2006), 4, 692-698; K. Anderson and S. Buchwald, Angew. Chem. Int. Ed., (2005), 44, 6173-6177; Y. Wang and D. Sauer, Org. Lett., (2004), 6 (16), 2793-2796; I. Kondolff, H. Doucet and M, Santelli, Tetrahedron, (2004), 60, 3813-3818; F. Bellina, A. Carpita and R. Rossi, Synthesis (2004), 15, 2419-2440; H. Stefani, G. Molander, C-S Yun, M. Ribagorda and B. Biolatto, J. Org. Chem., (2003), 68, 5534-5539; A. Suzuki, Journal of Organometallic Chemistry, (2002), 653, 83; G. Molander and C-S Yun, Tetrahedron, (2002), 58, 1465-1470; G. Zou, Y. K. Reddy and J. Falck, Tetrahedron Lett., (2001), 42, 4213-7215; S. Darses, G. Michaud and J-P. Genêt, Eur. J. Org. Chem., (1999), 1877-1883; M. Beavers et al., WO2005/012243; J. Org. Chem. (1994), 59, 6095-6097; A. Collier and G. Wagner, Synthetic Communications, (2006), 36; 3713-3721).

Compounds of formula (H) are known compounds, or may be made by known methods from known compounds. For example, a compound of formula (H) may be prepared from a nitrobenzene of formula (J) by reduction by known methods (for example by treatment with a reducing agent such as iron or zinc in the presence of an acid, or by catalytic hydrogenation).

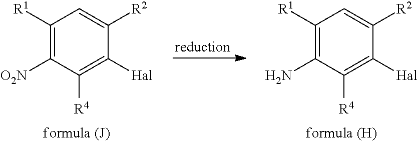

In an alternative approach to a compound of formula (G), a compound of formula (J) may be cross-coupled with a suitable aryl- or heteroaryl boronic acid, R³—B(OH)₂, or a suitable ester, R³—B(OR)₂, or salt, M⁺[R₃—BF₃]⁻, thereof, under Suzuki-Miyaura conditions, and the resulting nitrobenzene of formula (K) may be reduced under known conditions (for example by treatment with a reducing agent such as iron or zinc in the presence of an acid, or by catalytic hydrogenation) to give a compound of formula (G).

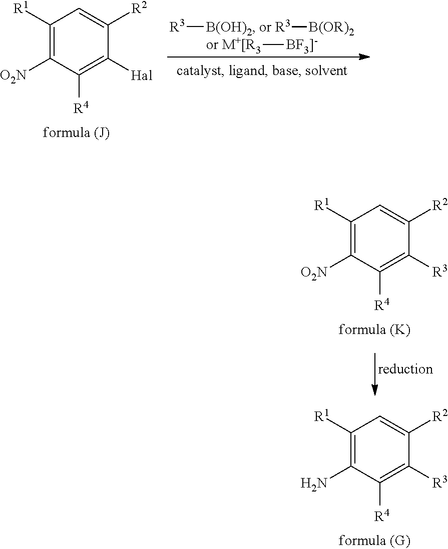

Compounds of formula (J) are known compounds, or may be made from known compounds by known methods. For example, a compound of formula (J) may be prepared by the Sandmeyer reaction of the corresponding aniline of formula (L), itself prepared from an aniline of formula (M) by nitration under acidic conditions.

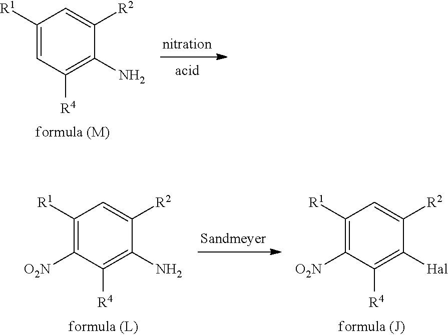

By similar methods, a compound of formula (G) may also be prepared from a compound of formula (N) via a compound of formula (K), or via a compound of formula (O).

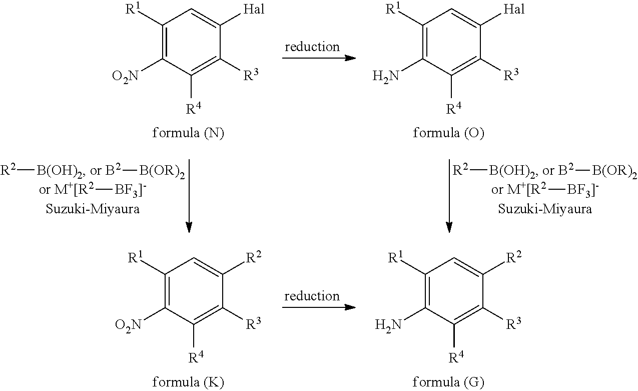

A compound of formula (N) may be prepared by nitration of a compound of formula (O), or by oxidation of aniline of formula (P) under known conditions. An aniline of formula (P) may be prepared by reduction of a compound of formula (N), or by halogenating an aniline of formula (Q), or by halogenating an anilide such as an acetanilide of formula (R) and hydrolysing the resulting amide under known conditions.

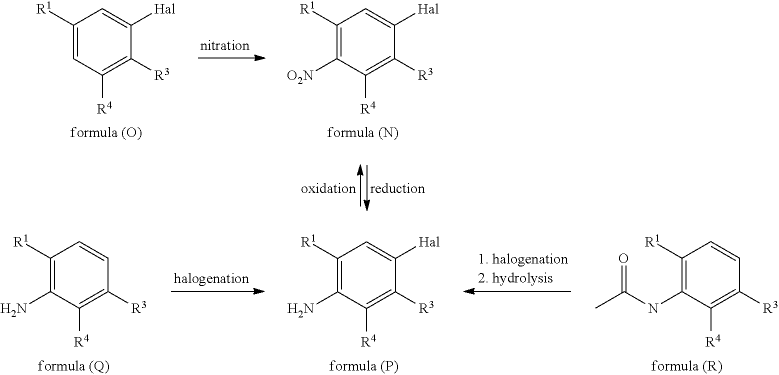

In an alternative approach, a compound of formula (A) may be prepared by cross-coupling an aryl halide of formula (S), wherein Hal is chlorine, bromine or iodine, with a suitable coupling partner such as an aryl- or heteroarylboronic acid, R³—B(OH)₂, an aryl- or heteroarylboronate ester, R³—B(OR)₂, wherein R is as defined previously, or a metal (especially potassium) aryl-, or heteroaryltrifluoroborate salt, M⁺[R³—BF₃]⁻ in the presence of a suitable palladium catalyst, a suitable ligand and a suitable base in the presence of a suitable solvent, under Suzuki-Miyaura conditions.

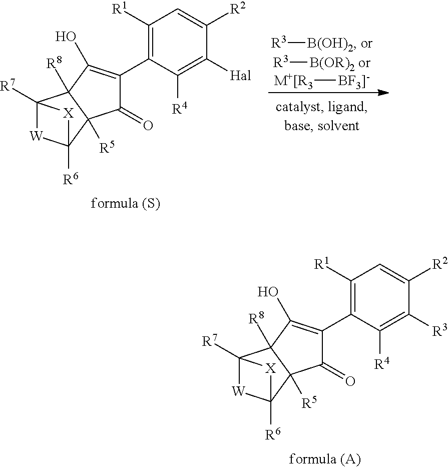

Alternatively, a compound of formula (S) may be converted into a compound of formula (A), by first converting it into an arylboronic acid, of formula (T), or a suitable ester or salt thereof, followed by cross-coupling with an aryl- or heteroaryl halide, R³-Hal (wherein Hal is chlorine, bromine or iodine) under Suzuki-Miyaura conditions. The conversion of a compound of formula (S) to a compound of formula (T) may be effected by treatment with at least two equivalents of a suitable metallating agent such as an alkyl lithium or an alkyl magnesium halide in a solvent such as tetrahydrofuran or diethyl ether, or by treatment with at least one equivalent of a suitable base (such as sodium hydride) followed by treatment of the resulting anion with at least one equivalent of a suitable metallating agent in a suitable solvent such as tetrahydrofuran or diethyl ether, and reacting the resulting organometallic species with a suitable borylating agent such as trimethylborate, to give an arylboronate of formula (U). An aryl boronate of formula (U) may be hydrolysed under acidic conditions to give an arylboronic acid of formula (T) for coupling under Suzuki-Miyaura conditions to give a compound of formula (A). Alternatively a compound of formula (S) may be reacted with a borylating reagent, H—B(OR)₂, or (RO)₂B—B(OR)₂, wherein R is as defined previously, under known conditions (see, for example, M. Miruta et al., Synlett, (2006), 12, 1867-1870; N. Miyaura et al., J. Org. Chem., (1995), 60, 7508, and W. Zhu and D. Ma, Org. Lett., (2006), 8 (2), 261), to give a compound of formula (V). Suitable borylating reagents include bis(pinacolato)diboron, bis(neopentyl glycolato)diboron, bis(hexylene glcolato)diboron and 4,4,5,5-tetramethyl-1,3,2-dioxaborolane. An arylboronate of formula (V) may be coupled under known Suzuki-Miyaura conditions to give a compound of formula (A).

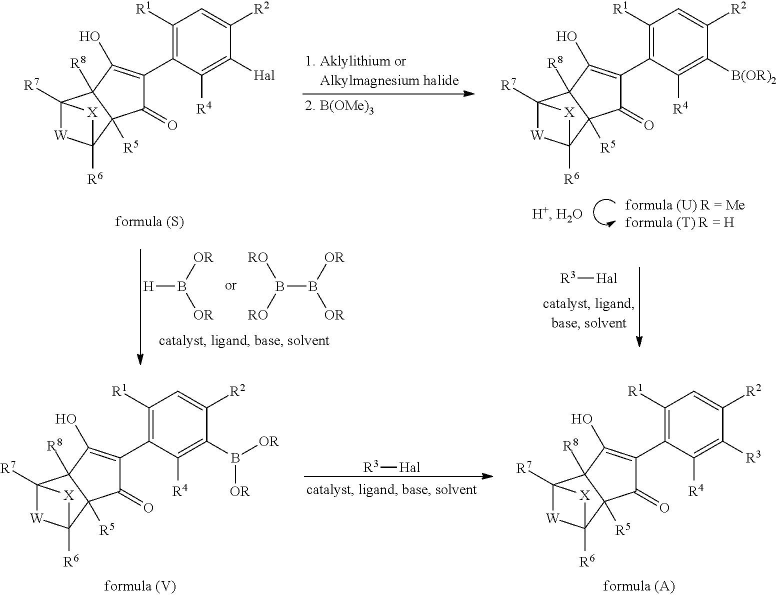

Using similar procedures, a compound of formula (A) may be prepared from a compound of formula (W) via a boronic acid of formula (Y) or via a boronate of formula (Z).

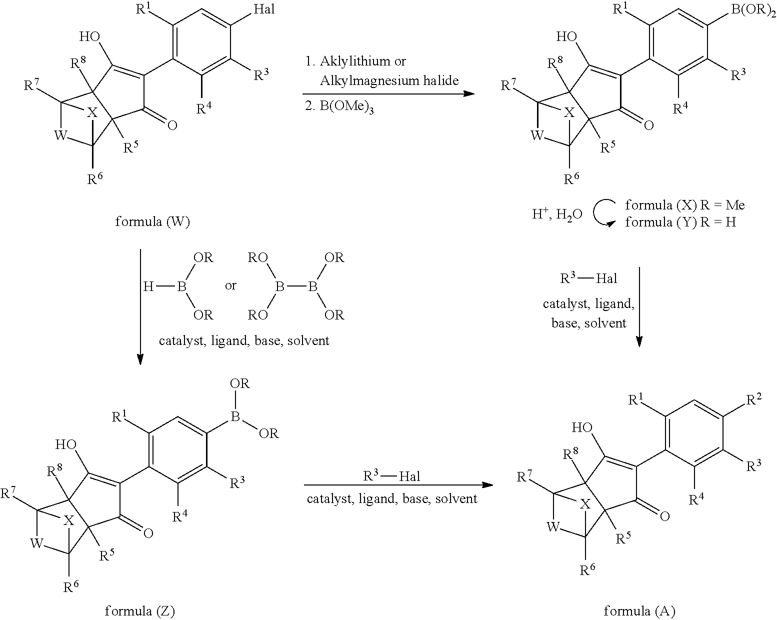

A compound of formula (S), wherein W is an optionally substituted ethylene group, —CR⁹—CR¹⁰—, and Hal is bromine or chlorine, may be prepared from a compound of formula (AA) by procedures analogous to those described for the preparation of a compound of formula (A), from a compound of formula (AA).

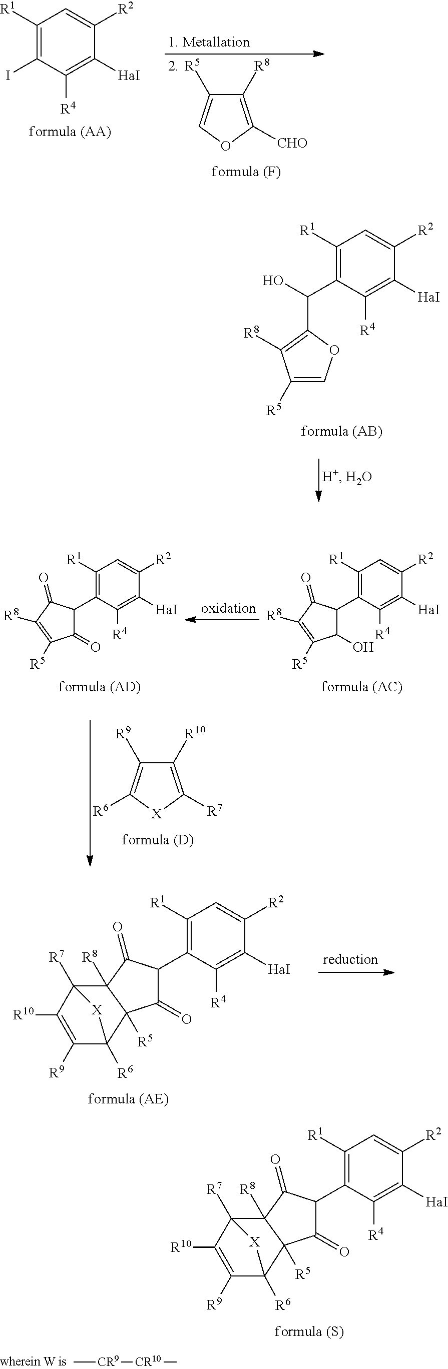

In a similar way, a compound of formula (W), wherein W is an optionally substituted ethylene group, —CR⁹—CR¹⁰—, and Hal is bromine or chlorine, may be prepared from a compound of formula (AF).

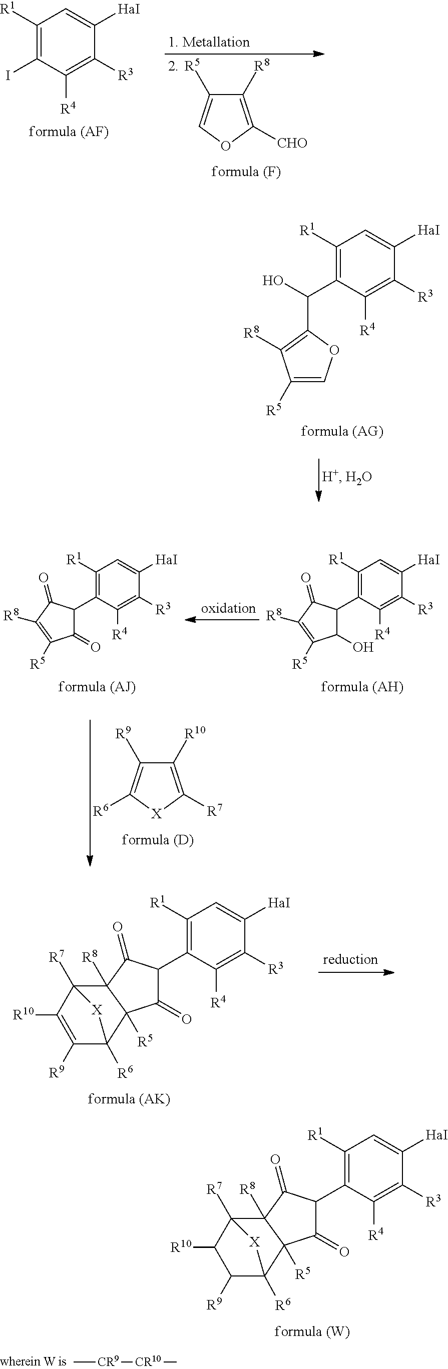

Compounds of formula (AA) and of formula (AF) are known compounds (see, for example, K. Okano et al., J. Am Chem. Soc., (2006), 128 (48), 15368-15369; M. Gubler et al., WO 2007/137962; E. Priestley et al., WO 2007/076431; M. Lautens et al., J. Org. Chem., (2001), 66, 8127-8134) or may be made by known methods from known compounds. For example, an aniline of formula (H) may be converted to a compound of formula (AA) under Sandmeyer conditions.

Additional compounds of formula (A) may be prepared by reacting an iodonium ylide of formula (AL), wherein Ar is an optionally substituted phenyl group, with an aryl boronic acid of formula (AM), in the presence of a suitable palladium catalyst and a base and in a suitable solvent.

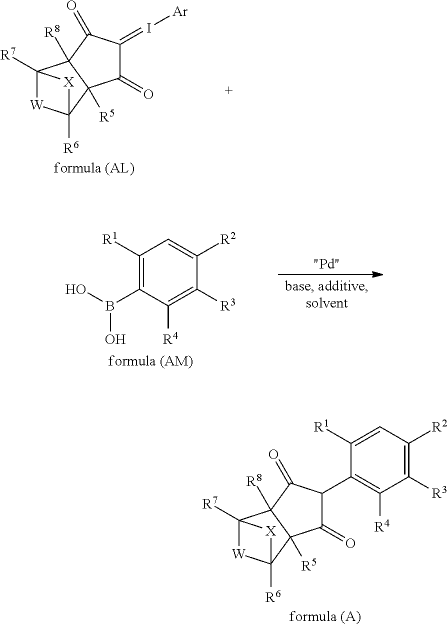

Suitable palladium catalysts are generally palladium(II) or palladium(0) complexes, for example palladium(II) dihalides, palladium(II) acetate, palladium(II) sulfate, bis(triphenylphosphine)palladium(II) dichloride, bis(tricyclopentylphosphine)palladium(II) dichloride, bis(tricyclohexylphosphine)palladium(II) dichloride, bis(dibenzylideneacetone)palladium(0) or tetrakis(triphenylphosphine)palladium(0). The palladium catalyst can also be prepared “in situ” from palladium(II) or palladium(0) compounds by complexing with the desired ligands, by, for example, combining the palladium(II) salt to be complexed, for example palladium(II) dichloride (PdCl₂) or palladium(II) acetate (Pd(OAc)₂), together with the desired ligand, for example triphenylphosphine (PPh₃), tricyclopentylphosphine, tricyclohexylphosphine, 2-dicyclohexyl-phosphino-2′,6′-dimethoxybiphenyl or 2-dicyclohexylphosphino-2′,4′,6′-triisopropylbiphenyl and the selected solvent, with a compound of formula (N), the arylboronic acid of formula (O), and a base. Also suitable are bidendate ligands, for example 1, 1′-bis(diphenylphosphino)ferrocene or 1,2-bis(diphenylphosphino)-ethane. By heating the reaction medium, the palladium(II) complex or palladium(0) complex desired for the C—C coupling reaction is thus formed “in situ”, and then initiates the C—C coupling reaction.

The palladium catalysts are used in an amount of from 0.001 to 50 mol %, preferably in an amount of from 0.1 to 15 mol %, based on the compound of formula (N). The reaction may also be carried out in the presence of other additives, such as tetralkylammonium salts, for example, tetrabutylammonium bromide. Preferably the palladium catalyst is palladium acetate, the base is lithium hydroxide and the solvent is aqueous 1,2-dimethoxyethane.

A compound of formula (AL) may be prepared from a compound of formula (AN) by treatment with a hypervalent iodine reagent such as a (diacetoxy)iodobenzene or an iodosylbenzene and a base such as aqueous sodium carbonate, lithium hydroxide or sodium hydroxide in a solvent such as water or an aqueous alcohol such as aqueous ethanol according to the procedures of K. Schank and C. Lick, Synthesis, (1983), 392; R. Moriarty et al, J. Am. Chem. Soc, (1985), 107, 1375, or of Z. Yang et al., Org. Lett., (2002), 4 (19), 3333.

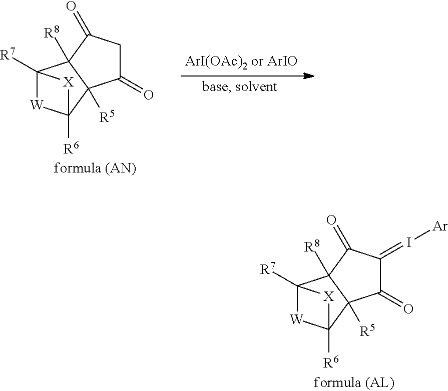

Compounds of formula (AN) are known compounds, or may be made by known methods from known compounds. For example, a compound of formula (AO), which is a compound of formula (AN) wherein W is an optionally substituted ethylene group, —CR⁹—CR¹⁰—, may be prepared from a compound of formula (AP) by reduction under known conditions (for example by catalytic hydrogenation).

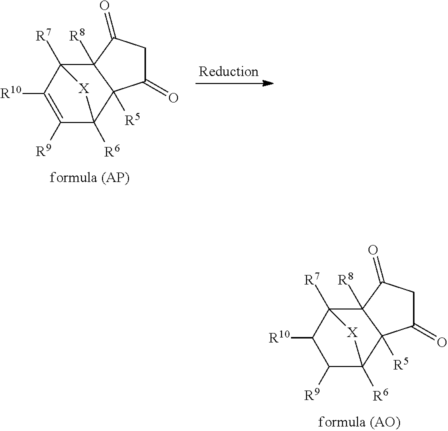

A compound of formula (AP) may be prepared by a Diels-Alder reaction between a compound of formula (D) and a cyclopentenedione of formula (AQ) under known conditions (see, for example, N. Ramesh et al., Tetrahedron (2001); 57, 9877-9887; F. Dutton, WO 93/14062; L. Paquette et al., J. Am. Chem. Soc., (1989), 111, 5792-5800; D. Buckle et al., J. Med. Chem., (1975), 18 (2), 203-206); C. DePuy and E. Zaweski, J. Am. Chem. Soc., (1959), 81, 4920-4924)

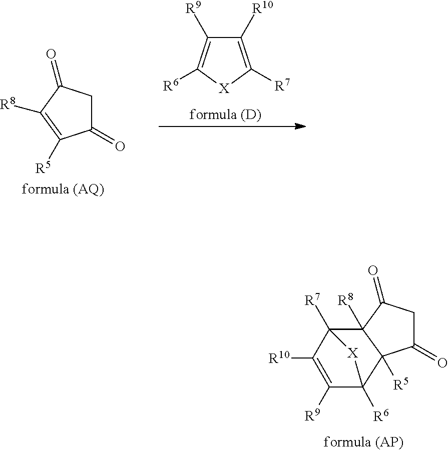

Compounds of formula (AQ) are known compounds, or may be made from known compounds by known methods.

A compound of formula (A) may also be prepared from a compound of formula (AN) by treatment with an aryllead tricarboxylate of formula (AR), wherein R′ is C₁-C₄ alkyl, in the presence of a suitable ligand in a suitable solvent.

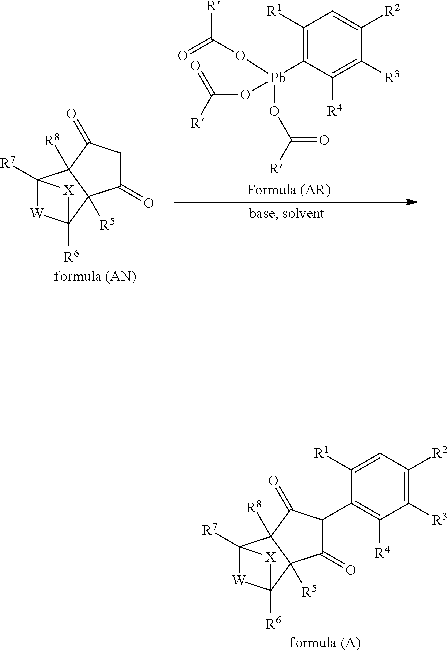

Preferably the aryllead triacetate of formula (AR) is an aryllead triacetate, and the reaction is effected in the presence of a suitable ligand (for example N,N-dimethylaminopyridine, pyridine, imidazole, bipyridine, and 1,10-phenanthroline, most preferably one to ten equivalents of N,N-dimethylaminopyridine with respect to compound of formula (AN)) and in a suitable solvent (for example chloroform, dichloromethane and toluene, preferably chloroform and optionally in the presence of a co-solvent such as toluene) at 25° C. to 100° C. (preferably 60-90° C.). Similar reactions are described in the literature (for example see, J. Pinhey, B. Rowe, Aust. J. Chem., (1979), 32, 1561-1566; J. Morgan, J. Pinhey, J. Chem. Soc. Perkin Trans. 1; (1990), 3, 715-720).

In a further approach, a compound of formula (A) may be prepared by reacting a compound of formula (AN) with a compound of formula (E), wherein Hal is chlorine, bromine or iodine, in the presence of a suitable palladium catalyst (for example 0.001-50% palladium(II) acetate with respect to compound (J)) and a base (for example 1 to 10 equivalents potassium phosphate with respect to compound (J)) and preferably in the presence of a suitable ligand (for example 0.001-50% (2-dicyclohexylphosphino)-2′,4′,6′-triisopropylbiphenyl with respect to compound (J)), and in a suitable solvent (for example dioxane), preferably between 25° C. and 200° C. and optionally under microwave heating. Similar couplings are known in the literature (see for example, S. Buchwald et al., J. Am. Chem. Soc. (2000), 122, 1360-1370; B. Hong et al. WO 2005/000233).

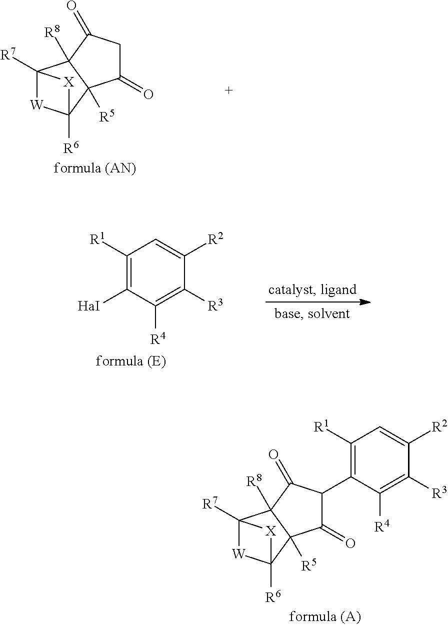

Alternatively, a compound of formula (A) may be prepared by reacting a compound of formula (AN) with a compound of formula (E), where Hal is bromine or iodine, in the presence of a suitable copper catalyst (for example 0.001-50% copper(I) iodide with respect to compound (AN)) and a base (for example 1 to 10 equivalents cesium carbonate with respect to compound (J)) and preferably in the presence of a suitable ligand (for example 0.001-50% L-proline with respect to compound (AN)), and in a suitable solvent (for example dimethylsulfoxide), preferably between 25° C. and 200° C. Similar couplings are known in the literature (see for example, Y. Jiang et al., Synlett, (2005), 18, 2731-2734, X. Xie et al., Organic Letters (2005), 7(21), 4693-4695).

In a further approach, a compound of formula (A) may be prepared by the cyclisation of a compound of formula (AS), wherein R″ is hydrogen or an alkyl group, preferably in the presence of an acid or base, and optionally in the presence of a suitable solvent, by analogous methods to those described by T. Wheeler, U.S. Pat. No. 4,283,348. A compound of formula (AS) wherein R″ is hydrogen may be cyclised under acidic conditions, preferably in the presence of a strong acid such as sulfuric acid, polyphosphoric acid or Eaton's reagent, optionally in the presence of a suitable solvent such as acetic acid, toluene or dichloromethane.

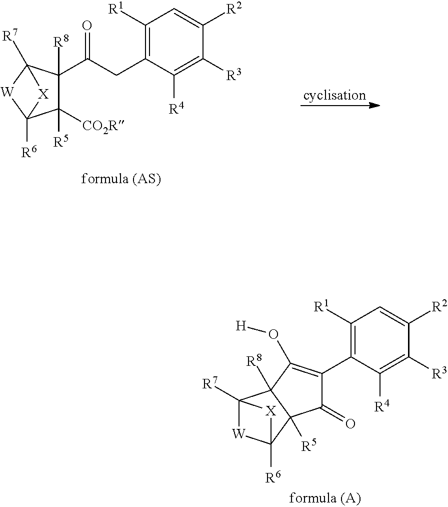

A compound of formula (AS) wherein R″ is alkyl (preferably methyl or ethyl), may be cyclised under acidic or basic conditions, preferably in the presence of at least one equivalent of a strong base such as potassium tert-butoxide, lithium diisopropylamide or sodium hydride and in a solvent such as tetrahydrofuran, dimethylsulfoxide or N,N-dimethylformamide.

A compound of formula (AS), wherein R″ is H, may be prepared by hydrolysis of a compound of formula (AT) wherein R′″ is alkyl (preferably methyl or ethyl), under standard conditions, followed by acidification of the reaction mixture to effect decarboxylation, by similar processes to those described, for example, by T. Wheeler, U.S. Pat. No. 4,283,348.

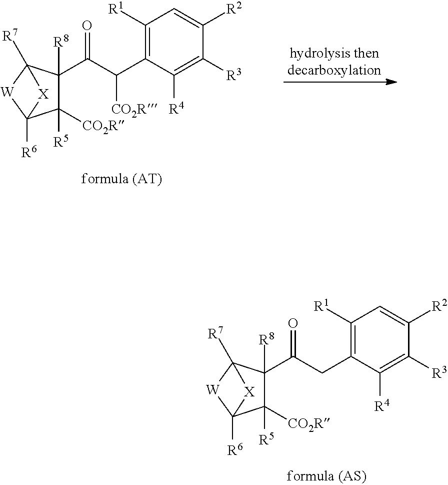

A compound of formula (AS), wherein R″ is H, may be esterified to a compound of formula (AS), wherein R″ is alkyl, under known conditions, for example by heating with an alkyl alcohol, R″OH, in the presence of an acid catalyst.

A compound of formula (AT), wherein R″ is alkyl, may be prepared by treating a compound of formula (AU) with a suitable carboxylic acid chloride of formula (AV) under basic conditions. Suitable bases include potassium tert-butoxide, sodium bis(trimethylsilyl)amide and lithium diisopropylamide and the reaction is preferably conducted in a suitable solvent (such as tetrahydrofuran or toluene) at a temperature of between −80° C. and 30° C. Alternatively, a compound of formula (AT), wherein R″ is H, may be prepared by treating a compound of formula (AU) with a suitable base (such as potassium tert-butoxide, sodium bis(trimethylsilyl)amide and lithium diisopropylamide) in a suitable solvent (such as tetrahydrofuran or toluene) at a suitable temperature (between −80° C. and 30° C.) and reacting the resulting anion with a suitable anhydride of formula (AW):

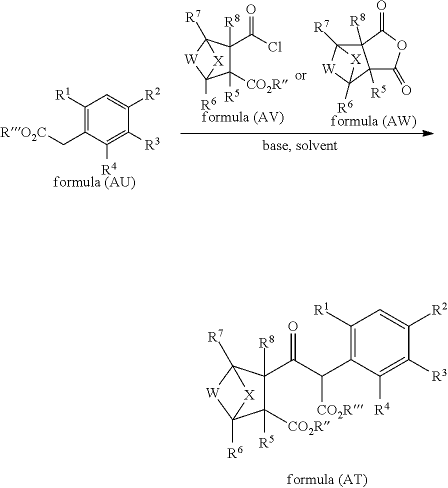

Compounds of formula (AU) are known compounds, or may be prepared from known compounds by known methods (see, for example, R. Fischer et al., WO2004/111042; T. Maetzke, S. Wendeborn and A. Stoller, WO2001/017973; F. Lieb et al., WO99/55673; F. Lieb et al., WO99/043649; I. Bell et al., GB 2326639; JP56125338 and JP56135339 (to Nippon Shinyaku Co. Ltd.); Y. Tamura et al., J. Med. Chem., (1981), 24 (8), 1006-1010).

A compound of formula (AV) may be prepared from a compound of formula (AW) by treatment with an alkyl alcohol, R″—OH, in the presence of a base, such as an alkaline metal alkoxide, or in the presence of an acid (see, for example, C. Bolm et al., J. Org. Chem., (2000), 65 (21), 6984-6991; Y. Ouzumi et al., Tetrahedron Lett., (2001), 42 (3), 411-414; D. Seebach et al., Helv. Chim. Acta, (1996), 79 (6), 1710-1740; R Aitken and J. Gospel, Tetrahedron Asymmetry, (1990), 1, 517), followed by treatment of the resulting acid with a chlorinating reagent such as oxalyl chloride or thionyl chloride under known conditions (see, for example, C. Santelli-Rouvier. Tetrahedron Lett., (1984), 25 (39), 4371; D. Walba and M. Wand, Tetrahedron Lett., (1982), 23 (48), 4995; J. Cason, Org. Synth. Coll. Vol. III, (1955), 169).

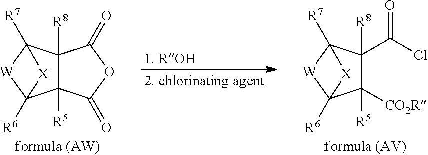

A compound of formula (AX), which is a compound of formula (AW) wherein W is an optionally substituted ethylene group, —CHR⁹—CHR¹⁰— may be prepared by the reduction of a compound of formula (AY) under known conditions (see, for example, F. Csende and G. Stajer, Org. Prep. Proceed. Int. (1999), 31 (2), 220-222).

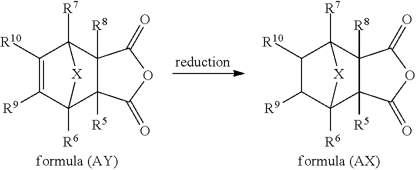

A compound of formula (AY) may be prepared by reacting a compound of formula (D) with a maleic anhydride of formula (AZ), optionally in the presence of a Lewis acid catalyst, according to procedures described, for example, by B.-C. Hong, Org. Lett., (2002), 4 (4), 663-666; S. Kobayashi et al., J. Organomet. Chem., (2001); 624 (1), 392-394; C. Song et al., Chem. Commun., (2001), 12, 1122-1123; M. De La Torre et al., Tetrahedron (1999), 55 (28), 8547-8554; J. Macauley and A. Fallis, J. Am. Chem. Soc., (1998), 110 (12), 4074-4076; S. Handy et al., Synlett., (1995), 565-567; B. Pandey and P. Dalvi, Angew. Chem. (1993), 105 (11), 1724-1726; M. Mangnus and B. Zwanenburg, Synth. Commun., (1992), 22 (5) 783-786; T.-L. Ho et al., Can. J. Chem., (1992), 70 (5), 1375-1384; P. Chemer et al., J. Org. Chem., (1992), 57 (22), 5959-5962; P. Chemer et al., J. Org. Chem., (1990), 55 (14), 4333-4337; P. Camps et al., Tetrahedron, (1984), 40, 5235-5242; G. Rubottom and D. Krueger, Tetrahedron Lett., (1977), 7, 611-614, and references therein.

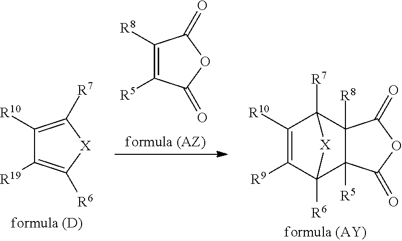

Compounds of formula (AZ) are known compounds, or may be made from known compounds by known methods.

In a further approach, a compound of formula (AY) may be prepared by reacting a compound of formula (D) with a compound of formula (BA), wherein R″″ is hydrogen or an alkyl group, under known conditions (see, for example, B.-C. Hong, Org. Lett., (2002), 4 (4), 663-666; A. Orita et al., Synlett., (2000), 5, 599-602; M. Avalos et al., Tetrahedron Lett., (1998), 39 (14), 2013-2016; M. Korzenski and J, Kolis, Tetrahedron Lett., (1997), 38 (32), 5611-5614; G. Manickam and G. Sundararajan, Indian J. Chem., Sect. B, (1996), 35 (10), 1006-1011; K. Maruoka et al., J. Am. Chem. Soc., (1994), 116 (14); 6153-6158; R. Pagni et al., Tetrahedron, (1993), 49 (31), 6743-6756; K. Rao et al., Tetrahedron Lett., (1990), 31 (41), 5959-5960) to give a compound of formula (BB), and cyclising a compound of formula (BB) under known conditions, as described, for example, by P. Camps et al., Org. Lett., (2000); 2 (26), 4225-4228; S. Sarkar and S. Ghosh, Tetrahedron, (1994), 50 (3), 921-930; D. Villemin et al., Synth. Commun., 23(4) 419-424; A. Weisz and A. Mandelbaum, J. Org. Chem., (1988), 53 (25), 5812-5815, N. Arts et al., Tetrahedron (1983), 39 (17), 2825-2830, and references therein.

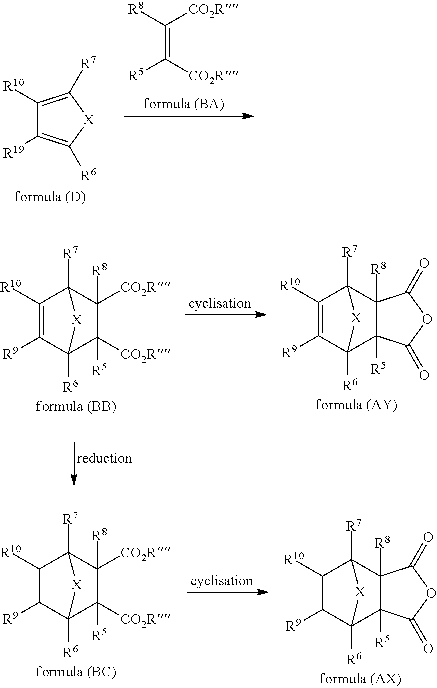

A compound of formula (BB) may also be reduced to a compound of formula (BC), and a compound of formula (BC) cyclised to a compound of formula (AX), under conditions similar to those described previously.

Compounds of formula (BA) are known compounds, or may be prepared from known compounds by known methods.

The compounds of the formulae (S), (W), (AE) and (AK) are novel and have been especially designed as intermediates for the preparation of the compounds of the formula (I).

The compounds of formula I according to the invention can be used as crop protection agents in unmodified form, as obtained in the synthesis, but they are generally formulated into crop protection compositions in a variety of ways using formulation adjuvants, such as carriers, solvents and surface-active substances. The formulations can be in various physical forms, for example in the form of dusting powders, gels, wettable powders, coated or impregnated granules for manual or mechanical distribution on target sites, water-dispersible granules, water-soluble granules, emulsifiable granules, water-dispersible tablets, effervescent compressed tablets, water-soluble tapes, emulsifiable concentrates, microemulsifiable concentrates, oil-in-water (EW) or water-in-oil (WO) emulsions, other multiphase systems such as oil/water/oil and water/oil/water products, oil flowables, aqueous dispersions, oily dispersions, suspoemulsions, capsule suspensions, soluble liquids, water-soluble concentrates (with water or a water-miscible organic solvent as carrier), impregnated polymer films or in other forms known, for example, from the Manual on Development and Use of FAO Specifications for Plant Protection Products, 5th Edition, 1999. The active ingredient may be incorporated into microfibers or micro-rods formed of polymers or polymerizable monomers and having diameter of about 0.1 to about 50 microns and aspect ratio of between about 10 and about 1000.

Such formulations can either be used directly or are diluted prior to use. They can then be applied through suitable ground or aerial application spray equipment or other ground application equipment such as central pivot irrigation systems or drip/trickle irrigation means.

Diluted formulations can be prepared, for example, with water, liquid fertilisers, micronutrients, biological organisms, oil or solvents.

The formulations can be prepared, for example, by mixing the active ingredient with formulation adjuvants in order to obtain compositions in the form of finely divided solids, granules, solutions, dispersions or emulsions. The active ingredients can also be contained in fine microcapsules consisting of a core and a polymeric shell. Microcapsules usually have a diameter of from 0.1 to 500 microns. They contain active ingredients in an amount of about from 25 to 95% by weight of the capsule weight. The active ingredients can be present in the form of liquid technical material, in the form of a suitable solution, in the form of fine particles in solid or liquid dispersion or as a monolithic solid. The encapsulating membranes comprise, for example, natural and synthetic gums, cellulose, styrene-butadiene copolymers or other similar suitable membrane forming material, polyacrylonitrile, polyacrylate, polyester, polyamides, polyureas, polyurethane, aminoplast resins or chemically modified starch or other polymers that are known to the person skilled in the art in this connection.

Alternatively it is possible for fine so called “microcapsules” to be formed wherein the active ingredient is present in the form of finely divided particles in a solid matrix of a base substance, but in that case the microcapsule is not encapsulated with a diffusion limiting membrane as outlined in the preceding paragraph.

The active ingredients may be adsorbed on a porous carrier. This may enable the active ingredients to be released into their surroundings in controlled amounts (e.g. slow release). Other forms of controlled release formulations are granules or powders in which the active ingredient is dispersed or dissolved in a solid matrix consisting of a polymer, a wax or a suitable solid substance of lower molecular weight. Suitable polymers are polyvinyl acetates, polystyrenes, polyolefins, polyvinyl alcohols, polyvinyl pyrrolidones, alkylated polyvinyl pyrrolidones, copolymers of polyvinyl pyrrolidones and maleic anhydride and esters and half-esters thereof, chemically modified cellulose esters like carboxymethyl cellulose, methyl cellulose, hydroxyethyl cellulose, examples of suitable waxes are polyethylene wax, oxidized polyethylene wax, ester waxes like montan waxes, waxes of natural origin like carnauba wax, candelilla wax, bees wax etc.

Other suitable matrix materials for slow release formulations are starch, stearin, lignin.

The formulation adjuvants suitable for the preparation of the compositions according to the invention are known per se.

As liquid carriers there may be used: water, aromatic solvents such as toluene, m-xylene, o-xylene, p-xylene and mixtures thereof, cumene, aromatic hydrocarbon blends with boiling ranges between 140 and 320° C. known under various trademarks like Solvesso®, Shellsol A®, Caromax®, Hydrosol®, paraffinic and isoparaffinic carriers such as paraffin oils, mineral oils, de-aromatized hydrocarbon solvents with boiling ranges between 50 and 320° C. known for instance under the trademark Exxsol®, non-dearomatized hydrocarbon solvents with boiling ranges between 100 and 320° C. known under the tradename Varsol®, isoparaffinic solvents with boiling ranges between 100 and 320° C. known under tradenames like Isopar® or Shellsol T®, hydrocarbons such as cyclohexane, tetrahydronaphthalene (tetralin), decahydronaphthalene, alpha-pinene, d-limonene, hexadecane, isooctane, ester solvents such as ethyl acetate, n/1-butyl acetate, amyl acetate, i-bornyl acetate, 2-ethylhexyl acetate, C₆-C₁₈ alkyl esters of acetic acid known under the tradename Exxate®, lactic acid ethylester, lactic acid propylester, lactic acid butylester, benzyl benzoate, benzyl lactate, dipropyleneglycol dibenzoate, dialkyl esters of succinic, maleic and fumaric acid and polar solvents like N-methylpyrrolidone, N-ethyl pyrrolidone, C₃-C₁₈-alkyl pyrrolidones, gamma-butyrolactone, dimethylsulfoxide, N,N-dimethyl-formamide, N,N-dimethylacetamide, N,N-dimethyllactamide, C₄-C₁₈ fatty acid dimethylamides, benzoic acid dimethylamide, acetonitrile, acetone, methyl ethyl ketone, methyl-isobutyl ketone, isoamyl ketone, 2-heptanone, cyclohexanone, isophorone, methyl isobutenyl ketone (mesityl oxide), acetophenone, ethylene carbonate, propylene carbonate, butylene carbonate, alcoholic solvents and diluents such as methanol, ethanol, propanol, n/iso-butanol, n/iso-pentanol, 2-ethyl hexanol, n-octanol, tetrahydrofurfuryl alkohol, 2-methyl-2,4-pentanediol, 4-hydroxy-4-methyl-2-pentanon, cyclohexanol, benzyl alcohol, ethylene glycol, ethylene glycol butyl ether, ethylene glycol methyl ether, diethylene glycol, diethylene glycol butyl ether, diethylene glycol ethyl ether, diethylene glycol methyl ether, propylene glycol, dipropylene glycol, dipropylene glycol methyl ether and other similar glycol ether solvents based on ethylene glycol, propylene glycol and butylene glycol feedstocks, triethylene glycol, polyethylene glycol (PEG 400), polypropylenglycols with molecular masses of 400-4000, glycerol, glycerol acetate, glycerol diacetate, glycerol triacetate, 1,4-dioxane, diethylene glycol abietate, chlorobenzene, chlorotoluene, fatty acid esters such as methyl octanoate, isopropyl myristate, methyl laurate, methyl oleate, mixture of C₈-C₁₀ fatty acid methyl esters, rape seed oil methyl and ethyl esters, soy bean oil methyl and ethyl esters, vegetable oils, fatty acids such as oleic acid, linoleic acid, linolenic acid, esters of phosphoric and phosphonic acid such as triethyl phosphate, C₃-C₁₈-tris-alkyl phosphates, alkylaryl phosphates, bis-octyl-octyl phosphonates.

Water is generally the carrier of choice for the dilution of the concentrates.

Suitable solid carriers are, for example, talc, titanium dioxide, pyrophyllite clay, silica (fumed or precipated silica and optionally functionalised or treated, for instance silanised), attapulgite clay, kieselguhr, limestone, calcium carbonate, bentonite, calcium montomorillonite, cottonseed husks, wheatmeal, soybean flour, pumice, wood flour, ground walnut shells, lignin and similar materials, as described, for example, in the EPA CFR 180.1001. (c) & (d). Powdered or granulated fertilisers can also be used as solid carriers.

A large number of surface-active substances can advantageously be used both in solid and in liquid formulations, especially in those formulations which can be diluted with a carrier prior to use. Surface-active substances may be anionic, cationic, amphoteric, non-ionic or polymeric and they may be used as emulsifiying, wetting, dispersing or suspending agents or for other purposes. Typical surface-active substances include, for example, salts of alkyl sulfates, such as diethanolammonium lauryl sulfate; Sodium lauryl sulfate, salts of alkylarylsulfonates, such as calcium or sodium dodecylbenzenesulfonate; alkylphenol-alkylene oxide addition products, such as nonylphenol ethoxylates; alcohol-alkylene oxide addition products, such as tridecyl alcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride, polyethylene glycol esters of fatty acids, such as polyethylene glycol stearate; block copolymers of ethylene oxide and propylene oxide; and salts of mono- and di-alkyl phosphate esters; and also further substances described e.g. in “McCutcheon's Detergents and Emulsifiers Annual”, MC Publishing Corp., Ridgewood, N.J., 1981.

Further adjuvants which can usually be used in pesticidal formulations include crystallisation inhibitors, viscosity-modifying substances, suspending agents, dyes, anti-oxidants, foaming agents, light absorbers, mixing aids, anti-foams, complexing agents, neutralising or pH-modifying substances and buffers, corrosion-inhibitors, fragrances, wetting agents, absorption improvers, micronutrients, plasticisers, glidants, lubricants, dispersants, thickeners, anti-freezes, microbiocides, compatibility agents and solubilisers and also liquid and solid fertilisers.

The formulations may also comprise additional active substances, for example further herbicides, herbicide safeners, plant growth regulators, fungicides or insecticides.

The compositions according to the invention can additionally include an additive (commonly referred to as an adjuvant), comprising a mineral oil, an oil of vegetable or animal origin, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsifiable vegetable oil, such as AMIGO® (Loveland Products Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is AGNIQUE ME 18 RD-F® (Cognis). Those and other oil derivatives are also known from the Compendium of Herbicide Adjuvants, 5th Edition, Southern Illinois University, 2000.

The application and action of the oil additives can be further improved by combining them with surface-active substances, such as non-ionic, anionic, cationic or amphoteric surfactants. Examples of suitable anionic, non-ionic, cationic or amphoteric surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant). Also preferred are silicone surfactants, especially polyalkyl-oxide-modified heptamethyltrisiloxanes, which are commercially available e.g. as SILWET L-77®, and also perfluorinated surfactants. The concentration of surface-active substances in relation to the total additive is generally from 1 to 50% by weight. Examples of oil additives that consist of mixtures of oils or mineral oils or derivatives thereof with surfactants are TURBOCHARGE®, ADIGOR® (both (Syngenta Crop Protection AG), ACTIPRON® (BP Oil UK Limited), AGRI-DEX® (Helena Chemical Company).

The said surface-active substances may also be used in the formulations alone, that is to say without oil additives.

Furthermore, the addition of an organic solvent to the oil additive/surfactant mixture can contribute to a further enhancement of action. Suitable solvents are, for example, SOLVESSO® and AROMATIC® solvents (Exxon Corporation). The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE® (BASF). Further oil additives that are preferred according to the invention are SCORE® and ADIGOR® (both Syngenta Crop Protection AG).

In addition to the oil additives listed above, in order to enhance the activity of the compositions according to the invention it is also possible for formulations of alkylpyrrolidones, (e.g. AGRIMAX® from ISP) to be added to the spray mixture. Formulations of synthetic latices, such as, for example, polyacrylamide, polyvinyl compounds or poly-1-p-menthene (e.g. BOND®, COURIER® or EMERALD®) can also be used.

Such adjuvant oils as described in the preceding paragraphs may be employed as the carrier liquid in which an active compound is dissolved, emulsified or dispersed as appropriate to the physical form of the active compound.

The pesticidal formulations generally contain from 0.1 to 99% by weight, especially from 0.1 to 95% by weight, of a compound of formula I and from 1 to 99.9% by weight of a formulation adjuvant, which preferably includes from 0 to 25% by weight of a surface-active substance. Whereas commercial products will preferably be formulated as concentrates, the end user will normally employ dilute formulations.

The rate of application of the compounds of formula I may vary within wide limits and depends upon the nature of the soil, the method of application (pre- or post-emergence; seed dressing; application to the seed furrow; no tillage application etc.), the crop plant, the weed or grass to be controlled, the prevailing climatic conditions, and other factors governed by the method of application, the time of application and the target crop. The compounds of formula I according to the invention are generally applied at a rate of 1-2000 g/ha, preferably 1-1000 g/ha and most preferably at 1-500 g/ha.

Preferred formulations have especially the following representative compositions: (%=percent by weight):

Emulsifiable Concentrates:

active ingredient: 1 to 95%, preferably 60 to 90%

surface-active agents: 1 to 30%, preferably 5 to 20%

solvents as liquid carrier: 1 to 80%, preferably 1 to 35%

Dusts:

active ingredient: 0.1 to 10%, preferably 0.1 to 5%

solid carriers: 99.9 to 90%, preferably 99.9 to 99%

Suspension Concentrates:

active ingredient: 5 to 75%, preferably 10 to 50%

water: 94 to 24%, preferably 88 to 30%

surface-active agents: 1 to 40%, preferably 2 to 30%

Wettable Powders:

active ingredient: 0.5 to 90%, preferably 1 to 80%

surface-active agents: 0.5 to 20%, preferably 1 to 15%

solid carriers: 5 to 95%, preferably 15 to 90%

Granules:

active ingredient: 0.1 to 30%, preferably 0.1 to 15%

solid carriers: 99.5 to 70%, preferably 97 to 85%

Water Dispersible Granules:

active ingredient: 1 to 90%, preferably 10 to 80%

surface-active agents: 0.5 to 80%, preferably 5 to 30%

solid carriers: 90 to 10%, preferably 70 to 30%

The following Examples further illustrate, but do not limit, the invention.

| F1. Emulsifiable concentrates | a) | b) | c) | d) |
|---|---|---|---|---|
| active ingredient | 5% | 10% | 25% | 50% |
| calcium dodecylbenzene- | 6% | 8% | 6% | 8% |
| sulfonate | | | | |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | 10% | | 20% |
| arom. hydrocarbon | 85% | 68% | 65% | 16% |
| mixture C₉-C₁₂ | | | | |

Emulsions of any desired concentration can be prepared from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | | | | |
| propoxy)-propane | 40% | 50% | — | |
| polyethylene glycol MW 400 | 20% | 10% | — | — |
| NMP | — | | 50% | 10% |
| arom. hydrocarbon | 35% | 30% | — | — |
| mixture C₉-C₁₂ | | | | |

The solutions are suitable for application undiluted or after dilution with water.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | | | | |
| sulfonate | — | 6% | 5% | 6% |
| octylphenol polyglycol ether | — | 1% | 2% | — |
| (7-8 mol of ethylene oxide) | | | | |
| highly disperse silicic acid | 1% | 3% | 5% | 10% |
| kaolin | 88% | 62% | 35% | — |

The active ingredient is thoroughly mixed with the adjuvants and the mixture is thoroughly ground in a suitable mill, yielding wettable powders which can be diluted with water to give suspensions of any desired concentration.

| F4. Coated granules | a) | b) | c) |
|---|---|---|---|
| active ingredient | 0.1% | 5% | 15% |
| highly dispersed silica | 0.9% | 2% | 2% |
| inorg. carrier | 99.0% | 93% | 83% |
| (diameter 0.1 - 1 mm) | | | |
| e.g. CaCO₃ or SiO₂ | | | |

The active ingredient is dissolved in methylene chloride, the solution is sprayed onto the carrier and the solvent is subsequently evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silica | 0.9% | 1% | 2% |
| inorg. carrier | 98.0% | 92% | 80% |
| (diameter 0.1 - 1 mm) | | | |
| e.g. CaCO₃ or SiO₂ | | | |

The finely ground active ingredient is applied uniformly, in a mixer, to the carrier moistened with polyethylene glycol. Non-dusty coated granules are obtained in this manner.

| F6. Extruded granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants and the mixture is moistened with water. The resulting mixture is extruded and then dried in a stream of air.

| F7. Water-dispersible granules | a) | b) | c) | d) |
|---|---|---|---|---|
| active ingredient | 5% | 10% | 40% | 90% |
| sodium lignosulfonate | 20% | 20% | 15% | 7% |
| dibutyl naphthalene sulfonate | 5% | 5% | 4% | 2% |
| Gum arabic | 2% | 1% | 1% | 1% |
| Diatomaceous earth | 20% | 30% | 5% | |
| Sodium sulfate | | 4% | 5% | |
| kaolin | 48% | 30% | 30% | |

The active ingredient is mixed and ground with the adjuvants and the mixture is moistened with water. The resulting mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| propylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 7% | 6% |
| heteropolysacharide (Xanthan) | 0.2% | 0.2% | 0.2% | 0.2% |
| 1,2-Benzisothiazolin-3-on | 0.1% | 0.1% | 0.1% | 0.1% |
| silicone oil emulsion | 0.7% | 0.7% | 0.7% | 0.7% |
| water | 87% | 79% | 62% | 38% |

The finely ground active ingredient is intimately mixed with the adjuvants, yielding a suspension concentrate from which suspensions of any desired concentration can be prepared by dilution with water.

Crops of useful plants in which the compositions according to the invention can be used include especially cereals, in particular wheat and barley, rice, corn, rape, sugarbeet, sugarcane, soybean, cotton, sunflower, peanut and plantation crops.

The term “crops” is to be understood as also including crops that have been rendered tolerant to herbicides or classes of herbicides (for example ALS, GS, EPSPS, PPO and HPPD inhibitors) as a result of conventional methods of breeding or genetic engineering. An example of a crop that has been rendered tolerant e.g. to imidazolinones, such as imazamox, by conventional methods of breeding is Clearfield® summer rape (Canola). Examples of crops that have been rendered tolerant to herbicides by genetic engineering methods include e.g. glyphosate- and glufosinate-resistant maize varieties commercially available under the trade names RoundupReady® and LibertyLink®. The weeds to be controlled may be both monocotyledonous and dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus, Abutilon, Sida, Xanthium, Amaranthus, Chenopodium, Ipomoea, Chrysanthemum, Galium, Viola and Veronica. Control of monocotyledonous weeds, in particular Agrostis, Avena, Setaria, Lolium, Echinochloa, Bromus, Alopecurus and Sorghum is very extensive.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt-176 maize hybrids of NK® (Syngenta Seeds). The Bt toxin is a protein that is formed naturally by Bacillus thuringiensis soil bacteria. Examples of toxins and transgenic plants able to synthesise such toxins are described in EP-A-451 878, EP-A-374 753, WO 93/07278, WO 95/34656, WO 03/052073 and EP-A-427 529. Examples of transgenic plants that contain one or more genes which code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops and their seed material can be resistant to herbicides and at the same time also to insect feeding (“stacked” transgenic events). Seed can, for example, have the ability to express an insecticidally active Cry3 protein and at the same time be glyphosate-tolerant. The term “crops” is to be understood as also including crops obtained as a result of conventional methods of breeding or genetic engineering which contain so-called output traits (e.g. improved flavour, storage stability, nutritional content).

Areas under cultivation are to be understood as including land where the crop plants are already growing as well as land intended for the cultivation of those crop plants.

The compounds of formula I according to the invention can also be used in combination with further herbicides. Preferably, in these mixtures, the compound of the formula I is one of those compounds listed in Tables 1 to 24 below. The following mixtures of the compound of formula I are especially important:

compound of formula I+acetochlor, compound of formula I+acifluorfen, compound of formula I+acifluorfen-sodium, compound of formula I+aclonifen, compound of formula I+acrolein, compound of formula I+alachlor, compound of formula I+alloxydim, compound of formula I+allyl alcohol, compound of formula I+ametryn, compound of formula I+amicarbazone, compound of formula I+amidosulfuron, compound of formula I+aminopyralid, compound of formula I+amitrole, compound of formula I+ammonium sulfamate, compound of formula I+anilofos, compound of formula I+asulam, compound of formula I+atraton, compound of formula I+atrazine, compound of formula I+azimsulfuron, compound of formula I+BCPC, compound of formula I+beflubutamid, compound of formula I+benazolin, compound of formula I+benfluralin, compound of formula I+benfuresate, compound of formula I+bensulfuron, compound of formula I+bensulfuron-methyl, compound of formula I+bensulide, compound of formula I+bentazone, compound of formula I+benzfendizone, compound of formula I+benzobicyclon, compound of formula I+benzofenap, compound of formula I+bifenox, compound of formula I+bilanafos, compound of formula I+bispyribac, compound of formula I+bispyribac-sodium, compound of formula I+borax, compound of formula I+bromacil, compound of formula I+bromobutide, compound of formula I+bromoxynil, compound of formula I+butachlor, compound of formula I+butafenacil, compound of formula I+butamifos, compound of formula I+butralin, compound of formula I+butroxydim, compound of formula I+butylate, compound of formula I+cacodylic acid, compound of formula I+calcium chlorate, compound of formula I+cafenstrole, compound of formula I+carbetamide, compound of formula I+carfentrazone, compound of formula I+carfentrazone-ethyl, compound of formula I+CDEA, compound of formula I+CEPC, compound of formula I+chlorflurenol, compound of formula I+chlorflurenol-methyl, compound of formula I+chloridazon, compound of formula I+chlorimuron, compound of formula I+chlorimuron-ethyl, compound of formula I+chloroacetic acid, compound of formula I+chlorotoluron, compound of formula I+chiorpropham, compound of formula I+chlorsulfuron, compound of formula I+chlorthal, compound of formula I+chlorthal-dimethyl, compound of formula I+cinidon-ethyl, compound of formula I+cinmethylin, compound of formula I+cinosulfuron, compound of formula I+cisanilide, compound of formula I+clethodim, compound of formula I+clodinafop, compound of formula I+clodinafop-propargyl, compound of formula I+clomazone, compound of formula I+clomeprop, compound of formula I+clopyralid, compound of formula I+cloransulam, compound of formula I+cloransulam-methyl, compound of formula I+CMA, compound of formula I+4-CPB, compound of formula I+CPMF, compound of formula I+4-CPP, compound of formula I+CPPC, compound of formula I+cresol, compound of formula I+cumyluron, compound of formula I+cyanamide, compound of formula I+cyanazine, compound of formula I+cycloate, compound of formula I+cyclosulfamuron, compound of formula I+cycloxydim, compound of formula I+cyhalofop, compound of formula I+cyhalofop-butyl, compound of formula I+2,4-D, compound of formula I+3,4-DA, compound of formula I+daimuron, compound of formula I+dalapon, compound of formula I+dazomet, compound of formula I+2,4-DB, compound of formula I+3,4-DB, compound of formula I+2,4-DEB, compound of formula I+desmedipham, compound of formula I+dicamba, compound of formula I+dichlobenil, compound of formula I+ortho-dichlorobenzene, compound of formula I+para-dichlorobenzene, compound of formula I+dichlorprop, compound of formula I+dichlorprop-P, compound of formula I+diclofop, compound of formula I+diclofop-methyl, compound of formula I+diclosulam, compound of formula I+difenzoquat, compound of formula I+difenzoquat metilsulfate, compound of formula I+diflufenican, compound of formula I+diflufenzopyr, compound of formula I+dimefuron, compound of formula I+dimepiperate, compound of formula I+dimethachlor, compound of formula I+dimethametryn, compound of formula I+dimethenamid, compound of formula I+dimethenamid-P, compound of formula I+dimethipin, compound of formula I+dimethylarsinic acid, compound of formula I+dinitramine, compound of formula I+dinoterb, compound of formula I+diphenamid, compound of formula I+diquat, compound of formula I+diquat dibromide, compound of formula I+dithiopyr, compound of formula I+diuron, compound of formula I+DNOC, compound of formula I+3,4-DP, compound of formula I+DSMA, compound of formula I+EBEP, compound of formula I+endothal, compound of formula I+EPTC, compound of formula I+esprocarb, compound of formula I+ethalfluralin, compound of formula I+ethametsulfuron, compound of formula I+ethametsulfuron-methyl, compound of formula I+ethofumesate, compound of formula I+ethoxyfen, compound of formula I+ethoxysulfuron, compound of formula I+etobenzanid, compound of formula I+fenoxaprop-P, compound of formula I+fenoxaprop-P-ethyl, compound of formula I+fentrazamide, compound of formula I+ferrous sulfate, compound of formula I+flamprop-M, compound of formula I+flazasulfuron, compound of formula I+florasulam, compound of formula I+fluazifop, compound of formula I+fluazifop-butyl, compound of formula I+fluazifop-P, compound of formula I+fluazifop-P-butyl, compound of formula I+flucarbazone, compound of formula I+flucarbazone-sodium, compound of formula I+flucetosulfuron, compound of formula I+fluchloralin, compound of formula I+flufenacet, compound of formula I+flufenpyr, compound of formula I+flufenpyr-ethyl, compound of formula I+flumetsulam, compound of formula I+flumiclorac, compound of formula I+flumiclorac-pentyl, compound of formula I+flumioxazin, compound of formula I+fluometuron, compound of formula I+fluoroglycofen, compound of formula I+fluoroglycofen-ethyl, compound of formula I+flupropanate, compound of formula I+flupyrsulfuron, compound of formula I+flupyrsulfuron-methyl-sodium, compound of formula I+flurenol, compound of formula I+fluridone, compound of formula I+fluorochloridone, compound of formula I+fluoroxypyr, compound of formula I+flurtamone, compound of formula I+fluthiacet, compound of formula I+fluthiacet-methyl, compound of formula I+fomesafen, compound of formula I+foramsulfuron, compound of formula I+fosamine, compound of formula I+glufosinate, compound of formula I+glufosinate-ammonium, compound of formula I+glyphosate, compound of formula I+halosulfuron, compound of formula I+halosulfuron-methyl, compound of formula I+haloxyfop, compound of formula I+haloxyfop-P, compound of formula I+HC-252, compound of formula I+hexazinone, compound of formula I+imazamethabenz, compound of formula I+imazamethabenz-methyl, compound of formula I+imazamox, compound of formula I+imazapic, compound of formula I+imazapyr, compound of formula I+imazaquin, compound of formula I+imazethapyr, compound of formula I+imazosulfuron, compound of formula I+indanofan, compound of formula I+iodomethane, compound of formula I+iodosulfuron, compound of formula I+iodosulfuron-methyl-sodium, compound of formula I+ioxynil, compound of formula I+isoproturon, compound of formula I+isouron, compound of formula I+isoxaben, compound of formula I+isoxachlortole, compound of formula I+isoxaflutole, compound of formula I+karbutilate, compound of formula I+lactofen, compound of formula I+lenacil, compound of formula I+linuron, compound of formula I+MAA, compound of formula I+MAMA, compound of formula I+MCPA, compound of formula I+MCPA-thioethyl, compound of formula I+MCPB, compound of formula I+mecoprop, compound of formula I+mecoprop-P, compound of formula I+mefenacet, compound of formula I+mefluidide, compound of formula I+mesosulfuron, compound of formula I+mesosulfuron-methyl, compound of formula I+mesotrione, compound of formula I+metam, compound of formula I+metamifop, compound of formula I+metamitron, compound of formula I+metazachlor, compound of formula I+methabenzthiazuron, compound of formula I+methylarsonic acid, compound of formula I+methyldymron, compound of formula I+methyl isothiocyanate, compound of formula I+metobenzuron, compound of formula I+metolachlor, compound of formula I+S-metolachlor, compound of formula I+metosulam, compound of formula I+metoxuron, compound of formula I+metribuzin, compound of formula I+metsulfuron, compound of formula I+metsulfuron-methyl, compound of formula I+MK-616, compound of formula I+molinate, compound of formula I+monolinuron, compound of formula I+MSMA, compound of formula I+naproanilide, compound of formula I+napropamide, compound of formula I+naptalam, compound of formula I+neburon, compound of formula I+nicosulfuron, compound of formula I+nonanoic acid, compound of formula I+norflurazon, compound of formula I+oleic acid (fatty acids), compound of formula I+orbencarb, compound of formula I+orthosulfamuron, compound of formula I+oryzalin, compound of formula I+oxadiargyl, compound of formula I+oxadiazon, compound of formula I+oxasulfuron, compound of formula I+oxaziclomefone, compound of formula I+oxyfluorfen, compound of formula I+paraquat, compound of formula I+paraquat dichloride, compound of formula I+pebulate, compound of formula I+pendimethalin, compound of formula I+penoxsulam, compound of formula I+pentachlorophenol, compound of formula I+pentanochlor, compound of formula I+pentoxazone, compound of formula I+pethoxamid, compound of formula I+petrolium oils, compound of formula I+phenmedipham, compound of formula I+phenmedipham-ethyl, compound of formula I+picloram, compound of formula I+picolinafen, compound of formula I+pinoxaden, compound of formula I+piperophos, compound of formula I+potassium arsenite, compound of formula I+potassium azide, compound of formula I+pretilachlor, compound of formula I+primisulfuron, compound of formula I+primisulfuron-methyl, compound of formula I+prodiamine, compound of formula I+profluazol, compound of formula I+profoxydim, compound of formula I+prometon, compound of formula I+prometryn, compound of formula I+propachlor, compound of formula I+propanil, compound of formula I+propaquizafop, compound of formula I+propazine, compound of formula I+propham, compound of formula I+propisochlor, compound of formula I+propoxycarbazone, compound of formula I+propoxycarbazone-sodium, compound of formula I+propyzamide, compound of formula I+prosulfocarb, compound of formula I+prosulfuron, compound of formula I+pyraclonil, compound of formula I+pyraflufen, compound of formula I+pyraflufen-ethyl, compound of formula I+pyrazolynate, compound of formula I+pyrazosulfuron, compound of formula I+pyrazosulfuron-ethyl, compound of formula I+pyrazoxyfen, compound of formula I+pyribenzoxim, compound of formula I+pyributicarb, compound of formula I+pyridafol, compound of formula I+pyridate, compound of formula I+pyriftalid, compound of formula I+pyriminobac, compound of formula I+pyriminobac-methyl, compound of formula I+pyrimisulfan, compound of formula I+pyrithiobac, compound of formula I+pyrithiobac-sodium, compound of formula I+quinclorac, compound of formula I+quinmerac, compound of formula I+quinoclamine, compound of formula I+quizalofop, compound of formula I+quizalofop-P, compound of formula I+rimsulfuron, compound of formula I+sethoxydim, compound of formula I+siduron, compound of formula I+simazine, compound of formula I+simetryn, compound of formula I+SMA, compound of formula I+sodium arsenite, compound of formula I+sodium azide, compound of formula I+sodium chlorate, compound of formula I+sulcotrione, compound of formula I+sulfentrazone, compound of formula I+sulfometuron, compound of formula I+sulfometuron-methyl, compound of formula I+sulfosate, compound of formula I+sulfosulfuron, compound of formula I+sulfuric acid, compound of formula I+tar oils, compound of formula I∓2,3,6-TBA, compound of formula I+TCA, compound of formula I+TCA-sodium, compound of formula I+tebuthiuron, compound of formula I+tepraloxydim, compound of formula I+terbacil, compound of formula I+terbumeton, compound of formula I+terbuthylazine, compound of formula I+terbutryn, compound of formula I+thenylchlor, compound of formula I+thiazopyr, compound of formula I+thifensulfuron, compound of formula I+thifensulfuron-methyl, compound of formula I+thiobencarb, compound of formula I+tiocarbazil, compound of formula I+topramezone, compound of formula I+tralkoxydim, compound of formula I+tri-allate, compound of formula I+triasulfuron, compound of formula I+triaziflam, compound of formula I+tribenuron, compound of formula I+tribenuron-methyl, compound of formula I+tricamba, compound of formula I+triclopyr, compound of formula I+trietazine, compound of formula I+trifloxysulfuron, compound of formula I+trifloxysulfuron-sodium, compound of formula I+trifluralin, compound of formula I+triflusulfuron, compound of formula I+triflusulfuron-methyl, compound of formula I+trihydroxytriazine, compound of formula I+tritosulfuron, compound of formula I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula I+4-[(4,5-dihydro-3-methoxy-4-methyl-5-oxo)-1H-1,2,4-triazol-1-ylcarbonylsulfamoyl]-5-methylthiophene-3-carboxylic acid (BAY636), compound of formula I+BAY747 (CAS RN 335104-84-2), compound of formula I+topramezone (CAS RN 210631-68-8), compound of formula I+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), and compound of formula I+4-hydroxy-3-[[2-(3-methoxypropyl)-6-(difluoromethyl)-3-pyridinyl]carbonyl]-bicyclo[3.2.1]oct-3-en-2-one.

The mixing partners for the compound of formula I may also be in the form of esters or salts, as mentioned e.g. in The Pesticide Manual, 12th Edition (BCPC) 2000.

For applications in cereals, the following mixtures are preferred: compound of formula I+aclonifen, compound of formula I+amidosulfuron, compound of formula I+aminopyralid, compound of formula I+beflubutamid, compound of formula I+benfluralin, compound of formula I+bifenox, compound of formula I+bromoxynil, compound of formula I+butafenacil, compound of formula I+carbetamide, compound of formula I+carfentrazone, compound of formula I+carfentrazone-ethyl, compound of formula I+chlorotoluron, compound of formula I+chlorpropham, compound of formula I+chlorsulfuron, compound of formula I+cinidon-ethyl, compound of formula I+clodinafop, compound of formula I+clodinafop-propargyl, compound of formula I+clopyralid, compound of formula I+2,4-D, compound of formula I+dicamba, compound of formula I+dichlobenil, compound of formula I+dichlorprop, compound of formula I+diclofop, compound of formula I+diclofop-methyl, compound of formula I+difenzoquat, compound of formula I+difenzoquat metilsulfate, compound of formula I+diflufenican, compound of formula I+diquat, compound of formula I+diquat dibromide, compound of formula I+fenoxaprop-P, compound of formula I+fenoxaprop-P-ethyl, compound of formula I+flamprop-M, compound of formula I+florasulam, compound of formula I+fluazifop-P-butyl, compound of formula I+flucarbazone, compound of formula I+flucarbazone-sodium, compound of formula I+flufenacet, compound of formula I+flupyrsulfuron, compound of formula I+flupyrsulfuron-methyl-sodium, compound of formula I+fluorochloridone, compound of formula I+fluoroxypyr, compound of formula I+flurtamone, compound of formula I+imazamethabenz-methyl, compound of formula I+imazamox, compound of formula I+iodosulfuron, compound of formula I+iodosulfuron-methyl-sodium, compound of formula I+ioxynil, compound of formula I+isoproturon, compound of formula I+linuron, compound of formula I+MCPA, compound of formula I+mecoprop, compound of formula I+mecoprop-P, compound of formula I+mesosulfuron, compound of formula I+mesosulfuron-methyl, compound of formula I+mesotrione, compound of formula I+metribuzin, compound of formula I+metsulfuron, compound of formula I+metsulfuron-methyl, compound of formula I+pendimethalin, compound of formula I+picolinafen, compound of formula I+pinoxaden, compound of formula I+prodiamine, compound of formula I+propanil, compound of formula I+propoxycarbazone, compound of formula I+propoxycarbazone-sodium, compound of formula I+prosulfocarb, compound of formula I+pyrasulfotole, compound of formula I+pyridate, compound of formula I+pyroxasulfone (KIN-485), compound of formula I+pyroxsulam compound of formula I+sulfosulfuron, compound of formula 1+tembotrione, compound of formula I+terbutryn, compound of formula I+thifensulfuron, compound of formula I+thiencarbazone, compound of formula I+thifensulfuron-methyl, compound of formula I+topramezone, compound of formula I+tralkoxydim, compound of formula I+tri-allate, compound of formula I+triasulfuron, compound of formula I+tribenuron, compound of formula I+tribenuron-methyl, compound of formula I+trifluralin, compound of formula I+trinexapac-ethyl and compound of formula I+tritosulfuron, where

the mixtures comprising a compound of formula (I)+amidosulfuron, compound of formula (I)+aminopyralid, compound of formula (I)+beflubutamid, compound of formula (I)+bromoxynil, compound of formula (I)+carfentrazone, compound of formula (I)+carfentrazone-ethyl, compound of formula (I)+chlorotoluron, compound of formula (I)+chlorsulfuron, compound of formula (I)+clodinafop, compound of formula (I)+clodinafop-propargyl, compound of formula (I)+clopyralid, 2,4-D, compound of formula (I)+dicamba, compound of formula (I)+difenzoquat, compound of formula (I)+difenzoquat metilsulfate, compound of formula (I)+diflufenican, compound of formula (I)+fenoxaprop-P, compound of formula (I)+fenoxaprop-P-ethyl, compound of formula (I)+florasulam, compound of formula (I)+flucarbazone, compound of formula (I)+flucarbazone-sodium, compound of formula (I)+flufenacet, compound of formula (I)+flupyrsulfuron, compound of formula (I)+flupyrsulfuron-methyl-sodium, compound of formula (I)+fluoroxypyr, compound of formula (I)+flurtamone, compound of formula (I)+iodosulfuron, compound of formula (I)+iodosulfuron-methyl-sodium, compound of formula (I)+MCPA, compound of formula (I)+mesosulfuron, compound of formula (I)+mesosulfuron-methyl, compound of formula (I)+metsulfuron, compound of formula (I)+metsulfuron-methyl, compound of formula (I)+pendimethalin, compound of formula (I)+picolinafen, compound of formula (I)+pinoxaden, compound of formula (I)+prosulfocarb, compound of formula (I)+pyrasulfotole, compound of formula (I)+pyroxasulfone (KIN-485), compound of formula (I)+pyroxsulam, compound of formula (I)+sulfosulfuron, compound of formula (I)+thifensulfuron, compound of formula (I)+thifensulfuron-methyl, compound of formula (I)+tralkoxydim, compound of formula (I)+triasulfuron, compound of formula (I)+tribenuron, compound of formula (I)+tribenuron-methyl, compound of formula (I)+trifluralin, compound of formula (I)+trinexapac-ethyl and compound of formula (I)+tritosulfuron are particularly preferred.

For applications in rice, the following mixtures are preferred: compound of formula (I)+azimsulfuron, compound of formula (I)+bensulfuron, compound of formula (I)+bensulfuron-methyl, compound of formula (I)+benzobicyclon, compound of formula (I)+benzofenap, compound of formula (I)+bispyribac, compound of formula (I)+bispyribac-sodium, compound of formula (I)+butachior, compound of formula (I)+cafenstrole, compound of formula (I)+cinosulfuron, compound of formula (I)+clomazone, compound of formula (I)+clomeprop, compound of formula (I)+cyclosulfamuron, compound of formula (I)+cyhalofop, compound of formula (I)+cyhalofop-butyl, compound of formula (I)+2,4-D, compound of formula (I)+daimuron, compound of formula (I)+dicamba, compound of formula (I)+diquat, compound of formula (I)+diquat dibromide, compound of formula (I)+esprocarb, compound of formula (I)+ethoxysulfuron, compound of formula (I)+fenoxaprop-P, compound of formula (I)+fenoxaprop-P-ethyl, compound of formula (I)+fentrazamide, compound of formula (I)+florasulam, compound of formula (I)+glufosinate-ammonium, compound of formula (I)+glyphosate, compound of formula (I)+halosulfuron, compound of formula (I)+halosulfuron-methyl, compound of formula (I)+imazosulfuron, compound of formula (I)+MCPA, compound of formula (I)+mefenacet, compound of formula (I)+mesotrione, compound of formula (I)+metamifop, compound of formula (I)+metsulfuron, compound of formula (I)+metsulfuron-methyl, compound of formula (I)+n-methyl glyphosate, compound of formula (I)+orthosulfamuron, compound of formula (I)+oryzalin, compound of formula (I)+oxadiargyl, compound of formula (I)+oxadiazon, compound of formula (I)+paraquat dichloride, compound of formula (I)+pendimethalin, compound of formula (I)+penoxsulam, compound of formula (I)+pretilachlor, compound of formula (I)+profoxydim, compound of formula (I)+propanil, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyriftalid, compound of formula (I)+pyriminobac, compound of formula (I)+pyriminobac-methyl, compound of formula (I)+pyrimisulfan, compound of formula (I)+quinclorac, compound of formula (I)+tefuryltrione, compound of formula (I)+triasulfuron and compound of formula (I)+trinexapac-ethyl, where the mixtures comprising a compound of formula (I)+azimsulfuron, compound of formula (I)+bensulfuron, compound of formula (I)+bensulfuron-methyl, compound of formula (I)+benzobicyclon, compound of formula (I)+benzofenap, compound of formula (I)+bispyribac, compound of formula (I)+bispyribac-sodium, compound of formula (I)+clomazone, compound of formula (I)+clomeprop, compound of formula (I)+cyhalofop, compound of formula (I)+cyhalofop-butyl, compound of formula (I)+2,4-D, compound of formula (I)+daimuron, compound of formula (I)+dicamba, compound of formula (I)+esprocarb, compound of formula (I)+ethoxysulfuron, compound of formula (I)+fenoxaprop-P, compound of formula (I)+fenoxaprop-P-ethyl, compound of formula (I)+fentrazamide, compound of formula (I)+florasulam, compound of formula (I)+halosulfuron, compound of formula (I)+halosulfuron-methyl, compound of formula (I)+imazosulfuron, compound of formula (I)+MCPA, compound of formula (I)+mefenacet, compound of formula (I)+mesotrione, compound of formula (I)+metsulfuron, compound of formula (I)+metsulfuron-methyl, compound of formula (I)+orthosulfamuron, compound of formula (I)+oxadiargyl, compound of formula (I)+oxadiazon, compound of formula (I)+pendimethalin, compound of formula (I)+penoxsulam, compound of formula (I)+pretilachior, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyriftalid, compound of formula (I)+pyriminobac, compound of formula (I)+pyriminobac-methyl, compound of formula (I)+pyrimisulfan, compound of formula (I)+quinclorac, compound of formula (I)+tefuryltrione, compound of formula (I)+triasulfuron and compound of formula (I)+trinexapac-ethyl are particularly preferred.

The compounds of formula I according to the invention can also be used in combination with safeners. Preferably, in these mixtures, the compound of the formula I is one of those compounds listed in Tables 1 to 24 below. The following mixtures with safeners, especially, come into consideration:

compound of formula I+cloquintocet-mexyl, compound of formula I+cloquintocet acid and salts thereof, compound of formula I+fenchlorazole-ethyl, compound of formula I+fenchlorazole acid and salts thereof, compound of formula I+mefenpyr-diethyl, compound of formula I+mefenpyr diacid, compound of formula I+isoxadifen-ethyl, compound of formula I+isoxadifen acid, compound of formula I+furilazole, compound of formula I+furilazole R isomer, compound of formula (I)+N-(2-methoxybenzoyl)-4-[(methylaminocarbonyl)amino]benzenesulfonamide, compound of formula I+benoxacor, compound of formula I+dichlormid, compound of formula I+AD-67, compound of formula I+oxabetrinil, compound of formula I+cyometrinil, compound of formula I+cyometrinil Z-isomer, compound of formula I+fenclorim, compound of formula I+cyprosulfamide, compound of formula I+naphthalic anhydride, compound of formula I+flurazole, compound of formula I+CL 304,415, compound of formula I+dicyclonon, compound of formula I+fluxofenim, compound of formula I+DKA-24, compound of formula I+R-29148 and compound of formula I+PPG-1292. A safening effect can also be observed for the mixtures compound of the formula I+dymron, compound of the formula I+MCPA, compound of the formula I+mecopropand compound of the formula I+mecoprop-P.

The above-mentioned safeners and herbicides are described, for example, in the Pesticide Manual, Twelfth Edition, British Crop Protection Council, 2000. R-29148 is described, for example by P. B. Goldsbrough et al., Plant Physiology, (2002), Vol. 130 pp. 1497-1505 and references therein, PPG-1292 is known from WO09211761 and N-(2-methoxybenzoyl)-4-[(methylaminocarbonyl)amino]benzenesulfonamide is known from EP365484.

Benoxacor, cloquintocet-mexyl, cyprosulfamide, mefenpyr-diethyl and N-(2-methoxybenzoyl)-4-[(methylaminocarbonyl)amino]benzenesulfonamide are especially preferred, where cloquintocet-mexyl is particularly valuable.

The rate of application of safener relative to the herbicide is largely dependent upon the mode of application. In the case of field treatment, generally from 0.001 to 5.0 kg of safener/ha, preferably from 0.001 to 0.5 kg of safener/ha, and generally from 0.001 to 2 kg of herbicide/ha, but preferably from 0.005 to 1 kg/ha, are applied.

The herbicidal compositions according to the invention are suitable for all methods of application customary in agriculture, such as, for example, pre-emergence application, post-emergence application and seed dressing. Depending upon the intended use, the safeners can be used for pretreating the seed material of the crop plant (dressing the seed or seedlings) or introduced into the soil before or after sowing, followed by the application of the (unsafened) compound of the formula (I), optionally in combination with a co-herbicide. It can, however, also be applied alone or together with the herbicide before or after emergence of the plants. The treatment of the plants or the seed material with the safener can therefore take place in principle independently of the time of application of the herbicide. The treatment of the plant by simultaneous application of herbicide and safener (e.g. in the form of a tank mixture) is generally preferred. The rate of application of safener relative to herbicide is largely dependent upon the mode of application. In the case of field treatment, generally from 0.001 to 5.0 kg of safener/ha, preferably from 0.001 to 0.5 kg of safener/ha, are applied. In the case of seed dressing, generally from 0.001 to 10 g of safener/kg of seed, preferably from 0.05 to 2 g of safener/kg of seed, are applied. When the safener is applied in liquid form, with seed soaking, shortly before sowing, it is advantageous to use safener solutions which contain the active ingredient in a concentration of from 1 to 10 000 ppm, preferably from 100 to 1000 ppm.

It is preferred to apply the other herbicide together with one of the safeners mentioned above. The following Examples illustrate the invention further but do not limit the invention.

### PREPARATION EXAMPLES

Those skilled in the art will appreciate that certain compounds described below are β-ketoenols, and as such may exist as a single tautomer or as a mixture of keto-enol and diketone tautomers, as described, for example by J. March, Advanced Organic Chemistry, third edition, John Wiley and Sons. The compounds shown below, and in Table T1 are drawn as an arbitrary single enol tautomer, but it should be inferred that this description covers both the diketone form and any possible enols which could arise through tautomerism. Where more than one tautomer is observed in proton NMR, the data shown are for the mixture of tautomers. Furthermore, some of the compounds shown below are drawn as single enantiomers for the purposes of simplicity, but unless specified as single enantiomers, these structures should be construed as representing a mixture of enantiomers. Additionally, some of the compounds can exist as diastereoisomers, and it should be inferred that these can be present as a mixture of diastereoisomers or as any possible single diastereoisomer. Within the detailed experimental section the diketone tautomer is chosen for naming purposes, even if the predominant tautomer is the enol form.

### Example 1

### Preparation of rac-(3aR,4R,7S,7aS)-2-(4′-chloro-4-ethylbiphenyl-3-yl)hexahydro-4,7-methanoindene-1,3-dione

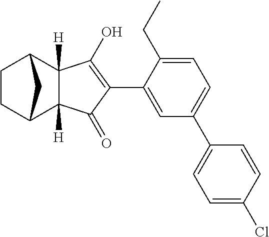

### Step 1: Preparation of rac-(3aR,4S,7R,7aS)-3a,4,7,7a-tetrahydro-4,7-methanoindene-1,3-dione

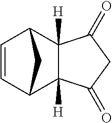

Dicyclopentadiene (20 ml) is cracked by heating to 180° C., according to known procedures (F R Hartley, Elements of Organometallic Chemistry, 1974, pages 92-94) and cyclopentadiene (approximately 10 ml), is distilled into a collecting flask containing cyclopent-4-ene-1,3-dione (3.44 g, 35.8 mmol), cooled in a salt-ice bath. The resultant reaction mixture is stirred at 0-5° C. for 2 hours, then at room temperature for 2 hours. The reaction mixture is filtered and washed with hexane to give rac-(3aR,4S,7R,7aS)-3a,4,7,7a-tetrahydro-4,7-methanoindene-1,3-dione (5.52 g), used without further purification in the next step.

### Step 2: Preparation of rac-(3aR,4R,7S,7aS)-hexahydro-4,7-methanoindene-1,3-dione

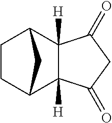

rac-(3aR,4S,7R,7aS)-3a,4,7,7a-Tetrahydro-4,7-methanoindene-1,3-dione (2.55 g, 16 mmol) is dissolved in methanol (200 ml) and hydrogenated in the presence of 5% palladium on carbon (approx. 200 mg) at 3.5 bar for 4 hours. The catalyst is removed by filtration through diatomaceous earth and the filtrate is concentrated under reduced pressure to afford rac-(3aR,4R,7S,7aS)-hexahydro-4,7-methanoindene-1,3-dione (2.29 g).

### Step 3: Preparation of rac-(3aR,4R,7S,7aS)-2-(4′-chloro-4-ethylbiphenyl-3-yl)hexahydro-4,7-methanoindene-1,3-dione

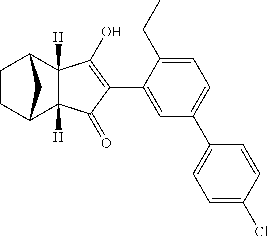

To a mixture of rac-(3aR,4R,7S,7aS)-hexahydro-4,7-methanoindene-1,3-dione (0.240 g, 1.46 mmol), N,N-dimethylaminopyridine (0.536 g, 4.39 mmol) and 4′-chloro-4-ethylbiphen-3-yllead triacetate (1.31 g, 2.18 mmol) (described in WO 2008/071405 A1) is added anhydrous chloroform (10 ml). The mixture is then heated at 40° C. for 4 hours, then stirred at room temperature for 1 hour. The mixture is diluted with ethyl acetate (200 ml) and washed with dilute aqueous hydrochloric acid (2×100 ml) then brine. The organic phase is dried over anhydrous magnesium sulfate, filtered and the filtrate is concentrated to give a yellow oil. Purification by column chromatography on silica gel (100% isohexane to 90% ethyl acetate in isohexane eluant) gives rac-(3aR,4R,7S,7aS)-2-(4′-chloro-4-ethylbiphenyl-3-yl)hexahydro-4,7-methanoindene-1,3-dione (0.328 g).

### Example 2

### Preparation of rac-(3aR,4R,7S,7aS)-2-(4′-chloro-4-ethyl-2′-fluorobiphenyl-3-yl)-hexahydro-4,7-methanoindene-1,3-dione

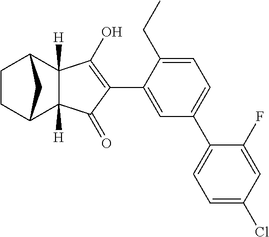

### Step 1: Preparation of (5-bromo-2-ethylphenyl)furan-2-ylmethanol

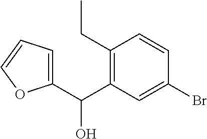

4-Bromo-2-iodoethyl benzene (50.0 g, 160.8 mmol) (described in WO 2008/071405 A1) is dissolved in anhydrous tetrahydrofuran (250 ml) and cooled to −70° C. under an atmosphere of nitrogen. Isopropylmagnesium chloride (2M solution in THF, 100 ml, 200 mmol) is added dropwise with vigorous stirring over 40 minutes, maintaining the internal temp below −60° C. by external cooling. When the addition is complete, the reaction is stirred at −70° C. for 20 minutes then allowed to warm to room temperature over 1 h 20 minutes. The reaction mixture is then cooled to −70° C. and a solution of 2-furaldehyde (16 ml, 18.6 g, 190 mmol) in tetrahydrofuran (50 ml) is added dropwise over 40 minutes. On completion of the addition, the reaction is allowed to warm to room temperature and stirred at room temperature for 3 hours. Saturated aqueous ammonium chloride solution (˜500 ml) is added and the mixture is extracted into ethyl acetate. The organic solutions are combined, washed with brine, dried over anhydrous magnesium sulfate, filtered and the filtrate is concentrated under reduced pressure. The residue is further purified by column chromatography on silica gel to give (5-bromo-2-ethylphenyl)furan-2-ylmethanol (40.7 g).

### Step 2: Preparation of 5-(5-bromo-2-ethylphenyl)-4-hydroxycyclopent-2-enone

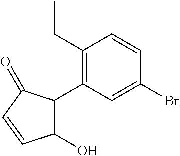

A solution of (5-bromo-2-ethylphenyl)furan-2-ylmethanol (40.73 g, 145 mmol) in acetone (1150 ml) and water (170 ml) is heated to 55° C. and 30 drops of polyphosphoric acid are added. The mixture is stirred at 55° C. for 44 hours, then cooled to room temperature. The reaction mixture is concentrated under reduced pressure to remove most of the acetone and ethyl acetate (500 ml) is added. The reaction mixture is partitioned. The aqueous phase is extracted into ethyl acetate and the organic solutions are combined, washed with saturated aqueous sodium bicarbonate solution and brine, dried over anhydrous magnesium sulfate, filtered and the filtrate is concentrated under reduced pressure. The residue is purified by column chromatography on silica gel to give 5-(5-bromo-2-ethylphenyl)-4-hydroxycyclopent-2-enone (33.67 g).

### Step 3: Preparation of 2-(5-bromo-2-ethylphenyl)cyclopent-4-ene-1,3-dione

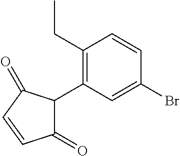

Jones' reagent (75 ml of 1.67 M solution, 125 mmol) is added dropwise over 30 minutes to a cooled (ice-bath) solution of 5-(5-bromo-4-ethylphenyl)-4-hydroxycyclopent-2-enone (33 g, 117 mmol) in acetone (400 ml). The mixture is stirred for 20 minutes, then the cooling bath is removed and the mixture is stirred for 1 hour at room temperature. Isopropanol (150 ml) is added to the yellow slurry and the mixture is stirred at room temperature for 2 hours. The mixture is diluted with ethyl acetate and washed with brine, dried over anhydrous magnesium sulfate, filtered and the filtrate is evaporated under reduced pressure to give 2-(5-bromo-2-ethylphenyl)cyclopent-4-ene-1,3-dione (32.17 g).

### Step 4: Preparation of rac-(3aR,4S,7R,7aS)-2-(5-bromo-2-ethylphenyl)-3a,4,7,7a-tetrahydro-4,7-methanoindene-1,3-dione

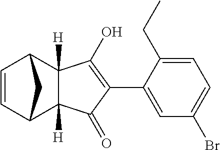

To cyclopentadiene (10 ml) (freshly cracked from dicyclopentadiene) is added 2-(5-bromo-2-ethylphenyl)-cyclopent-4-ene-1,3-dione (2.5 g, 9.24 mmol), and the mixture is stirred at 0° C. for 2 hours, then at room temperature for 18 hours.

Isohexane is added to precipitate the product, and the mixture is filtered and further washed with isohexane to afford rac-(3aR,4S,7R,7aS)-2-(5-bromo-2-ethylphenyl)-3a,4,7,7a-tetrahydro-4,7-methanoindene-1,3-dione (2.99 g) as a white solid.

### Step 5: Preparation of rac-(3aR,4R,7S,7aS)-2-(5-bromo-2-ethylphenyl)hexahydro-4,7-methanoindene-1,3-dione

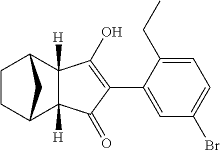

To a suspension of rac-(3aR,4S,7R,7aS)-2-(5-bromo-2-ethylphenyl)-3a,4,7,7a-tetrahydro-4,7-methanoindene-1,3-dione (1.5 g, 4.35 mmol) in methanol (200 ml) is added 5% palladium on carbon (approx. 0.20 g). The resulting suspension is stirred vigorously under a hydrogen atmosphere (3.5 bar) for 2 hours then left to stand overnight. The mixture is filtered through diatomaceous earth, washed with dichloromethane (500 ml) and the filtrate is concentrated under reduced pressure to afford rac-(3aR,4R,7S,7aS)-2-(5-bromo-2-ethylphenyl)hexahydro-4,7-methanoindene-1,3-dione as a beige solid (1.49 g).

### Step 6: Preparation of rac-(3aR,4R,7S,7aS)-2-(4′-chloro-4-ethyl-2′-fluorobiphenyl-3-yl)-hexahydro-4,7-methanoindene-1,3-dione

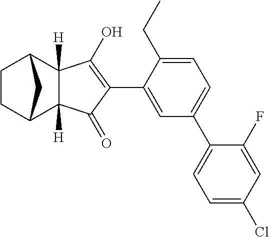

To a degassed suspension of rac-(3aR,4R,7S,7aS)-2-(5-bromo-2-ethylphenyl)hexahydro-4,7-methanoindene-1,3-dione (0.150 g, 0.43 mmol), 2-fluoro-4-chlorophenylboronic acid (0.120 g, 0.69 mmol) and cesium fluoride (0.657 g, 4.32 mmol) is added dimethoxyethane (4 ml), followed by stirring at room temperature for 40 minutes. To this mixture is then added [1,1′-bis(diphenyl-phosphino)ferrocene]-dichloropalladium(II) (0.056 g, 0.069 mmol), followed by heating at 80° C. for 18 hours. After cooling to room temperature, dichloromethane is added, and the mixture is filtered through diatomaceous earth. The filtrate is concentrated under reduced pressure, then adsorbed onto silica and purified by column chromatography on silica gel (100% isohexane to 100% ethyl acetate eluent) to afford rac-(3aR,4R,7S,7aS)-2-(4′-chloro-4-ethyl-2′-fluorobiphenyl-3-yl)hexahydro-4,7-methanoindene-1,3-dione (0.145 g) as a beige solid.

### Example 3

### Preparation of rac-(3aR,7aS)-2-(4′-chloro-4-ethyl-2′-fluorobiphenyl-3-yl)hexahydro-4,7-ethanoindene-1,3-dione

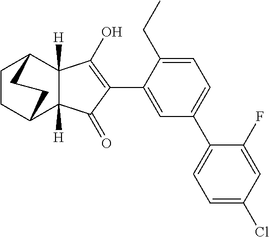

### Step 1: Preparation of rac-(3aR,4S,7R,7aS)-2-(5-bromo-2-ethylphenyl)-3a,4,7,7a-tetrahydro-4,7-ethanoindene-1,3-dione

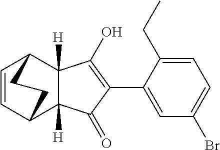

Magnesium iodide (897 mg, 3.22 mmol) is added to a solution of 2-(5-bromo-2-ethylphenyl)cyclopent-4-ene-1,3-dione (3.00 g, 10.7 mmol) in 1,3-cyclohexadiene (10 ml, 108 mmol), and the mixture is heated at 80° C. for 17 hours. The mixture is cooled to room temperature and the solvent evaporated under reduced pressure. Trituration with isohexane gives rac-(3aR,4S,7R,7aS)-2-(5-bromo-2-ethylphenyl)-3a,4,7,7a-tetrahydro-4,7-ethanoindene-1,3-dione (4.638 g) as a white solid.

### Step 2: Preparation of rac-(3aR,7aS)-2-(5-bromo-2-ethylphenyl)hexahydro-4,7-ethanoindene-1,3-dione

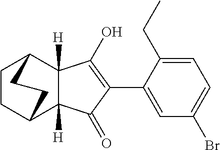

rac-(3aR,4S,7R,7aS)-2-(5-Bromo-2-ethylphenyl)-3a,4,7,7a-tetrahydro-4,7-ethanoindene-1,3-dione (3.87 g, 10.8 mmol) is dissolved in a mixture of methanol (135 ml) and ethyl acetate (45 ml) and hydrogenated over 10% palladium on carbon at 25° C. and 30 bar under continuous flow conditions (using an H-cube® supplied by ThalesNano Nanotechnology Inc. a CatCart® 10% palladium on charcoal cartridge, and a flow-rate of 1.0 ml/minute). The solvent is evaporated and the residue is purified by column chromatography on silica gel (100% isohexane to 100% ethyl acetate eluant), to give rac-(3aR,7aS)-2-(5-bromo-2-ethylphenyl)-hexahydro-4,7-ethanoindene-1,3-dione (2.484 g) as an off-white solid.

### Step 3: Preparation of rac-(3aR,7aS)-2-(4′-chloro-4-ethyl-2′-fluorobiphenyl-3-yl)hexahydro-4,7-ethanoindene-1,3-dione

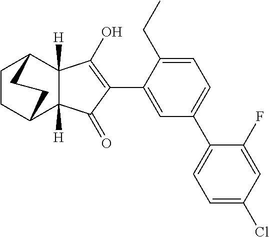

To a degassed suspension of rac-(3aR,7aS)-2-(5-bromo-2-ethylphenyl)hexahydro-4,7-ethanoindene-1,3-dione (108 mg, 0.30 mmol), 2-fluoro-4-chlorophenylboronic acid (103 mg, 0.59 mmol) and cesium fluoride (449 mg, 2.96 mmol) is added 1,2-dimethoxyethane (1.5 ml), followed by stirring at room temperature for 40 minutes. [1,1′-bis(diphenylphosphino)ferrocene]-dichloropalladium(II) (39 mg) is added and the reaction mixture is heated to 80° C. for 16 hours. The mixture is cooled to room temperature, diluted with dichloromethane and filtered through diatomaceous earth. The solvent is evaporated under reduced pressure, and the residue is purified by column chromatography on silica gel (100% isohexane to 100% ethyl acetate eluant) to give rac-(3aR,7aS)-2-(4′-chloro-4-ethyl-2′-fluorobiphenyl-3-yl)hexahydro-4,7-ethanoindene-1,3-dione (70 mg).

### Example 4

### Preparation of rac-(3aR,7aS)-2-(4′-chloro-4-ethylbiphenyl-3-yl)hexahydro-4,7-ethanoindene-1,3-dione

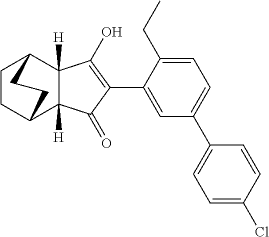

### Step 1: Preparation of rac-(3aR,4S,7R,7aS)-3a,4,7,7a-tetrahydro-4,7-ethanoindene-1,3-dione

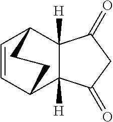

1,3-Cyclohexadiene (6.0 ml, approx. 63 mmol) and cyclopent-4-ene-1,3-dione (2.50 g, 26.0 mmol) are stirred together at room temperature for 3 days. The solid material is collected by filtration and washed with isohexane to give rac-(3aR,4S,7R,7aS)-3a,4,7,7a-tetrahydro-4,7-ethanoindene-1,3-dione (4.095 g) as a brown solid, used without further purification in the next step.

### Step 2: Preparation of rac-(3aR,7aS)-hexahydro-4,7-ethanoindene-1,3-dione

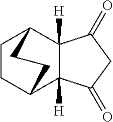

rac-(3aR,4S,7R,7aS)-3a,4,7,7a-tetrahydro-4,7-ethanoindene-1,3-dione (0.870 g, 4.94 mmol) is dissolved in methanol (200 ml) and hydrogenated in the presence of 5% palladium on carbon (approx. 85 mg) at 3.5 bar for 4 hours. The catalyst is removed by filtration through diatomaceous earth and the filtrate is concentrated under reduced pressure to afford rac-(3aR,7aS)-hexahydro-4,7-ethanoindene-1,3-dione (0.790 g).

### Step 3: Preparation of rac-(3aR,7aS)-2-(4′-chloro-4-ethylbiphenyl-3-yl)hexahydro-4,7-ethanoindene-1,3-dione

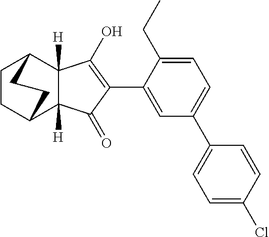

A solution of 3-bromo-4′-chloro-4-ethylbiphenyl (593 mg, 2.02 mmol) (described in WO 2008/071405 A1) in 1,4-dioxane (6 ml) is added to a mixture of rac-(3aR,7aS)-hexahydro-4,7-ethanoindene-1,3-dione (431 mg, 2.42 mmol), palladium acetate (23 mg, 10 mmol), (2-dicyclohexylphosphino)-2′,4′,6′-triisopropylbiphenyl, (72 mg, 0.15 mmol) and potassium phosphate (983 mg, 4.6 mmol) in 1,4-dioxane (6 ml). The mixture is heated at 150° C. for 45 minutes under microwave irradiation, then cooled to room temperature. The mixture is filtered through diatomaceous earth, and the filtrate is diluted with ethyl acetate and washed with 2N aqueous hydrochloric acid. The aqueous phase is extracted with ethyl acetate and the organic solutions are combined, dried over anhydrous magnesium sulfate, filtered and the filtrate is evaporated under reduced pressure. The residue is purified by column chromatography on silica gel (100% isohexane to 100% ethyl acetate eluant) to give rac-(3aR,7aS)-2-(4′-chloro-4-ethylbiphenyl-3-yl)hexahydro-4,7-ethanoindene-1,3-dione (88 mg).

### Example 5

### Preparation of rac-(3aR,4S,7R,7aS)-2-(4′-chloro-4-ethylbiphenyl-3-yl)-3a,4,7,7a-tetrahydro-4,7-ethanoindene-1,3-dione

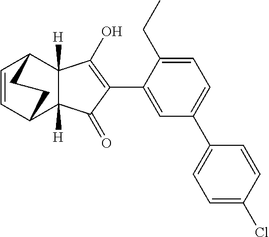

### Step 1: Preparation of (4′-chloro-4-ethylbiphen-3-yl)furan-2-ylmethanol

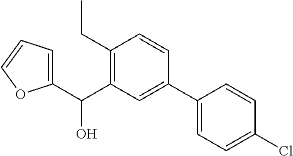

About 10 ml of a solution of 3-bromo-4′-chloro-4-ethylbiphenyl (40.0 g, 135.3 mmol) in tetrahydrofuran (200 ml) is added to magnesium turnings in a dry flask, followed by a crystal of iodine. The mixture is allowed to stand without stirring for 30 minutes, then stirred once and warmed until the orange coloured mixture becomes colourless. The remainder of the solution of 3-bromo-4′-chloro-4-ethylbiphenyl in tetrahydrofuran is added dropwise over 30 minutes with external heating applied as necessary to maintain the mixture at gentle reflux. Once the addition is complete, the mixture is heated to reflux for 2-3 hours, until only trace residues of magnesium remain. The mixture is cooled to room temperature, and then cooled further in an ice-bath. A solution of 2-furaldehyde (13.05 g, 135.8 mmol) in tetrahydrofuran (80 ml) is added dropwise over 35 minutes, and the mixture is stirred at room temperature overnight.

A second batch of material is prepared in the same way, using identical quantities of reagents and solvents, before the two batches are treated according to the procedure below.

A solution of saturated aqueous ammonium chloride (500 ml) is added to each of the mixtures prepared above, the mixtures are combined, stirred vigorously, and then allowed to stand. The two phases are separated, and the aqueous phase is extracted with ethyl acetate. The organic extracts are combined, washed with brine, dried over anhydrous magnesium sulfate, filtered and the filtrate is evaporated under reduced pressure. The residue is purified by column chromatography on silica gel to give (4′-chloro-4-ethylbiphen-3-yl)furan-2-ylmethanol (67.18 g) as a yellow oil.

### Step 2: Preparation of 5-(4′-chloro-4-ethylbiphen-3-yl)-4-hydroxycyclopent-2-enone

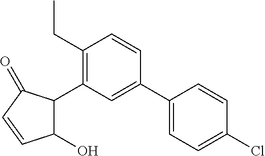

A solution of (4′-chloro-4-ethylbiphen-3-yl)furan-2-ylmethanol (67.18 g, 214.8 mmol) in acetone (1340 ml) and water (235 ml) is heated to 55° C. and 30 drops of polyphosphoric acid are added. The mixture is stirred at 55° C. for 25 hours, then cooled to room temperature. The reaction mixture is concentrated under reduced pressure to remove most of the acetone then ethyl acetate (600 ml) is added, and the reaction mixture is partitioned. The aqueous phase is extracted into ethyl acetate and the organic solutions are combined, washed with saturated aqueous sodium bicarbonate solution and brine, dried over anhydrous magnesium sulfate, filtered and the filtrate is concentrated under reduced pressure. The residue is purified by column chromatography on silica gel to give 5-(4′-chloro-4-ethylbiphen-3-yl)-4-hydroxycyclopent-2-enone (59.84 g) as a brown oil.

### Step 3: Preparation of 2-(4′-chloro-4-ethylbiphen-3-yl)cyclopent-4-ene-1,3-dione

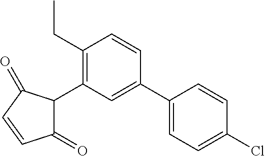

A 1.67 molar solution of Jones' reagent is prepared by adding chromium trioxide (72 g, 720 mmol) to an ice-cold mixture of concentrated sulphuric acid (72 ml) and water (360 ml) and stirring until dissolution is complete.

Jones' reagent (126 ml of 1.67 M solution, 210.4 mmol) prepared according to the procedure described above, is added dropwise over 30 minutes to a cooled (ice-bath) solution of 5-(4′-chloro-4-ethylbiphen-3-yl)-4-hydroxycyclopent-2-enone (59.84 g, 191.3 mmol) in acetone (615 ml). The mixture is stirred for 20 minutes, then the cooling bath is removed and the mixture is stirred for 1 hour at room temperature. Isopropanol (500 ml) is added to the yellow slurry and the mixture is stirred at room temperature for 2 hours. The mixture is diluted with ethyl acetate and washed with brine, dried over anhydrous magnesium sulfate, filtered and the filtrate is evaporated under reduced pressure to give 2-(4′-chloro-4-ethylbiphen-3-yl)cyclopent-4-ene-1,3-dione (47.94 g) as a yellow solid.

### Step 4: Preparation of rac-(3aR,4S,7R,7aS)-2-(4′-chloro-4-ethylbiphenyl-3-yl)-3a,4,7,7a-tetrahydro-4,7-ethanoindene-1,3-dione

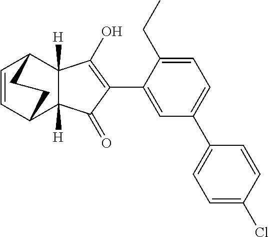

To a suspension of 2-(4′-chloro-4-ethylbiphen-3-yl)cyclopent-4-ene-1,3-dione (2.00 g, 6.44 mmol) in 1,3-cyclohexadiene (15 ml) is added magnesium iodide (0.537 g, 1.93 mmol) and the mixture was heated to 78° C. Further cyclohexadiene (10 ml) is then added, and the heating continued for 17 hours. A small amount of dichloromethane is used to rinse the condenser, added the mixture is then concentrated under reduced pressure to afford the crude product as an orange/brown solid. The residue is dissolved in a mixture of dichloromethane and methanol, absorbed onto silica, and purified by column chromatography on silica gel (100% isohexane to 100% ethyl acetate, then 10% methanol/ethyl acetate to 100% methanol as eluant) to afford rac-(3aR,4S,7R,7aS)-2-(4′-chloro-4-ethylbiphenyl-3-yl)-3a,4,7,7a-tetrahydro-4,7-ethanoindene-1,3-dione (2.104 g) as a pale yellow solid.

### Example 6

### Preparation of rac-(3aS,4R,7R,7aR)-2-(4′-chloro-4-ethylbiphenyl-3-yl)hexahydro-4,7-ethanoindene-1,3,8-trione 8-(O-methyloxime)

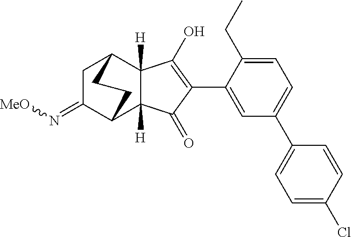

### Step 1: Preparation of rac-(3aS,4R,7R,7aR)-2-(4′-chloro-4-ethylbiphenyl-3-yl)hexahydro-4,7-ethanoindene-1,3,8-trione

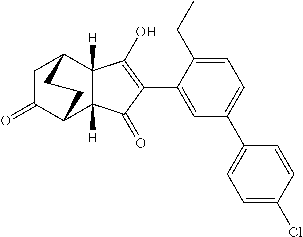

2-(4′-Chloro-4-ethylbiphen-3-yl)cyclopent-4-ene-1,3-dione (250 mg, 0.80 mmol) and magnesium iodide (67 mg, 0.24 mmol) is stirred in 2-(trimethylsilyloxy)-1,3-cyclohexadiene (3 ml, 16.1 mmol) at room temperature for 44 hours. A mixture of methanol (8 ml) and 2M aqueous hydrochloric acid (2 ml) is added and the reaction mixture is stirred at room temperature for 1 hour and 35 minutes. The solvent is evaporated under reduced pressure, the residue taken up in dichloromethane and dried over anhydrous magnesium sulfate. The mixture is filtered, and the filtrate is evaporated under reduced pressure. The residue is purified by column chromatography on silica gel (100% isohexane to 100% ethyl acetate, then to 15% methanol in ethyl acetate eluant) to give rac-(3aS,4R,7R,7aR)-2-(4′-chloro-4-ethylbiphenyl-3-yl)hexahydro-4,7-ethanoindene-1,3,8-trione (85 mg).

### Step 2: Preparation of rac-(3aS,4R,7R,7aR)-2-(4′-chloro-4-ethylbiphenyl-3-yl)hexahydro-4,7-ethanoindene-1,3,8-trione 8-(O-methyloxime)

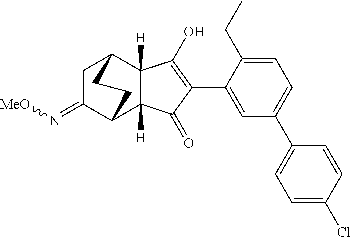

A solution of rac-(3aS,4R,7R,7aR)-2-(4′-chloro-4-ethylbiphenyl-3-yl)hexahydro-4,7-ethanoindene-1,3,8-trione (112 mg, 0.28 mmol), methoxyamine hydrochloride (69 mg, 0.83 mmol) and pyridine (68 mg, 0.83 mmol) in ethanol (10 ml) is stirred at room temperature for 4 hours. The solvent is evaporated under reduced pressure and the residue is purified by preparative reverse phase HPLC to give rac-(3aS,4R,7R,7aR)-2-(4′-chloro-4-ethylbiphenyl-3-yl)-hexahydro-4,7-ethano-indene-1,3,8-trione 8-(O-methyloxime) (60 mg) as a white solid.

### Example 7

### Preparation of rac-(3aS,4R,7R,7aS)-2-(4′-chloro-4-ethylbiphenyl-3-yl)-4-methoxy-3a,4,7,7a-tetrahydro-4,7-ethanoindene-1,3-dione

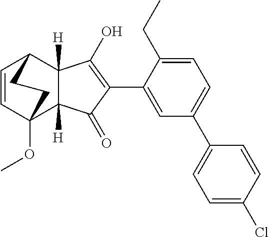

2-(4′-chloro-4-ethylbiphen-3-yl)cyclopent-4-ene-1,3-dione (250 mg, 0.80 mmol) and magnesium iodide (67 mg, 0.24 mmol) is stirred in 1-methoxy-1,3-cyclohexadiene (3 ml, 16.1 mmol) at room temperature for 21 hours. The mixture is diluted with isohexane and the resulting solid filtered, washed with isohexane and dried to give rac-(3aS,4R,7R,7aS)-2-(4′-chloro-4-ethylbiphenyl-3-yl)-4-methoxy-3a,4,7,7a-tetrahydro-4,7-ethanoindene-1,3-dione (164 mg) as a brown solid.

### Example 8

### Preparation of rac-(3aS,4R,7S,7aR)-2-(4′-chloro-4-ethylbiphenyl-3-yl)-4,5,6,7-tetramethyl-3a,4,7,7a-tetrahydro-4,7-methanoindene-1,3-dione

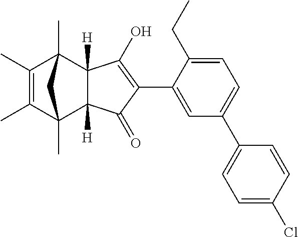

2-(4′-Chloro-4-ethylbiphenyl-3-yl)cyclopent-4-ene-1,3-dione (0.200 g, 0.64 mmol) is stirred with 1,2,3,4-tetramethylcyclopentadiene (2 ml) at room temperature for 3 days. Isohexane (2 ml) is then added, and the resulting solid is filtered and further washed with isohexane to afford rac-(3aS,4R,7S,7aR)-2-(4′-chloro-4-ethylbiphenyl-3-yl)-4,5,6,7-tetramethyl-3a,4,7,7a-tetrahydro-4,7-methanoindene-1,3-dione (0.164 g) as a white solid.

### Example 9

### Preparation of rac-(3aS,4S,7R,7aR)-2-(4′-chloro-4-ethylbiphenyl-3-yl)-8-isopropylidenehexahydro-4,7-methanoindene-1,3-dione

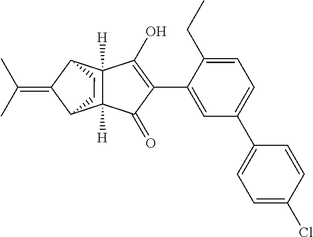

### Step 1: Preparation of rac-(4R,6S,7S,8R)-8-isopropylidenehexahydro-4,7-methanoindene-1,3-dione and rac-(4R,6S,7R,8S)-8-isopropylidenehexahydro-4,7-methanoindene-1,3-dione

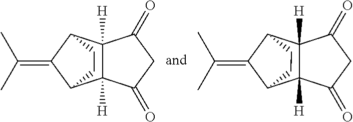

6,6-Dimethylfulvene (5 mL, 42 mmol) and 4-cyclopentenedione (2.67 g, 28 mmol) are stirred together at room temperature for 2 days, after which time the reaction mixture solidifies. The crude material is dissolved in methanol (150 ml), then 5% palladium on carbon (approx. 0.15 g) is added. The resulting suspension is stirred vigorously under a hydrogen atmosphere (3.5 bar) for 6 hours, and the suspension is then filtered through diatomaceous earth, and washed with dichloromethane. The filtrate is evaporated under reduced pressure, and the residue is purified by preparative reverse phase HPLC to afford both rac-(4R,6S,7S,8R)-8-isopropylidene-hexahydro-4,7-methanoindene-1,3-dione and rac-(4R,6S,7R,8S)-8-isopropylidenehexahydro-4,7-methanoindene-1,3-dione.

### Step 2: Preparation of rac-(3aS,4S,7R,7aR)-2-(4′-chloro-4-ethylbiphenyl-3-yl)-8-isopropylidenehexahydro-4,7-methanoindene-1,3-dione

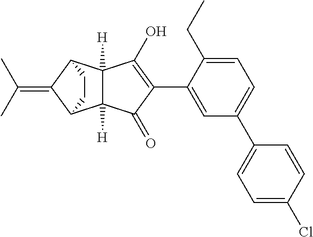

To a mixture of rac-(4R,6S,7S,8R)-8-isopropylidenehexahydro-4,7-methanoindene-1,3-dione (0.163 g, 0.80 mmol), 4′-chloro-4-ethylbiphenyl-3-yllead triacetate (0.720 g, 1.2 mmol) (described in WO 2008/071405 A1) and N,N-dimethylaminopyridine (0.293 g, 2.40 mmol) is added dry chloroform (10 ml), and the mixture is heated at 40° C. for 4.5 hours. The solution is diluted with ethyl acetate (200 ml) and washed with 1M aqueous hydrochloric acid (2×100 ml) and brine. The organic extract is dried over anhydrous magnesium sulfate, filtered and the filtrate is evapoaratured under reduced pressure. The residue is purified by column chromatography on silica gel (100% isohexane to 100% ethyl acetate eluant) to afford rac-(3aS,4S,7R,7aR)-2-(4′-chloro-4-ethylbiphenyl-3-yl)-8-isopropylidenehexahydro-4,7-methanoindene-1,3-dione (0.123 g) as a white solid.

### Example 10

### Preparation of (3aS,4S,7R,7aR)-2-(2,4,6-trimethylphenyl)hexahydro-4,7-methanoindene-1,3,8-trione 8-(O-methyloxime)

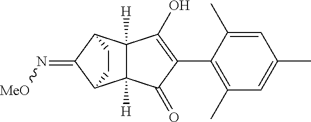

### Step 1: Preparation of (2,4,6-trimethylphenyl)furan-2-ylmethanol

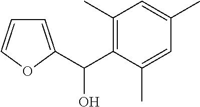

A solution of 2,4,6-trimethyl-1-bromobenzene (30.9 g, 155 mmol) in tetrahydrofuran (100 ml) is added slowly to magnesium turnings (3.77 g, 155 mmol), until the magnesium is just covered. A small quantity of iodine is added and the mixture is allowed to stand at room temperature for 25 minutes and then heated and stirred until the brown colour is lost. The remainder of the aryl bromide solution is added dropwise over a 20 minute period, with occasional heating to maintain the formation of the Grignard reagent solution. The reaction is stirred at room temperature for 1 hour. A solution of furfural (12.8 ml, 155 mmol) in tetrahydrofuran (70 ml) is added dropwise, and once the addition is complete, the reaction is stirred at room temperature for 2 hours. The reaction is quenched by cautious addition of excess saturated aqueous ammonium chloride solution, then extracted into ethyl acetate, washed with brine, dried over anhydrous magnesium sulfate, filtered and the filtrate is concentrated under reduced pressure. Purification by column chromatography on silica gel affords (2,4,6-trimethyl-phenyl)furan-2-ylmethanol.

### Step 2: Preparation of 5-(2,4,6-trimethylphenyl)-4-hydroxycyclopent-2-enone

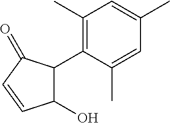

A solution of (2,4,6-trimethylphenyl)furan-2-ylmethanol (27.8 g, 129 mmol) in acetone (730 ml) and water (100 ml) is heated to 55° C. and polyphosphoric acid (2 g) is added. The mixture is stirred at 55° C. for 7 hours, then cooled to room temperature overnight. The reaction mixture is concentrated under reduced pressure to remove most of the acetone then ethyl acetate (500 ml) is added, and the reaction mixture is partitioned. The aqueous phase is extracted into ethyl acetate and the organic solutions are combined, washed with saturated aqueous sodium bicarbonate solution and brine, dried over anhydrous magnesium sulfate, filtered and the filtrate is concentrated under reduced pressure. The residue is purified by column chromatography on silica gel to give 5-(2,4,6-trimethylphenyl)-4-hydroxycyclopent-2-enone.

### Step 3: Preparation of 2-(2,4,6-trimethylphenyl)cyclopent-4-ene-1,3-dione

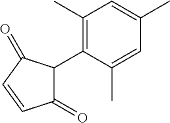

Jones' reagent (138 ml of 1.67 M solution, 230 mmol) is added dropwise over 40 minutes to a cooled (ice-bath) solution of 5-(2,4,6-trimethylphenyl)-4-hydroxycyclopent-2-enone (49.66 g, 230 mmol) in acetone (600 ml). The mixture is stirred for 1 hour. Isopropanol (100 ml) is added and the mixture is stirred at room temperature for 2 hours. The mixture is diluted with ethyl acetate and washed with brine, dried over anhydrous magnesium sulfate, filtered and the filtrate is evaporated under reduced pressure to give 2-(2,4,6-trimethylphenyl)cyclopent-4-ene-1,3-dione.

### Step 4: Preparation of rac-(3aR,4S,7R,7aS)-8-isopropylidene-2-(2,4,6-trimethylphenyl)-3a,4,7,7a-tetrahydro-4,7-methanoindene-1,3-dione and rac-(3aS,4S,7R,7aR)-8-isopropylidene-2-(2,4,6-trimethylphenyl)-3a,4,7,7a-tetrahydro-4,7-methanoindene-1,3-dione

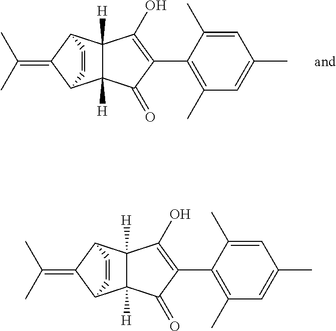

To a suspension of 2-(2,4,6-trimethylphenyl)cyclopent-4-ene-1,3-dione (5.0 g, 23.4 mmol) in toluene (40 mL) is added 6,6-dimethylfulvene (3.1 ml), and the mixture is heated at 76° C. for 18 hours. After cooling to room temperature the resulting solid is filtered, then washed with toluene to afford a 2:1 mixture of rac-(3aR,4S,7R,7aS)-8-isopropylidene-2-(2,4,6-trimethylphenyl)-3a,4,7,7a-tetrahydro-4,7-methanoindene-1,3-dione and rac-(3aS,4S,7R,7aR)-8-isopropylidene-2-(2,4,6-trimethyl-phenyl)-3a,4,7,7a-tetrahydro-4,7-methanoindene-1,3-dione (4.48 g).

### Step 5: Preparation of rac-(3aS,4S,7R,7aR)-8-isopropylidene-2-(2,4,6-trimethylphenyl)-hexahydro-4,7-methanoindene-1,3-dione

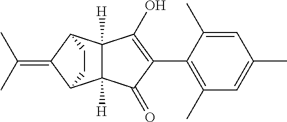

A suspension of rac-(3aR,4S,7R,7aS)-8-isopropylidene-2-(2,4,6-trimethylphenyl)-3a,4,7,7a-tetrahydro-4,7-methanoindene-1,3-dione and rac-(3aS,4S,7R,7aR)-8-Isopropylidene-2-(2,4,6-trimethyl-phenyl)-3a,4,7,7a-tetrahydro-4,7-methanoindene-1,3-dione (2.71 g, 8.5 mmol, 2:1 isomeric ratio) is warmed in methanol (500 ml) until complete dissolution. To 250 ml of this organic solution is added 5% palladium on carbon (approx. 0.20 g), and the mixture is stirred under a hydrogen atmosphere (3.5 bar) for 1.5 hours. After filtration through diatomaceous earth (washing with additional dichloromethane) the solution is then concentrated under reduced pressure. The remaining 250 ml of organic solution is hydrogenated using the same procedure for 2.5 hours, and again filtered through diatomaceous earth and the filtrate solution concentrated under reduced pressure. The organic solids are combined and recrystalised from ethyl acetate/hexane to afford rac-(3aS,4S,7R,7aR)-8-isopropylidene-2-(2,4,6-trimethylphenyl)-hexahydro-4,7-methanoindene-1,3-dione (0.51 g).

### Step 6: Preparation of rac-(3aR,4R,7S,7aR)-3-hydroxy-2-(2,4,6-trimethylphenyl)-3a,4,5,6,7,7a-hexahydro-4,7-methanoindene-1,8-dione

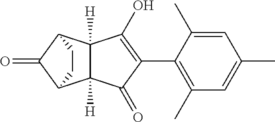

A stream of ozone is passed through a solution of rac-(3aS,4S,7R,7aR)-8-isopropylidene-2-(2,4,6-trimethylphenyl)hexahydro-4,7-methanoindene-1,3-dione (0.255 g, 0.79 mmol) in methanol (60 ml) at −78° C. over 2 hours (temperature rise to −52° C.). Oxygen is then passed through the solution for 5 minutes, then nitrogen for 15 minutes. Dimethylsulphide (0.09 ml, 1.19 mmol) is added to the reaction mixture at −42° C., and the solution is allowed to warm to room temperature and stirred under nitrogen for a total of 2.5 hours. Dichloromethane is added, and the solution is concentrated under reduced pressure. The crude product is dissolved in a mixture of dichloromethane and methanol, absorbed onto silica gel and purified by column chromatotraphy on silica gel (100% isohexane to 100% ethyl acetate eluant) to afford rac-(3aR,4R,7S,7aR)-3-hydroxy-2-(2,4,6-trimethylphenyl)-3a,4,5,6,7,7a-hexahydro-4,7-methanoindene-1,8-dione (0.050 g) as a white solid.

### Step 7: Preparation of rac-(3aS,4S,7R,7aR)-2-(2,4,6-trimethylphenyl)hexahydro-4,7-methanoindene-1,3,8-trione 8-(O-methyl oxime)

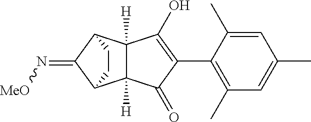

A mixture of rac-(3aS,4S,7R,7aR)-8-isopropylidene-2-(2,4,6-trimethylphenyl)-hexahydro-4,7-methanoindene-1,3-dione (0.086 g, 0.29 mmol) and methoxyamine hydrochloride (0.073 g, 0.87 mmol) in pyridine (0.07 ml) and ethanol (10 ml) is stirred at room temperature for 43 hours. The solvent is evaporated under reduced pressure, and the residue is dissolved in a mixture of dichloromethane and methanol and absorbed onto silica. Purification by column chromatography on silica gel (100% isohexane to 100% ethyl acetate eluant) affords rac-(3aS,4S,7R,7aR)-2-(2,4,6-trimethylphenyl)hexahydro-4,7-methanoindene-1,3,8-trione 8-(O-methyl oxime) (0.056 g) as a white solid.

### Example 11

### Preparation of rac-(7R,8S)-2-(2,6-diethyl-4-methylphenyl)hexahydro-4,7-ethanoindene-1,3-dione

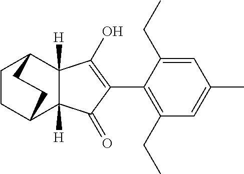

### Step 1

A suspension of iodobenzene diacetate (1.17 g, 3.65 mmol) and sodium carbonate (0.387 g, 3.65 mmol) is stirred at room temperature in distilled water (10 ml) for 20 minutes. To this mixture is then added a solution of rac-(7R,8S)-hexahydro-4,7-ethanoindene-1,3-dione (0.650 g, 3.65 mmol) and sodium carbonate (0.387 g, 3.65 mmol) in a mixture of distilled water (15 ml) and ethanol (4 ml), dropwise over 10 minutes. After stirring at room temperature for 2 hours 40 minutes the solid is filtered, washed with water and diethyl ether to afford the iodonium ylide (1.288 g) as an off-white solid.

### Step 2

To a suspension of iodonium ylide (1.28 g, 3.37 mmol) in a mixture of 1,2-dimethoxyethane (32 ml) and water (8 ml) is added 2,6-diethyl-4-methylphenylboronic acid (0.497 g, 3.70 mmol), then lithium hydroxide monohydrate (0.424 g, 10.10 mmol), tetrabutyl ammonium bromide (0.112 g, 0.34 mmol) and palladium(II) acetate (0.038 g, 0.17 mmol). The reaction mixture is heated to 50-52° C. for 4.5 hours, then cooled to room temperature. A solution of 2M aqueous hydrochloric acid (50 ml) is added. Ethyl acetate is then added, and the biphasic mixture is filtered through diatomaceous earth. The organic phase is collected, and the aqueous phase is extracted again with ethyl acetate. The organic extracts are combined, dried over magnesium sulfate, filtered and the filtrate is evaporated under reduced pressure. The residue is purified by flash column chromatography (100% hexane to 100% ethyl acetate eluant) to afford rac-(7R,8S)-2-(2,6-diethyl-4-methylphenyl)hexahydro-4,7-ethanoindene-1,3-dione (0.101 g) as a yellow solid.

### Example 12

### Preparation of rac-(5S,6S,7R)-2-(2,4,6-trimethylphenyl)hexahydro-4,7-methanoindene-1,3-dione

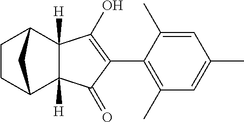

To a solution of (5S,6R,7S)-hexahydro-4,7-methanoindene-1,3-dione (0.174 g, 1.06 mmol) and N,N-dimethylaminopyridine (0.583 g, 4.77 mmol) in dry chloroform (7.5 ml) is added 2,4,6-trimethylphenyllead triacetate (0.800 g, 1.59 mmol) (described in J. Chem. Soc., Perkin 1., (1990), (3), 715-20) in one portion. The dark yellow solution is heated at 40° C. for 4 hours, then cooled to room temperature. The mixture is diluted with ethyl acetate (200 ml) and washed with 1M aqueous hydrochloric acid (2×100 ml) and brine (100 ml). The organic phase is dried over magnesium sulfate, filtered and the filtrate is evaporated under reduced pressure. The residue is purified by column chromatography on silica gel (100% isohexane to 100% ethyl acetate eluant) to afford rac-(5S,6S,7R)-2-(2,4,6-trimethylphenyl)hexahydro-4,7-methanoindene-1,3-dione (0.048 g) as a white solid.

### Example 13

### Preparation of endo-(3aR,4S,7R,7aS)-2-(3,5-dimethylbiphenyl-4-yl)-3a,4,7,7a-tetrahydro-4,7-ethanoindene-1,3-dione

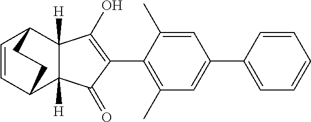

### Step 1: Preparation of 3,5-dimethylbiphen-4-ylboronic acid

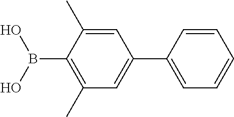

tert-Butyllithium (36.2 ml, 62.6 mmol, 1.7 M solution in hexanes) is added dropwise to a solution of 4-bromo-3,5-dimethylbiphenyl (7.27 g; 28 mmol) in dry tetrahydrofuran (150 ml) at −78° C. and stirred under an atmosphere of nitrogen for 30 minutes. Trimethylborate (9.54 ml; 84 mmol) is added and the resulting mixture is stirred at −78° C. for 30 minutes and then allowed to warm to room temperature. The reaction mixture is acidified with aqueous hydrochloric acid and extracted with ether (2×150 ml). The organic layers are combined, dried over anhydrous magnesium sulfate, filtered and the filtrate is evaporated under reduced pressure. Trituration with isohexane affords 3,5-dimethylbiphen-4-ylboronic acid (5.89 g) as a white powder.

### Step 2: Preparation of 3,5-dimethylbiphenyllead triacetate

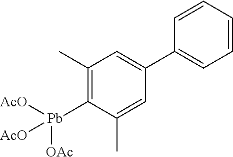

To a solution of lead tetraacetate (4.3 g; 9.7 mmol) in dry chloroform (15 ml) at 40° C. is added 3,5-dimethyl biphenyl boronic acid (2.0 g; 8.8 mmol) in one portion under a nitrogen atmosphere. Heating is continued at this temperature for 4 hours, followed by cooling to room temperature, then filtration and washing of the resulting solid with chloroform (50 ml). The filtrate is filtered through a plug of potassium carbonate/diatomaceous earth and the filtrate is concentrated under reduced pressure to afford 3,5-dimethylbiphenyllead triacetate as a brown oil (3.37 g).

### Step 3: Preparation of endo-2-(3,5-dimethylbiphenyl-4-yl)-3a,4,7,7a-tetrahydro-4,7-ethanoindene-1,3-dione

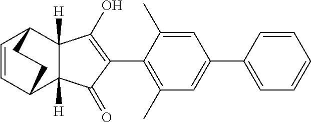

To a mixture of rac-(3aR,4S,7R,7aS)-3a,4,7,7a-tetrahydro-4,7-ethanoindene-1,3-dione (176 mg, 1 mmol) and N,N-dimethylaminopyridine (610 mg, 5 mmol) under nitrogen is added dry chloroform (5.6 ml), followed by stirring at room temperature until dissolution. To this solution is then added dry toluene (2 ml) then 3,5-dimethylbiphenyllead triacetate (0.5 M solution in dry chloroform, 2.4 ml, 1.2 mmol). The reaction mixture is then heated at reflux for 1 hour, then cooled to room temperature. A solution of 2M aqueous hydrochloric acid is added and the mixture is extracted with dichloromethane (2×40 ml). The organic fractions are combined, dried over anhydrous magnesium sulfate, filtered and the filtrate is concentrated under reduced pressure. The residue is purified by flash column chromatography to give endo-(3aR,4S,7R,7aS)-2-(3,5-dimethyl-biphenyl-4-yl)-3a,4,7,7a-tetrahydro-4,7-ethanoindene-1,3-dione (149 mg).

Additional compounds in Table T1 below were prepared by similar methods using appropriate starting materials.

TABLE T1

| Com- | | ¹H nmr (CDCl₃ |
|---|---|---|
| pound | | unless stated) or |
| Number | Structure | other physical data |
| T1 | 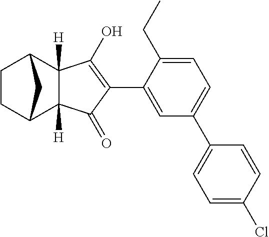 | d₄-MeOH δ 7.55 (2H, d), 7.48 (1H, dd), 7.39 (2H, d), 7.34 (1H, d), 7.19 (1H, br. s), 3.06 (2H, s), 2.63 (2H, s), 2.56 (2H, br. m), 1.79-1.76 (1H, m), 1.70-1.67 (1H, m), 1.54 (2H, d), 1.40 (2H, d), 1.14 (3H, t). |
| T2 | 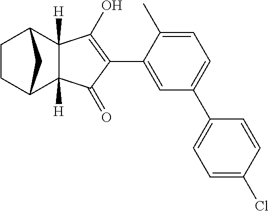 | d₄-MeOH δ 7.58-7.55 (2H, m), 7.44 (1H, dd), 7.42-7.39 (2H, m), 7.31 (1H), 7.24 (1H, s), 3.07 (2H, s), 2.65 (2H, s), 2.24 (3H, s), 1.74 (1H, d), 1.70 (1H, d), 1.55 (2H, d), 1.41 (2H, d). |
| T3 | 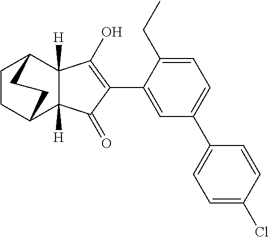 | δ 7.45-7.43 (3H, m), 7.35-7.32 (3H, m), 7.17 (1H, s), 2.69 (2H, br. s), 2.52 (2H, q), 2.11 (2H, br. s), 1.68 (2H, d), 1.59 (2H, d), 1.54 (2H, d), 1.38 (2H, d), 1.13 (3H, t). |
| T4 | 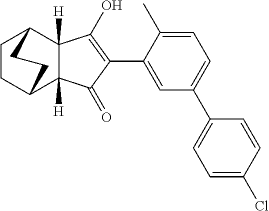 | d₄-MeOH δ 7.58-7.55 (2H, m), 7.45 (1H, dd), 7.41-7.39 (2H, m), 7.32 (1H, d), 7.27 (1H, br. s), 2.79 (2H, s), 2.24 (3H, s), 2.10 (2H, s), 1.78-1.71 (4H, m), 1.63 (2H, d), 1.44 (2H, d). |
| T5 | 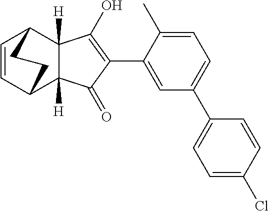 | δ 7.38-7.36 (2H, m), 7.29-7.27 (3H, m), 7.17 (1H, d), 7.10 (1H, br. s), 6.03-6.02 (2H, m), 2.94 (2H, br. s), 2.57 (2H, s), 2.07 (3H, s), 1.49 (2H, d), 1.33 (2H, d). |
| T6 | 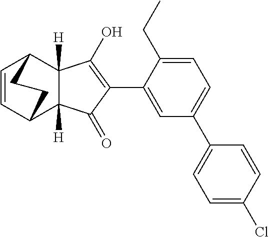 | d₄-MeOH δ 7.53 (2H, d), 7.46- 7.43 (1H, m), 7.37 (2H, d), 7.29 (1H, d), 7.15 (1H, br. s), 6.13-6.10 (2H, m), 2.99 (2H, br. s), 2.77 (2H, s), 2.46 (2H, q), 1.67 (2H, d), 1.44- 1.39 (2H, m), 1.06 (3H, t). |
| T7 | 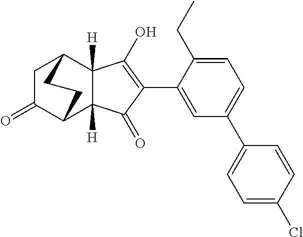 | d₆-DMSO δ 7.60 (2H, d), 7.53- 7.47 (3H, m), 7.31 (1H, d), 7.14 (1H, br. s), 2.55-2.44 (2H, m), 2.36- 2.33 (2H, m), 2.20-2.16 (2H, m), 2.00 (1H, br. s), 1.96-1.88 (2H, m), 1.84-1.75 (3H, m), 1.01 (3H, t). |
| T8 | 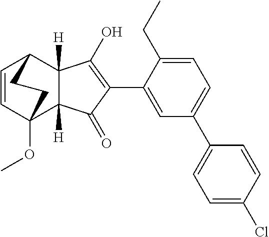 | d₆-DMSO (110° C.) δ 7.53 (2H, d), 7.42 (2H, d), 7.29 (1H, dd), 7.23 (1H, app. d), 7.17 (1H, d), 6.10 (1H, d), 6.01-5.98 (1H, m), 3.41 (3H, s), 2.86-2.54 (2H, m), 2.54 (2H, q), 2.47-2.50 (1H, m), 1.71-1.63 (2H, m), 1.56-1.52 (1H, m), 1.42- 1.38 (1H, m), 1.05 (3H, t). |
| T9 | 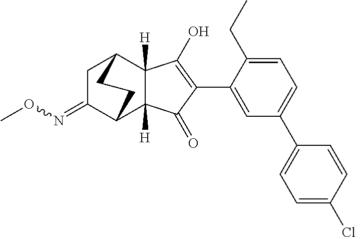 | d₆-acetone δ 7.63-7.60 (2H, m), 7.51 (1H, dd), 7.47-7.45 (2H, m), 7.34-7.31 (1H, m), 7.26-7.25 (1H, m), 3.73 and 3.67 (3H, 2 × s), 2.57-2.51 (2H, m), 2.41 (2H, br. m), 2.30-2.14 (2H, m), 1.88-1.69 (6H, m), 1.15-1.10 (3H, m). |
| T10 | 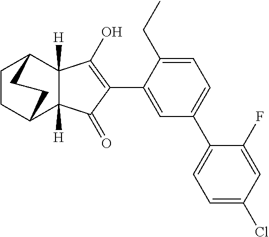 | d₄-MeOH δ 7.48-7.42 (2H, m), 7.37-7.35 (1H, m), 7.25 (2H, d), 7.15 (1H, s), 2.79 (2H, s), 2.57 (2H, q), 2.09 (2H, s), 1.79-1.68 (4H, m), 1.60 (2H, d), 1.44 (2H, d), 1.15 (3H, t). |
| T11 | 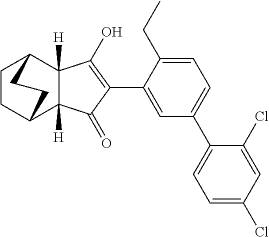 | d₄-MeOH δ 7.52 (1H, s), 7.38-7.35 (4H, m), 7.05 (1H, s), 2.78 (2H, s), 2.58 (2H, q), 2.09 (2H, s), 1.77- 1.70 (4H, m), 1.59 (2H, m), 1.44- 1.42 (2H, m), 1.16 (3H, t). |
| T12 | 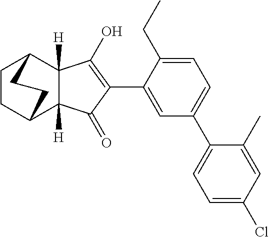 | δ 7.30-7.14 (5H, m), 6.93 (1H, s), 2.74-2.41 (4H, m), 2.44 (3H, s), 2.06 (2H, s), 1.66-1.53 (6H, m), 1.35 (2H, m), 1.16 (3H, t). |
| T13 | 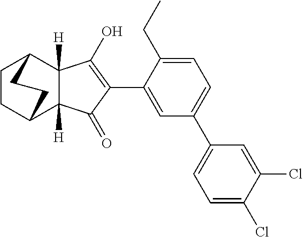 | δ 7.49 (1H, s), 7.31-7.17 (4H, m), 7.10 (1H, s), 2.72-2.62 (4H, br. m), 2.00 (2H, s), 1.58-1.44 (6H, br. m), 1.23 (2H, br. m), 1.01 (3H, t). |
| T14 | 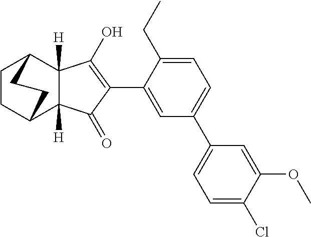 | δ 7.47-7.32 (4H, m), 7.13-7.09 (2H, m), 3.95 (3H, s), 2.70-2.62 (4H, m), 2.16 (2H, s), 1.71-1.59 (6H, m), 1.42-1.40 (2H, m), 1.19 (3H, t). |
| T15 | 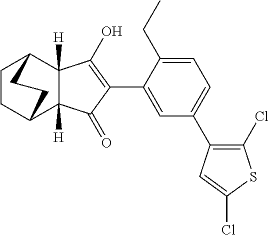 | δ 7.43 (1H, d), 7.31 (1H, d), 7.16 (1H, s), 6.87 (1H, s), 2.69-2.53 (4H, m, br), 2.12 (2H, s), 1.69- 1.54 (6H, m), 1.38-1.36 (2H, m), 1.14 (3H, t). |
| T16 | 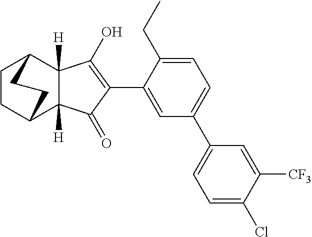 | LCMS (Method A): RT 1.88 min, M+ 460.9 |
| T17 | 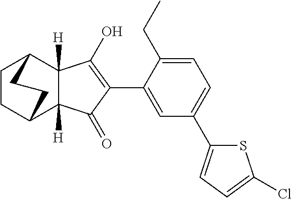 | LCMS (Method A): RT 1.79 min, M+ 398.0 |
| T18 | 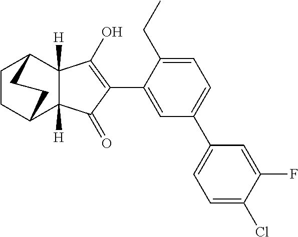 | δ 7.43-7.31 (5H, m), 7.24 (1H, s), 2.72-2.62 (4H, m), 2.17 (2H, s), 1.72-1.59 (6H, m), 1.42 (2H, br. s), 1.18 (3H, app s). |
| T19 | 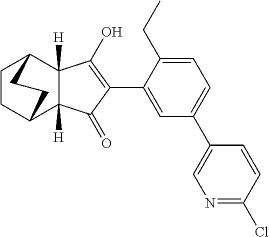 | LCMS (Method A): RT 1.55 min, M+ 393.0 |
| T20 | 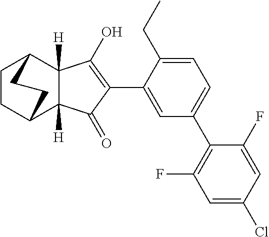 | LCMS (Method A): RT 1.80 min, M+ 428.0 |
| T21 | 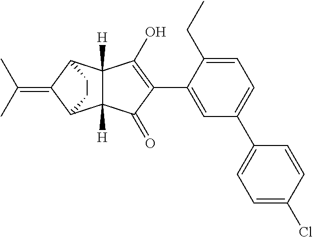 | d₄-MeOH δ 7.57-7.54 (2H, m), 7.49 (1H, dd), 7.40 (2H, d), 7.35 (1H, d), 7.20 (1H, br. s), 3.07 (2H, s), 3.01 (2H, s), 2.58 (2H, br. s), 1.72 (6H, s), 1.56-1.49 (4H, m), 1.15 (3H, t). |
| T22 | 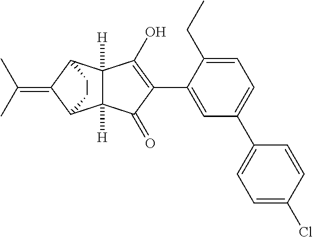 | d₄-MeOH δ 7.54 (2H, d), 7.47 (1H, dd), 7.39 (2H, d), 7.33 (1H, d), 7.17 (1H, br. s), 2.95 (2H, s), 2.63 (2H, s), 2.45 (2H, br. m), 1.73-1.71 (2H, m), 1.64 (6H, s), 1.52-1.48 (2H, m), 1.13 (3H, t). |
| T23 | 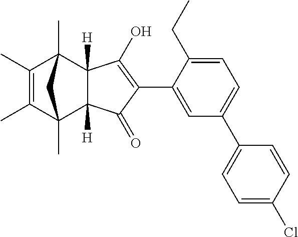 | d₆-DMSO (120° C.) δ 7.56-7.55 (2H, m), 7.48-7.45 (3H, m), 7.30 (1H, d), 7.00 (1H, s), 2.66 (1H, s), 2.43 (2H, q), 1.57 (6H, s), 1.53 (1H, d), 1.37 (6H, s), 1.34 (1H, d), 1.11 (3H, t). One proton obscured by H₂O |
| T24 | 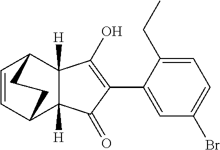 | d4-MeOH δ 7.31 (1H, dd), 7.12 (1H, d), 7.07 (1H, br. s), 6.10-6.08 (2H, m), 2.97 (2H, br. s), 2.73 (2H, s), 2.40 (2H, q), 1.69 - 1.66 (2H, m), 1.41 -1.39 (2H, m), 1.03 (3H, t). |
| T25 | 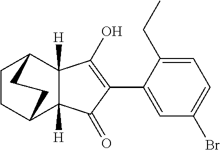 | d₄-MeOH δ 7.39 (1H, dd), 7.19 (1H, d), 7.13 (1H, br. s), 2.78 (2H, s), 2.49 (2H, q), 2.08 (2H, s), 1.79- 1.69 (4H, m), 1.57 (2H, d), 1.44 (2H, d), 1.10 (3H, t). |
| T26 | 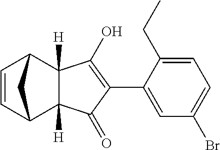 | d₄-MeOH δ 7.34 (1H, dd), 7.13 (1H, d), 7.03 (1H, br. s), 6.04 (2H, s), 3.22 (2H, br. s), 3.18 (2H, d), 2.39 (2H, q), 1.80 (1H, d), 1.68 (1H, d), 1.05 (3H, t). |
| T27 | 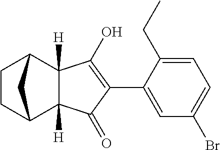 | d₄-MeOH δ 7.35 (1H, dd), 7.17 (1H, d), 7.11 (1H, s), 3.01 (2H, s), 2.62 (2H, s), 2.51-2.49 (2H, br. m), 1.76-1.74 (1H, m), 1.68-1.66 (1H, m), 1.53 (2H, br. d), 1.37 (2H, d), 1.10 (3H, t). |
| T28 | 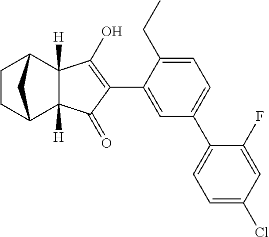 | d₄-MeOH δ 7.48-7.41 (2H, m), 7.36-7.34 (1H, m), 7.27-7.24 (2H, m), 7.13 (1H, s), 3.05 (2H, s), 2.63-2.54 (4H, m), 1.78-1.76 (1H, m), 1.70-1.67 (1H, m), 1.56- 1.51 (2H, m), 1.42-1.37 (2H, m), 1.16 (3H, t). |
| T29 | 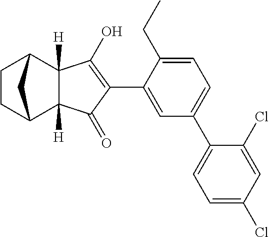 | δ 7.47 (1H, app. s), 7.35 (2H, app. s), 7.30-7.28 (2H, m), 7.08 (1H, s), 3.01 (2H, br. s), 2.65-2.59 (4H, br. m), 1.67-1.60 (2H, m), 1.49 (2H, br. s), 1.37 (2H, br. m), 1.22 (3H, t). |
| T30 | 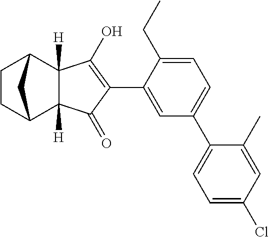 | d₆-DMSO δ 7.36 (1H, d), 7.29 (2H, d), 7.20-7.17 (2H, m), 6.86 (1H, d), 2.92 (2H, br. s), 2.52 (2H, s), 2.24 (3H, s), 1.64-1.62 (1H, d), 1.54-1.52 (1H, d), 1.41 (2H, d), 1.23 (2H, d), 1.10 (3H, t). Two protons obscured by solvent. |
| T31 | 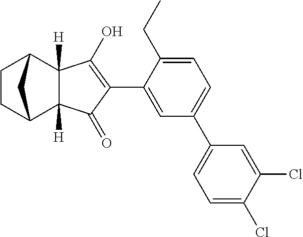 | d₆-DMSO δ 7.83 (1H, d), 7.68 (1H, m), 7.60-7.54 (2H, m), 7.33 (2H, d), 7.21 (1H, s), 2.94 (2H, br), 2.54- 2.52 (4H, m), 1.66-1.64 (1H, m), 1.55 (1H, d), 1.44 (2H, d), 1.27 (2H, d), 1.08 (3H, t). |
| T32 | 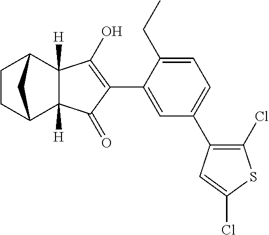 | d₆-DMSO δ 7.42 (1H, dd), 7.33- 7.31 (2H, m), 7.14 (1H, d), 2.93 (2H, br. s), 2.53 (4H, s), 1.65-1.63 (1H, m), 1.54 (1H, d), 1.43 (2H, d), 1.25 (2H, d), 1.08 (3H, t). |
| T33 | 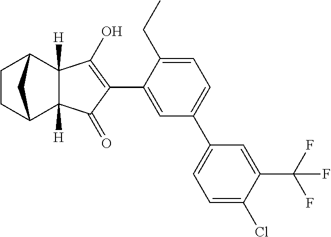 | d₆-DMSO δ 7.94 (1H, s), 7.90 (1H, d), 7.77 (1H, d), 7.59 (1H, dd), 7.35 (1H, d), 7.24 (1H, s), 2.94 (2H, s), 2.55-2.53 (4H, m), 1.66-1.63 (1H, m), 1.54 (1H, d), 1.44 (2H, d), 1.27 (2H, d), 1.08 (3H, t). |
| T34 | 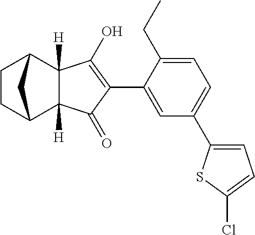 | d₆-DMSO δ 7.46 (1H, dd), 7.29- 7.26 (2H, m), 7.11 (1H, d), 7.08 (1H, s), 2.95 (2H, s), 2.55-2.53 (2H, m), 2.46-2.44 (2H, m), 1.66-1.64 (1H, m), 1.55 (1H, d), 1.44 (2H, d), 1.25 (2H, d), 1.05 (3H, t). |
| T35 | 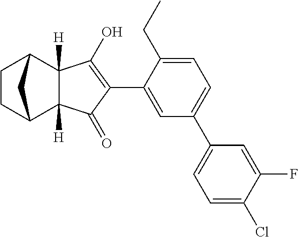 | d₆-DMSO δ 7.67-7.60 (2H, m), 7.56 (1H, dd), 7.46 (1H, dd), 7.33 (1H, d), 7.21 (1H, s), 2.95 (2H, br. s), 2.55-2.51 (4H, m), 1.65 (1H, d), 1.55 (1H, d), 1.44 (2H, d), 1.27 (2H, d), 1.08 (3H, t). |
| T36 | 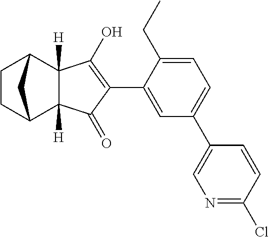 | d₆-DMSO δ 8.64 (1H, d), 8.07 (1H, dd), 7.58-7.55 (2H, m), 7.35 (1H, d), 7.23 (1H, s), 2.93 (2H, s), 2.54- 2.53 (2H, m), 1.66-1.63 (1H, m), 1.54 (1H, d), 1.43 (2H, d), 1.28 (2H, d), 1.08 (3H, t). Two protons obscured by solvent. |
| T37 | 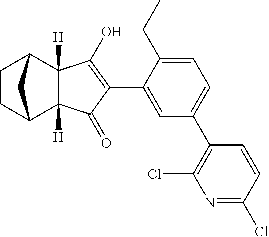 | d₆-DMSO δ 7.89 (1H, d), 7.63 (1H, d), 7.38-7.34 (2H, m), 7.04 (1H, s), 2.94 (2H, br. s), 2.54-2.52 (4H, m), 1.64 (1H, d), 1.54 (1H, d), 1.43- 1.41 (2H, m), 1.24 (2H, d), 1.10 (3H, t). |
| T38 | 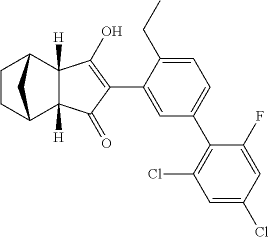 | d₄-MeOH δ 7.44 (1H, s), 7.39 (1H, d), 7.30 (1H, dd), 7.24 (1H, d), 6.97 (1H, s), 3.06 (2H, s), 1.78-1.75 (1H, m), 1.69-1.67 (1H, m), 1.52 (2H, br. s), 1.38 (2H, br. s), 1.18 (3H, t, J7.3). Four protons obscured by solvent. |
| T39 | 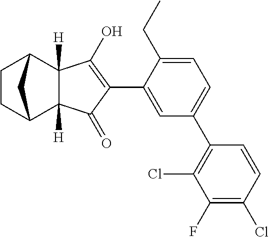 | d₆-DMSO δ 7.64 (1H, t), 7.36-7.32 (2H, m), 7.26 (1H, d), 7.01 (1H, s), 2.94 (2H, br. s), 2.53 (4H, m), 1.64 (1H, d), 1.54 (1H, d), 1.42 (2H, br. d), 1.23 (2H, d), 1.10 (3H, t). |
| T40 | 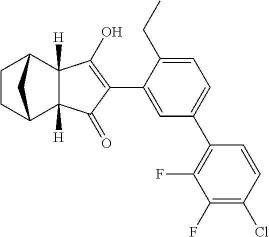 | d₆-DMSO δ 7.51-7.47 (1H, m), 7.44-7.42 (1H, m), 7.37-7.32 (2H, m), 7.11 (1H, s), 2.95 (2H, br. s), 2.53-2.49 (4H, m), 1.65-1.63 (1H, m), 1.54 (1H, d), 1.43 (2H, d), 1.25 (2H, d), 1.09 (3H, t). |
| T41 | 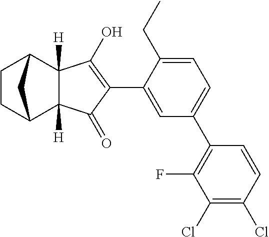 | d₆-DMSO δ 7.59-7.56 (1H, m), 7.48 (1H, t), 7.43-7.41 (1H, m), 7.37-7.35 (1H, m), 7.10 (1H, s), 2.95 (2H, br. s), 2.54-2.51 (4H, m), 1.65-1.63 (1H, m), 1.54 (1H, d), 1.43 (2H, d), 1.25 (2H, d), 1.09 (3H, t). |
| T42 | 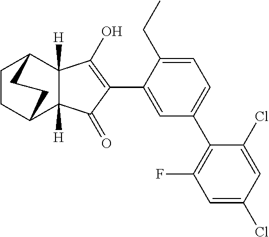 | δ 7.37 (1H, d), 7.29-7.28 (1H, m), 7.27 (1H, br. s), 7.08 (1H, dd), 6.99 (1H, s), 2.71 (2H, br. s), 2.58 (2H, q), 2.12 (2H, br. s), 1.69-1.59 (4H, m), 1.54 (2H, d), 1.37 (2H, d), 1.17 (3H, t). |
| T43 | 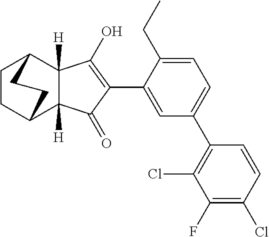 | d₆-DMSO δ 7.66-7.62 (1H, m), 7.36-7.32 (2H, m), 7.27 (1H, dd), 7.03 (1H, s), 2.65 (2H, br. s), 2.55- 2.52 (2H, m), 2.02 (2H, br. s), 1.64- 1.58 (4H, m), 1.44 (2H, d), 1.32 (2H, d), 1.10 (3H, t). |
| T44 | 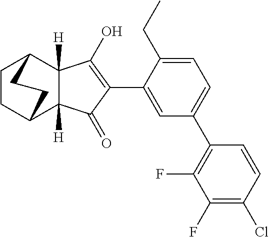 | d₆-DMSO δ 7.51-7.47 (1H, m), 7.45-7.42 (1H, m), 7.38-7.33 (2H, m), 7.13 (1H, s), 2.67 (2H, br. s), 2.54-2.50 (2H, m), 2.03 (2H, s), 1.65-1.58 (4H, m), 1.45 (2H, d), 1.33 (2H, d), 1.08 (3H, t). |
| T45 | 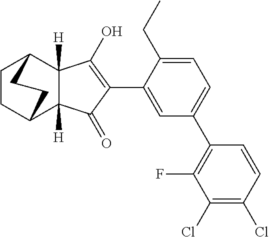 | d₆-DMSO δ 7.58 (1H, dd), 7.51- 7.47 (1H, m), 7.42-7.40 (1H, m), 7.35 (1H, d), 7.13 (1H, s), 2.66- 2.63 (2H, m), 2.55-2.53 (2H, m), 2.01 (2H, s), 1.64-1.58 (4H, m), 1.45 (2H, d), 1.32 (2H, d), 1.08 (3H, t). |
| T46 | 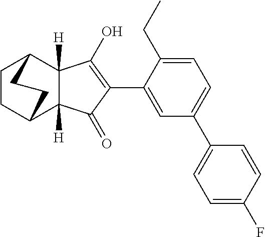 | d₆-DMSO δ 7.63-7.60 (2H, m), 7.47 (1H, dd), 7.31-7.23 (3H, m), 7.18-7.17 (1H, m), 2.66-2.61 (2H, m), 2.02 (2H, br. s), 1.65- 1.58 (4H, m), 1.47 (2H, d), 1.32 (2H, d), 1.07 (3H, t). Two protons obscured by solvent. |
| T47 | 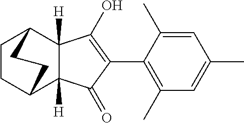 | δ 6.86 (2H, s), 2.08 (2H, br. s), 2.25 (3H, s), 2.10 (3H, s), 2.07 (2H, s), 2.03 (3H, s), 1.66 (2H, d), 1.58- 1.53 (4H, m), 1.35 (2H, d). |
| T48 | 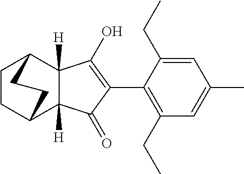 | δ 6.93 (2H, s), 2.70 (2H, br. s), 2.41 (2H, q), 2.63-2.32 (2H, m), 2.30 (3H, s), 2.11 (2H, br. s), 1.67 (2H, br. d), 1.58-1.55 (4H, br. m), 1.38 (2H, d), 1.09 (3H, t), 1.04 (3H, t). |
| T49 | 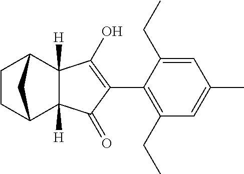 | δ 6.90 (2H, d), 2.85 (2H, s), 2.54 (2H, s), 2.44 (2H, q), 2.33-2.28 (5H, m), 1.56 (2H, s), 1.46 (2H, br. 1.37 (2H, br. d), 1.11 (3H, t), 1.04 (3H, t). |
| T50 | 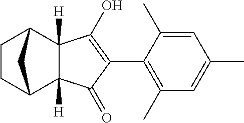 | d₄-MeOH δ 6.86 (2H, s), 3.06 (2H, s), 2.62 (2H, s), 2.24 (3H, s), 2.16 (3H, s), 2.02 (3H, s), 1.79-1.77 (1H, m), 1.70-1.68 (1H, m), 1.55- 1.52 (2H, m), 1.45-1.43 (2H, m). |
| T51 | 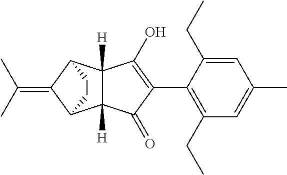 | d₄-MeOH δ 6.91 (2H, d), 3.07 (2H, s), 3.01 (2H, s), 2.47 (2H, q), 2.33 (2H, q), 2.29 (3H, s), 1.72 (6H, s), 1.54 (4H, s), 1.12 (3H, t), 1.03 (3H, t). |
| T52 | 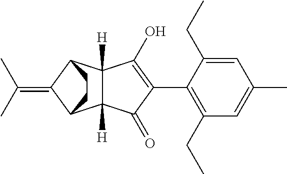 | d₄-MeOH δ 6.88 (2H, d), 2.95 (2H, d), 2.62 (2H, s), 2.35-2.26 (7H, m), 1.70-1.64 (8H, m), 1.48 (2H, d), 1.09 (3H, t), 1.04-1.01 (3H, m). |
| T53 | 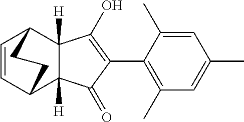 | δ 6.83 (2H, d), 6.04 (2H, dd), 2.94 (2H, s), 2.56 (2H, s), 2.23 (3H, s), 2.03 (3H, s), 2.02 (3H, s), 1.52 (2H, d), 1.35 (2H, d). |
| T54 | 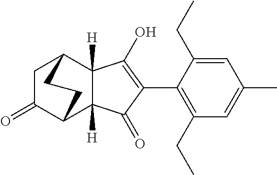 | LCMS (Method B): RT 1.40 min, MH+ 339 |
| T55 | 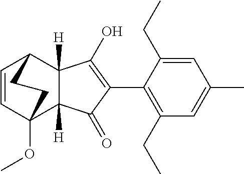 | δ (2H, s), 6.24-6.22 (1H, m), 6.19-6.15 (1H, m), 3.49 (3H, s), 3.12-3.08 (1H, br. m), 2.98 (1H, d), 2.78 (1H, dd), 2.37-2.27 (7H, m), 1.91-1.84 (1H, m), 1.79-1.73 (1H, m), 1.60-1.49 (2H, m), 1.06- 0.99 (6H, m). |
| T56 | 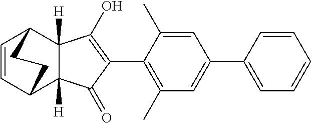 | d₄-MeOH δ 7.55 (m, 2H), 7.39 (m, 2H), 7.29 (m, 1H), 7.27 (s, 2H), 6.14 (m, 2H), 3.00 (br s, 2H), 2.85 (s, 2H), 2.14 (s, 3H), 2.12 (s, 3H), 1.70 (m, 2H), 1.44 (m, 2H). |
| T57 | 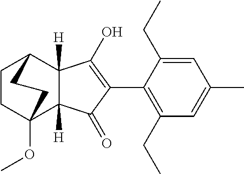 | δ 8.03 (1H, s), 6.92 (2H, s), 3.29 (3H, s), 3.16 (1H, d), 2.96 (1H, dd), 2.85-2.82 (1H, m), 2.49-2.42 (2H, m), 2.41-2.34 (2H, m), 2.30 (3H, s), 1.97-1.75 (4H, m), 1.64- 1.51 (4H, m), 1.13 (3H, t), 1.07 (3H, t). |
| T58 | 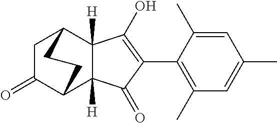 | LCMS (Method B): RT 1.22 min, MH+ 311 |
| T59 | 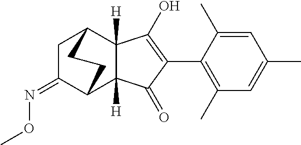 | d₆-acetone δ 6.79 (2H, s), 3.78 (1H, d), 3.64 (3H, s), 2.99 (1H, br. s), 2.39 (1H, s), 2.30 (1H, d), 2.21 (3H, s), 2.15 (1H, d), 2.07 (3H, s), 2.01 (3H, s), 1.85-1.72 (4H, m). One proton obscured by H₂O. |
| T60 | 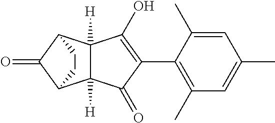 | d₄-MeOH δ 6.85 (2H, d), 3.01 (2H, s), 2.24-2.22 (5H, m), 2.06-2.03 (5H, m), 1.91 (3H, s), 1.84-1.80 (2H, m). |
| T61 | 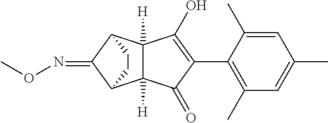 | δ 6.86 (2H, d), 3.70 (3H, s), 3.38 (1H, d), 2.74 (1H, d), 2.64 (2H, s), 2.24 (3H, s), 2.04 (3H, s), 2.02 (3H, s), 1.88-1.86 (2H, m), 1.51 (2H, d). |

It should be noted that certain compounds of the invention exist as a mixture of isomers noted above, under the conditions used to obtain the ¹H NMR data. Where this has occurred, the characterising data are reported for all isomers present at ambient temperature in the specified solvent. Unless otherwise stated, proton NMR spectra were recorded at ambient temperature. Compounds characterised by HPLC-MS were analysed using one of two methods described below.

Method A

Compounds characterised by HPLC-MS were analysed using an Waters 2777 injector, 2996 photodiode array, 2420 ELSD and Micromass ZQ2000 equipped with a Waters Atlantis dC18 column (column length 20 mm, internal diameter of column 3 mm, particle size 3 micron). The analysis was conducted using a three minute run time, according to the following gradient table:

| Time | Solvent A | Solvent B | Flow |
|---|---|---|---|
| (mins) | (%) | (%) | (ml/mn) |
| 0.00 | 95.0 | 5.0 | 1.300 |
| 2.50 | 0.0 | 100 | 1.300 |
| 2.80 | 0.00 | 100 | 1.300 |
| 2.90 | 95.0 | 5.0 | 1.300 |

Solvent A: H₂O with 0.05% TFA
Solvent B: CH₃CN with 0.05% TFA

The characteristic values obtained for each compound were the retention time (recorded in minutes) and the molecular ion, typically the cation M+H⁺ as listed in table T1.

Method B

Compounds characterised by HPLC-MS were analysed using a Waters 2795 HPLC equipped with a Waters Atlantis dC18 column (column length 20 mm, internal diameter of column 3 mm, particle size 3 micron, temperature 40° C.), Waters photodiode array and Micromass ZQ2000. The analysis was conducted using a three minute run time, according to the following gradient table:

| Time | Solvent A | Solvent B | Flow |
|---|---|---|---|
| (mins) | (%) | (%) | (ml/mn) |
| 0.00 | 90.0 | 10.0 | 2.00 |
| 0.25 | 90.0 | 100 | 2.00 |
| 2.00 | 10.0 | 90.0 | 2.00 |
| 2.50 | 10.0 | 90.0 | 2.00 |
| 2.60 | 90.0 | 10.0 | 2.00 |
| 3.0 | 90.0 | 10.0 | 2.00 |

Solvent A: H₂O containing 0.1% HCOOH
Solvent B: CH₃CN containing 0.1% HCOOH

The characteristic values obtained for each compound were the retention time (rt, recorded in minutes) and the molecular ion (typically the cation MH⁺), as listed in Table T1.

The compounds of the following Tables 1 to 24 can be obtained in an analogous manner.

TABLE 1
This table covers 252 compounds of the formula I
(I)

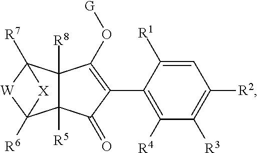

wherein R¹ is methyl, R⁴, R⁵, R⁶, R⁷ and R⁸ are hydrogen,
X is —CH₂—, W is —CH═CH—,
G is hydrogen and R² and R³ are as defined below:

| Com- | | |
|---|---|---|
| pound | | |
| Number | R² | R³ |
| 1.001 | phenyl | H |
| 1.002 | 2-fluorophenyl | H |
| 1.003 | 3-fluorophenyl | H |
| 1.004 | 4-fluorophenyl | H |
| 1.005 | 2-chlorophenyl | H |
| 1.006 | 3-chlorophenyl | H |
| 1.007 | 4-chlorophenyl | H |
| 1.008 | 2-bromophenyl | H |
| 1.009 | 3-bromophenyl | H |
| 1.010 | 4-bromophenyl | H |
| 1.011 | 4-tert-butylphenyl | H |
| 1.012 | 2-iodophenyl | H |
| 1.013 | 3-iodophenyl | H |
| 1.014 | 4-iodophenyl | H |
| 1.015 | 2-methylphenyl | H |
| 1.016 | 3-methylphenyl | H |
| 1.017 | 4-methylphenyl | H |
| 1.018 | 2-cyanophenyl | H |
| 1.019 | 3-cyanophenyl | H |
| 1.020 | 4-cyanophenyl | H |
| 1.021 | 2-methoxyphenyl | H |
| 1.022 | 3-methoxyphenyl | H |
| 1.023 | 4-methoxyphenyl | H |
| 1.024 | 2-difluoromethoxyphenyl | H |
| 1.025 | 3-difluoromethoxyphenyl | H |
| 1.026 | 4-difluoromethoxyphenyl | H |
| 1.027 | 2-difluoromethylphenyl | H |
| 1.028 | 3-difluoromethylphenyl | H |
| 1.029 | 4-difluoromethylphenyl | H |
| 1.030 | 2-trifluoromethylphenyl | H |
| 1.031 | 3-trifluoromethylphenyl | H |
| 1.032 | 4-trifluoromethylphenyl | H |
| 1.033 | 2-trifluoromethoxyphenyl | H |
| 1.034 | 3-trifluoromethoxyphenyl | H |
| 1.035 | 4-trifluoromethoxyphenyl | H |
| 1.036 | 4-methylthiophenyl | H |
| 1.037 | 4-methylsulfinylphenyl | |
| 1.038 | 4-methylsulfonylphenyl | H |
| 1.039 | 4-trifluoromethylthiophenyl | H |
| 1.040 | 4- | H |
| | trifluoromethylsulfinylphenyl | |
| 1.041 | 4- | H |
| | trifluoromethylsulfonylphenyl | |
| 1.042 | 2,3-difluorophenyl | H |
| 1.043 | 2,4-difluorophenyl | H |
| 1.044 | 2,5-difluorophenyl | H |
| 1.045 | 2,6-difluorophenyl | H |
| 1.046 | 3,4-difluorophenyl | H |
| 1.047 | 3,5-difluorophenyl | H |
| 1.048 | 2,3-dichlorophenyl | |
| 1.049 | 2,4-dichlorophenyl | H |
| 1.050 | 2,5-dichlorophenyl | H |
| 1.051 | 2,6-dichlorophenyl | H |
| 1.052 | 3,4-dichlorophenyl | H |
| 1.053 | 3,5-dichlorophenyl | H |
| 1.054 | 4-chloro-2-cyanophenyl | H |
| 1.055 | 4-chloro-3-cyanophenyl | H |
| 1.056 | 4-chloro-2-fluorophenyl | H |
| 1.057 | 4-chloro-3-fluorophenyl | H |
| 1.058 | 4-chloro-2-methoxyphenyl | H |
| 1.059 | 4-chloro-3-methoxyphenyl | H |
| 1.060 | 4-chloro-2-methylphenyl | H |
| 1.061 | 4-chloro-3-methylphenyl | H |
| 1.062 | 4-chloro-2- | H |
| | difluoromethoxyphenyl | |
| 1.063 | 4-chloro-3- | H |
| | difluoromethoxyphenyl | |
| 1.064 | 4-chloro-2- | H |
| | trifluoromethoxyphenyl | |
| 1.065 | 4-chloro-3- | H |
| | trifluoromethoxyphenyl | |
| 1.066 | 4-chloro-2- | H |
| | difluoromethylphenyl | |
| 1.067 | 4-chloro-3- | H |
| | difluoromethylphenyl | |
| 1.068 | 4-chloro-2- | H |
| | trifluoromethylphenyl | |
| 1.069 | 4-chloro-3- | H |
| | trifluoromethylphenyl | |
| 1.070 | 4-chloro-2,3-difluorophenyl | H |
| 1.071 | 4-chloro-2,5-difluorophenyl | H |
| 1.072 | 4-chloro-2,6-difluorophenyl | H |
| 1.073 | 2,4-dichloro-3-fluorophenyl | H |
| 1.074 | 2,4-dichloro-5-fluorophenyl | H |
| 1.075 | 2,4-dichloro-6-fluorophenyl | H |
| 1.076 | 2,3,4-trichlorophenyl | H |
| 1.077 | 2,3,5-trichlorophenyl | H |
| 1.078 | 2,3,6-trichlorophenyl | H |
| 1.079 | 2,4,5-trichlorophenyl | H |
| 1.080 | 2,4,6-trichlorophenyl | H |
| 1.081 | 2,3,4-trifluorophenyl | H |
| 1.082 | 2,3,5-trifluorophenyl | H |
| 1.083 | 2,3,6-trifluorophenyl | H |
| 1.084 | 2,4,5-trifluorophenyl | H |
| 1.085 | 2,4,6-trifluorophenyl | H |
| 1.086 | 2-fluoro-4- | H |
| | trifluoromethylphenyl | |
| 1.087 | 3-fluoro-4- | H |
| | trifluoromethylphenyl | |
| 1.088 | 2-chloropyridin-5-yl | H |
| 1.089 | 3-chloropyridinyl-5-yl | H |
| 1.090 | 2-methylpyridin-5-yl | H |
| 1.091 | 3-methylpyridinyl-5-yl | H |
| 1.092 | 2-trifluoromethylpyridin-5-yl | H |
| 1.093 | 3-trifluoromethylpyridin-5-yl | H |
| 1.094 | 2-chloro-3-methylpyridin-5-yl | H |
| 1.095 | 2-chloro-4-methylpyridin-5-yl | H |
| 1.096 | 6-chloro-2-methylpyridin-3-yl | H |
| 1.097 | 2,3-dichloropyridin-5-yl | H |
| 1.098 | 2,4-dichloropyridin-5-yl | H |
| 1.099 | 2,6-dichloropyridin-3-yl | H |
| 1.100 | pyrazin-2-yl | H |
| 1.101 | 2-chloropyrazin-5-yl | H |
| 1.102 | 2-bromopyrazin-5-yl | H |
| 1.103 | pyridazin-3-yl | H |
| 1.104 | 6-bromopyridazin-3-yl | H |
| 1.105 | 6-chloropyridazin-3-yl | H |
| 1.106 | pyrimidin-5-yl | H |
| 1.107 | 2-bromopyrimidin-5-yl | H |
| 1.108 | 5-bromopyrimidin-2-yl | H |
| 1.109 | 2-chloropyrimidin-5-yl | H |
| 1.110 | 5-chloropyrimidin-2-yl | H |
| 1.111 | 2-furyl | H |
| 1.112 | 3-furyl | H |
| 1.113 | 2-thienyl | H |
| 1.114 | 3-thienyl | H |
| 1.115 | 4-bromothien-2-yl | H |
| 1.116 | 5-bromothien-2-yl | H |
| 1.117 | 4-chlorothien-2-yl | H |
| 1.118 | 5-chlorothien-2-yl | H |
| 1.119 | pyrazol-1-yl | H |
| 1.120 | 3-chloropyrazol-1-yl | H |
| 1.121 | 4-chloropyrazol-1-yl | H |
| 1.122 | 1-methylpyrazol-4-yl | H |
| 1.123 | 1-methyl-3- | H |
| | trifluoromethylpyrazol-5-yl | |
| 1.124 | 2-thiazolyl | H |
| 1.125 | 4-methylthiazol-2-yl | H |
| 1.126 | 5-methylthiazol-2-yl | H |
| 1.127 | H | phenyl |
| 1.128 | H | 2-fluorophenyl |
| 1.129 | H | 3-fluorophenyl |
| 1.130 | H | 4-fluorophenyl |
| 1.131 | H | 2-chlorophenyl |
| 1.132 | H | 3-chlorophenyl |
| 1.133 | H | 4-chlorophenyl |
| 1.134 | H | 2-bromophenyl |
| 1.135 | H | 3-bromophenyl |
| 1.136 | H | 4-bromophenyl |
| 1.137 | H | 4-tert-butylphenyl |
| 1.138 | H | 2-iodophenyl |
| 1.139 | H | 3-iodophenyl |
| 1.140 | H | 4-iodophenyl |
| 1.141 | H | 2-methylphenyl |
| 1.142 | H | 3-methylphenyl |
| 1.143 | H | 4-methylphenyl |
| 1.144 | H | 2-cyanophenyl |
| 1.145 | H | 3-cyanophenyl |
| 1.146 | H | 4-cyanophenyl |
| 1.147 | H | 2-methoxyphenyl |
| 1.148 | H | 3-methoxyphenyl |
| 1.149 | H | 4-methoxyphenyl |
| 1.150 | H | 2-difluoromethoxyphenyl |
| 1.151 | H | 3-difluoromethoxyphenyl |
| 1.156 | H | 4-difluoromethoxyphenyl |
| 1.157 | H | 2-difluoromethylphenyl |
| 1.158 | H | 3-difluoromethylphenyl |
| 1.159 | H | 4-difluoromethylphenyl |
| 1.160 | H | 2-trifluoromethyiphenyl |
| 1.161 | H | 3-trifluoromethyiphenyl |
| 1.162 | H | 4-trifluoromethyiphenyl |
| 1.163 | H | 2-trifluoromethoxyphenyl |
| 1.164 | H | 3-trifluoromethoxyphenyl |
| 1.165 | H | 4-trifluoromethoxyphenyl |
| 1.166 | H | 4-methylthiophenyl |
| 1.167 | H | 4-methylsulfinylphenyl |
| 1.168 | H | 4-methylsulfonylphenyl |
| 1.169 | H | 4-trifluoromethylthiophenyl |
| 1.170 | H | 4- |
| | | trifluoromethylsulfinylphenyl |
| 1.171 | H | 4- |
| | | trifluoromethylsulfonylphenyl |
| 1.172 | H | 2,3-difluorophenyl |
| 1.173 | H | 2,4-difluorophenyl |
| 1.174 | H | 2,5-difluorophenyl |
| 1.175 | H | 2,6-difluorophenyl |
| 1.176 | H | 3,4-difluorophenyl |
| 1.177 | H | 3,5-difluorophenyl |
| 1.178 | H | 2,3-dichlorophenyl |
| 1.179 | H | 2,4-dichlorophenyl |
| 1.180 | H | 2,5-dichlorophenyl |
| 1.181 | H | 2,6-dichlorophenyl |
| 1.182 | H | 3,4-dichlorophenyl |
| 1.183 | H | 3,5-dichlorophenyl |
| 1.184 | H | 4-chloro-2-cyanophenyl |
| 1.185 | H | 4-chloro-3-cyanophenyl |
| 1.186 | H | 4-chloro-2-fluorophenyl |
| 1.187 | H | 4-chloro-3-fluorophenyl |
| 1.188 | H | 4-chloro-2-methoxyphenyl |
| 1.189 | H | 4-chloro-3-methoxyphenyl |
| 1.190 | H | 4-chloro-2-methylphenyl |
| 1.191 | H | 4-chloro-3-methylphenyl |
| 1.192 | H | 4-chloro-2- |
| | | difluoromethoxyphenyl |
| 1.193 | H | 4-chloro-3- |
| | | difluoromethoxyphenyl |
| 1.194 | H | 4-chloro-2- |
| | | trifluoromethoxyphenyl |
| 1.195 | H | 4-chloro-3- |
| | | trifluoromethoxyphenyl |
| 1.196 | H | 4-chloro-2- |
| | | difluoromethylphenyl |
| 1.197 | H | 4-chloro-3- |
| | | difluoromethylphenyl |
| 1.198 | H | 4-chloro-2- |
| | | trifluoromethylphenyl |
| 1.199 | H | 4-chloro-3- |
| | | trifluoromethylphenyl |
| 1.200 | H | 4-chloro-2,3-difluorophenyl |
| 1.201 | H | 4-chloro-2,5-difluorophenyl |
| 1.202 | H | 4-chloro-2,6-difluorophenyl |
| 1.203 | H | 2,4-dichloro-3-fluorophenyl |
| 1.204 | H | 2,4-dichloro-5-fluorophenyl |
| 1.205 | H | 2,4-dichloro-6-fluorophenyl |
| 1.206 | H | 2,3,4-trichlorophenyl |
| 1.207 | H | 2,3,5-trichlorophenyl |
| 1.208 | H | 2,3,6-trichlorophenyl |
| 1.209 | H | 2,4,5-trichlorophenyl |
| 1.210 | H | 2,4,6-trichlorophenyl |
| 1.211 | H | 2,3,4-trifluorophenyl |
| 1.212 | H | 2,3,5-trifluorophenyl |
| 1.213 | H | 2,3,6-trifluorophenyl |
| 1.214 | H | 2,4,5-trifluorophenyl |
| 1.215 | H | 2,4,6-trifluorophenyl |
| 1.216 | H | 2-fluoro-4- |
| | | trifluoromethylphenyl |
| 1.217 | H | 3-fluoro-4- |
| | | trifluoromethylphenyl |
| 1.218 | H | 2-chloropyridin-5-yl |
| 1.219 | H | 3-chloropyridinyl-5-yl |
| 1.220 | H | 2-methylpyridin-5-yl |
| 1.221 | H | 3-methylpyridinyl-5-yl |
| 1.222 | H | 2-trifluoromethylpyridin-5-yl |
| 1.223 | H | 3-trifluoromethylpyridin-5-yl |
| 1.224 | H | 2-chloro-3-methylpyridin-5-yl |
| 1.225 | H | 2-chloro-4-methylpyridin-5-yl |
| 1.226 | H | 6-chloro-2-methylpyridin-3-yl |
| 1.227 | H | 2,3-dichloropyridin-5-yl |
| 1.228 | H | 2,4-dichloropyridin-5-yl |
| 1.229 | H | 2,6-dichloropyridin-3-yl |
| 1.230 | H | pyrazin-2-yl |
| 1.231 | H | 2-chloropyrazin-5-yl |
| 1.232 | H | 2-bromopyrazin-5-yl |
| 1.233 | H | pyridazin-3-yl |
| 1.234 | H | 6-bromopyridazin-3-yl |
| 1.235 | H | 6-chloropyridazin-3-yl |
| 1.236 | H | pyrimidin-5-yl |
| 1.237 | H | 2-bromopyrimidin-5-yl |
| 1.238 | H | 5-bromopyrimidin-2-yl |
| 1.239 | H | 2-chloropyrimidin-5-yl |
| 1.240 | H | 5-chloropyrimidin-2-yl |
| 1.241 | H | 2-furyl |
| 1.242 | H | 3-furyl |
| 1.243 | H | 2-thienyl |
| 1.244 | H | 3-thienyl |
| 1.245 | H | 4-bromothien-2-yl |
| 1.246 | H | 5-bromothien-2-yl |
| 1.247 | H | 4-chlorothien-2-yl |
| 1.248 | H | 5-chlorothien-2-yl |
| 1.249 | H | pyrazol-1-yl |
| 1.250 | H | 3-chloropyrazol-1-yl |
| 1.251 | H | 4-chloropyrazol-1-yl |
| 1.252 | H | 1-methylpyrazol-4-yl |
| 1.253 | H | 1-methyl-3- |
| | | trifluoromethylpyrazol-5-yl |
| 1.254 | H | 2-thiazolyl |
| 1.255 | H | 4-methylthiazol-2-yl |
| 1.256 | H | 5-methylthiazol-2-yl |

Table 2

This table covers 252 compounds of the formula I, wherein R¹ is methyl, R⁴, R⁵, R⁶, R⁷ and R⁸ are hydrogen, X is —CH₂—, W is —CH₂CH₂—, G is hydrogen and

R² and R³ are as defined in Table 1.

Table 3

This table covers 252 compounds of the formula I, wherein

R¹ is ethyl, R⁴, R⁵, R⁶, R⁷ and R⁸ are hydrogen, X is —CH₂—, W is —CH═CH—, G is hydrogen and R² and R³ are as defined below:

Table 4

This table covers 252 compounds of the formula I, wherein

R¹ is ethyl, R⁴, R⁵, R⁶, R⁷ and R⁸ are hydrogen, X is —CH₂—, W is —CH₂CH₂—, G is hydrogen and R² and R³ are as defined in Table 1.

Table 5

This table covers 252 compounds of the formula I, wherein

R¹ is chlorine, R⁴, R⁵, R⁶, R⁷ and R⁸ are hydrogen, X is —CH₂—, W is —CH═CH—, G is hydrogen and R² and R³ are as defined below:

Table 6

This table covers 252 compounds of the formula I, wherein

R¹ is chlorine, R⁴, R⁵, R⁶, R⁷ and R⁸ are hydrogen, X is —CH₂—, W is —CH₂CH₂—, G is hydrogen and

R² and R³ are as defined in Table 1.

Table 7

This table covers 252 compounds of the formula I, wherein

R¹ and R⁴ are methyl, R⁵, R⁶, R⁷ and R⁸ are hydrogen, X is —CH₂—, W is —CH═CH—, G is hydrogen and R² and R³ are as defined below:

Table 8

This table covers 252 compounds of the formula I, wherein

R¹ and R⁴ are methyl, R⁵, R⁶, R⁷ and R⁸ are hydrogen, X is —CH₂—, W is —CH₂CH₂—, G is hydrogen and R² and R³ are as defined in Table 1.

Table 9

This table covers 252 compounds of the formula I, wherein

R¹ is methyl, R⁴ is ethyl, R⁵, R⁶, R⁷ and R⁸ are hydrogen, X is —CH₂—, W is —CH═CH—, G is hydrogen and R² and R³ are as defined below:

Table 10

This table covers 252 compounds of the formula I, wherein

R¹ is methyl, R⁴ is ethyl, R⁵, R⁶, R⁷ and R⁸ are hydrogen, X is —CH₂—, W is —CH₂CH₂—, G is hydrogen and R² and R³ are as defined in Table 1.

Table 11

This table covers 252 compounds of the formula I, wherein

R¹ and R⁴ are ethyl, R⁵, R⁶, R⁷ and R⁸ are hydrogen, X is —CH₂—, W is —CH═CH—, G is hydrogen and R² and R³ are as defined below:

Table 12

This table covers 252 compounds of the formula I, wherein

R¹ and R⁴ are ethyl, R⁵, R⁶, R⁷ and R⁸ are hydrogen, X is —CH₂—, W is —CH₂CH₂—, G is hydrogen and R² and R³ are as defined in Table 1.

Table 13

This table covers 252 compounds of the formula I, wherein

R¹ is methyl, R⁴, R⁵, R⁶, R⁷ and R⁸ are hydrogen, X is —CH₂CH₂—, W is —CH═CH—, G is hydrogen and R² and R³ are as in Table 1

Table 14

This table covers 252 compounds of the formula I, wherein

R¹ is methyl, R⁴, R⁵, R⁶, R⁷ and R⁸ are hydrogen, X is —CH₂CH₂—, W is —CH₂CH₂—, G is hydrogen and R² and R³ are as defined in Table 1.

Table 15

This table covers 252 compounds of the formula I, wherein

R¹ is ethyl, R⁴, R⁵, R⁶, R⁷ and R⁸ are hydrogen, X is —CH₂CH₂—, W is —CH═CH—, G is hydrogen and R² and R³ are as defined below:

Table 16

This table covers 252 compounds of the formula I, wherein

R¹ is ethyl, R⁴, R⁵, R⁶, R⁷ and R⁸ are hydrogen, X—CH₂CH₂—, W is —CH₂CH₂—, G is hydrogen and R² and R³ are as defined in Table 1.

Table 17

This table covers 252 compounds of the formula I, wherein

R¹ is chlorine, R⁴, R⁵, R⁶, R⁷ and R⁸ are hydrogen, X is —CH₂CH₂—, W is —CH═CH—, G is hydrogen and R² and R³ are as defined below:

Table 18

This table covers 252 compounds of the formula I, wherein

R¹ is chlorine, R⁴, R⁵, R⁶, R⁷ and R⁸ are hydrogen, X is —CH₂CH₂—, W is —CH₂CH₂—, G is hydrogen and R² and R³ are as defined in Table 1.

Table 19

This table covers 252 compounds of the formula I, wherein

R¹ and R⁴ are methyl, R⁵, R⁶, R⁷ and R⁸ are hydrogen, X is —CH₂CH₂—, W is —CH═CH—, G is hydrogen and R² and R³ are as defined below:

Table 20

This table covers 252 compounds of the formula I, wherein

R¹ and R⁴ are methyl, R⁵, R⁶, R⁷ and R⁸ are hydrogen, X is —CH₂CH₂—, W is —CH₂CH₂—, G is hydrogen and R² and R³ are as defined in Table 1.

Table 21

This table covers 252 compounds of the formula I, wherein

R¹ is methyl, R⁴ is ethyl, R⁵, R⁶, R⁷ and R⁸ are hydrogen, X is —CH₂CH₂—, W is —CH═CH—, G is hydrogen and R² and R³ are as defined below:

Table 22

This table covers 252 compounds of the formula I, wherein

R¹ is methyl, R⁴ is ethyl, R⁵, R⁶, R⁷ and R⁸ are hydrogen, X is —CH₂CH₂—, W is —CH₂CH₂—, G is hydrogen and R² and R³ are as defined in Table 1.

Table 23

This table covers 252 compounds of the formula I, wherein

R¹ and R⁴ are ethyl, R⁵, R⁸, R⁷ and R⁸ are hydrogen, X is —CH₂CH₂—, W is —CH═CH—, G is hydrogen and R² and R³ are as defined below:

Table 24

This table covers 252 compounds of the formula I, wherein

R¹ and R⁴ are ethyl, R⁵, R⁸, R⁷ and R⁸ are hydrogen, X is —CH₂CH₂—, W is —CH₂CH₂—, G is hydrogen and R² and R³ are as defined in Table 1.

### BIOLOGICAL EXAMPLES

### Test Example 1

Monocotyledonous and dicotyledonous test plants were sown in standard soil in pots. After cultivation for one day (pre-emergence) or after 10 days cultivation (post-emergence) under controlled conditions in a glasshouse, the plants were sprayed with an aqueous spray solution derived from the formulation of the technical active ingredient in 0.6 ml acetone and 45 ml formulation solution containing 10.6% Emulsogen EL (Registry number 61791-12-6), 42.2% N-methylpyrrolidone, 42.2% dipropylene glycol monomethyl ether (Registry number 34590-94-8) and 0.2% X-77 (Registry number 11097-66-8). The test plants were then grown in a greenhouse under optimum conditions until, 15 days later for post-emergence and 20 days for pre-emergence, the test was evaluated (100=total damage to plant; 0=no damage to plant).

Test Plants:

Lolium perenne (LOLPE), Setaria faberi (SETFA), Digitaria sanguinalis (DIGSA), Alopecurus myosuroides (ALOMY), Echinochloa crus-galli (ECHCG) and Avena fatua (AVEFA),

Pre-Emergence Data:

| Compound | Rate | | | | | | |
|---|---|---|---|---|---|---|---|
| Number | g/ha | LOLPE | SEFTA | DIGSA | ALOMY | ECHCG | AVEFA |
| T8 | 250 | 70 | 90 | 100 | 60 | 100 | 10 |
| T21 | 250 | 40 | 60 | 50 | 60 | 30 | 20 |
| T47 | 250 | 80 | 100 | 100 | 90 | 70 | 50 |
| T48 | 250 | 100 | 100 | 100 | 100 | 100 | 70 |
| T49 | 250 | 100 | 100 | 100 | 100 | 100 | 70 |
| T50 | 250 | 100 | 100 | 80 | 70 | 70 | 50 |
| T51 | 250 | 100 | 80 | 80 | 60 | 90 | 60 |
| T52 | 250 | 70 | 100 | 80 | 50 | 90 | 60 |
| T53 | 250 | 100 | 100 | 80 | 70 | 70 | 40 |
| T54 | 250 | 90 | 80 | 80 | 70 | 70 | 70 |
| T55 | 250 | 100 | 100 | 100 | 100 | 100 | 70 |

Post-Emergence Data:

| Compound | Rate | | | | | | |
|---|---|---|---|---|---|---|---|
| Number | g/ha | LOLPE | SEFTA | DIGSA | ALOMY | ECHCG | AVEFA |
| T8 | 125 | 0 | 80 | 100 | 70 | 100 | 10 |
| T21 | 125 | 0 | 20 | 20 | 20 | 0 | 20 |
| T47 | 125 | 40 | 60 | 80 | 0 | 60 | 40 |
| T48 | 125 | 100 | 80 | 100 | 80 | 90 | 70 |
| T49 | 125 | 100 | 100 | 100 | 100 | 100 | 80 |
| T50 | 125 | 30 | 0 | 30 | 30 | 20 | 10 |
| T51 | 125 | 50 | 0 | 30 | 30 | 10 | 0 |
| T52 | 125 | 50 | 80 | 70 | 90 | 80 | 20 |
| T53 | 125 | 30 | 20 | 50 | 20 | 60 | 0 |
| T54 | 125 | 50 | 80 | 80 | 60 | 80 | 60 |
| T55 | 125 | 70 | 90 | 80 | 80 | 100 | 80 |

### Test Example B

Seeds of a variety of test species were sown in standard soil in pots. After cultivation for one day (pre-emergence) or after 8 days cultivation (post-emergence) under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). The test plants were then grown in a glasshouse under controlled conditions in a glasshouse (at 24/16° C., day/night; 14 hours light; 65% humidity) and watered twice daily. After 13 days for pre and post-emergence, the test was evaluated (100=total damage to plant; 0=no damage to plant).

Test Plants:

Setaria faberi (SETFA), Alopecurus myosuroides (ALOMY), Echinochloa crus-galli (ECHCG), and Avena fatua (AVEFA).

Pre-Emergence Data:

| Compound | Rate | | | | |
|---|---|---|---|---|---|
| Number | g/ha | SETFA | ALOMY | ECHCG | AVEFA |
| T1 | 250 | — | 70 | 100 | 100 |
| T2 | 250 | — | 30 | 100 | 80 |
| T3 | 250 | — | 70 | 100 | 70 |
| T4 | 250 | — | 50 | 100 | 70 |
| T6 | 250 | — | 60 | 100 | 40 |
| T7 | 250 | 90 | 90 | 100 | 70 |
| T9 | 250 | 0 | 0 | 0 | 0 |
| T10 | 250 | — | 0 | 90 | 0 |
| T11 | 250 | — | 0 | 60 | 0 |
| T12 | 250 | — | 60 | 100 | 20 |
| T13 | 250 | — | 20 | 90 | 0 |
| T14 | 250 | — | 80 | 100 | 70 |
| T15 | 250 | — | 0 | 30 | 0 |
| T16 | 250 | — | 60 | 100 | 20 |
| T17 | 250 | — | 80 | 100 | 90 |
| T18 | 250 | — | 50 | 90 | 50 |
| T20 | 250 | — | 100 | 100 | 100 |
| T28 | 250 | — | 60 | 100 | 90 |
| T29 | 250 | — | 30 | 100 | 90 |
| T30 | 250 | — | 40 | 100 | 30 |
| T31 | 250 | — | 20 | 80 | 70 |
| T33 | 250 | — | 20 | 100 | 30 |
| T34 | 250 | — | 30 | 100 | 40 |
| T35 | 250 | — | 80 | 90 | 70 |
| T36 | 250 | — | 30 | 80 | 40 |
| T37 | 250 | — | 50 | 90 | 70 |
| T38 | 250 | — | 0 | 80 | 0 |
| T39 | 250 | — | 40 | 100 | 70 |
| T40 | 250 | — | 50 | 100 | 60 |
| T41 | 250 | — | 0 | 50 | 0 |
| T42 | 250 | — | 0 | 0 | 0 |
| T43 | 250 | — | 0 | 40 | 20 |
| T44 | 250 | — | 0 | 70 | 20 |
| T46 | 250 | — | 20 | 80 | 0 |
| T56 | 250 | 100 | 100 | 100 | 20 |
| T57 | 250 | 100 | 100 | 100 | 100 |
| T59 | 250 | 80 | 90 | 80 | 30 |
| T62 | 250 | 20 | 0 | 20 | 30 |

Post-Emergence Data:

| Compound | Rate | | | | |
|---|---|---|---|---|---|
| Number | g/ha | SETFA | ALOMY | ECHCG | AVEFA |
| T1 | 250 | — | 90 | 100 | 100 |
| T2 | 250 | — | 40 | 100 | 60 |
| T3 | 250 | — | 100 | 100 | 90 |
| T4 | 250 | — | 70 | 100 | 80 |
| T6 | 250 | — | 70 | 100 | 60 |
| T7 | 250 | 60 | 20 | 20 | 20 |
| T9 | 250 | 100 | 70 | 90 | 60 |
| T10 | 250 | — | 0 | 90 | 0 |
| T11 | 250 | — | 90 | 100 | 100 |
| T12 | 250 | — | 90 | 100 | 40 |
| T13 | 250 | — | 60 | 100 | 60 |
| T14 | 250 | — | 0 | 90 | 0 |
| T15 | 250 | — | 40 | 80 | 30 |
| T16 | 250 | — | 0 | 30 | 0 |
| T17 | 250 | — | 40 | 90 | 50 |
| T18 | 250 | — | 100 | 100 | 100 |
| T20 | 250 | — | 100 | 100 | 100 |
| T28 | 250 | — | 30 | 90 | 10 |
| T29 | 250 | — | 30 | 100 | 0 |
| T30 | 250 | — | 0 | 90 | 0 |
| T31 | 250 | — | 60 | 100 | 70 |
| T33 | 250 | — | 70 | 90 | 70 |
| T34 | 250 | — | 60 | 90 | 80 |
| T35 | 250 | — | 90 | 90 | 90 |
| T36 | 250 | — | 0 | 50 | 20 |
| T37 | 250 | — | 70 | 90 | 0 |
| T38 | 250 | — | 30 | 90 | 40 |
| T39 | 250 | — | 70 | 100 | 50 |
| T40 | 250 | — | 90 | 100 | 90 |
| T41 | 250 | — | 0 | 90 | 60 |
| T42 | 250 | — | 0 | 80 | 40 |
| T43 | 250 | — | 70 | 100 | 30 |
| T44 | 250 | — | 90 | 100 | 80 |
| T46 | 250 | — | 50 | 100 | 30 |
| T56 | 250 | 100 | 60 | 100 | 60 |
| T57 | 250 | 100 | 70 | 100 | 30 |
| T59 | 250 | 80 | 30 | 50 | 0 |
| T62 | 250 | 80 | 50 | 60 | 0 |

What is claimed is:

1. Compounds of formula I 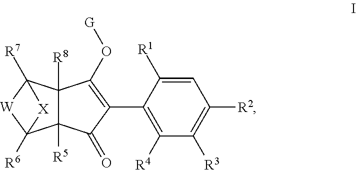
wherein
R¹ is methyl, ethyl, n-propyl, isopropyl, cyclopropyl, halomethyl, haloethyl, vinyl, ethynyl, halogen, methoxy, ethoxy, halomethoxy or haloethoxy,
R² and R³ are independently of each other hydrogen, methyl, ethyl, n-propyl, isopropyl, cyclopropyl, halomethyl, haloethyl, vinyl, propenyl, ethynyl, propynyl, halogen, methoxy, ethoxy, halomethoxy or haloethoxy, optionally substituted aryl or optionally substituted heteroaryl,
R⁴ is hydrogen, methyl, ethyl, n-propyl, isopropyl, cyclopropyl, halomethyl, haloethyl, vinyl, propenyl, ethynyl, propynyl, halogen, methoxy, ethoxy, halomethoxy or haloethoxy,
R⁵ and R⁸ are independently of each other hydrogen, C₁-C₃alkyl, C₁-C₃haloalkyl, C₁-C₃alkoxy, C₁-C₃alkylthio, halogen or C₁-C₆alkoxycarbonyl, or
R⁵ and R⁸ join together to form a 3-7 membered carbocyclic or heterocyclic ring containing an oxygen or sulfur atom,
R⁶ and R⁷ are independently of each other hydrogen, halogen, cyano, hydroxy, optionally substituted C₁-C₆alkyl, optionally substituted C₂-C₆alkenyl, optionally substituted C₂-C₇alkynyl, optionally substituted C₁-C₆alkoxy or tri-C₁-C₄alkylsilyloxy,
X is optionally substituted C₁-C₃alkylene,
W is optionally substituted C₁-C₃alkylene or optionally substituted C₂-C₃alkenylene and
G is hydrogen or an agriculturally acceptable metal, sulfonium, ammonium or latentiating group.

2. Compounds according to claim 1, wherein R¹ is methyl, ethyl, n-propyl, cyclopropyl, halomethyl, haloethyl, halogen, vinyl or ethynyl.

3. Compounds according to claim 1, wherein R² and R³ are independently of each other hydrogen, phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃haloalkoxy, cyano, nitro or halogen.

4. Compounds according to claim 1, wherein R⁴ is hydrogen, methyl or ethyl.

5. Compounds according to claim 1, wherein R¹ is ethyl, R² is hydrogen, R³ is phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃haloalkoxy, cyano, nitro or halogen, and R⁴ is hydrogen.

6. Compounds according to claim 1, wherein R¹ is methyl or ethyl, R² is phenyl or phenyl substituted by C₁-C₄alkyl, C₁-C₃haloalkyl, C₁-C₃alkoxy, C₁-C₃haloalkoxy, cyano, nitro or halogen, or heteroaryl or heteroaryl substituted by C₁-C₄alkyl, C₁-C₃haloalkyl, C₁-C₃alkoxy, C₁-C₃haloalkoxy, cyano, nitro or halogen, R³ is hydrogen and R⁴ is hydrogen, methyl or ethyl.

7. Compounds according to claim 1, wherein R¹ is methyl or ethyl, R² is methyl, R³ is hydrogen and R⁴ is methyl or ethyl.

8. Compounds according to claim 1, wherein R¹ is ethyl, R² is hydrogen, R³ is phenyl substituted in the para-position by chlorine, bromine or iodine, and optionally further substituted once or twice by C₁-C₄alkyl, C₁-C₃haloalkyl, C₁-C₃alkoxy, C₁-C₃haloalkoxy, cyano, nitro or halogen, and R⁴ is hydrogen.

9. Compounds according to claim 1, wherein R⁵ and R⁸ are independently of each other hydrogen or methyl.

10. Compounds according to claim 1, wherein R⁶ and R⁷ are independently of each other hydrogen, optionally substituted C₁-C₆alkyl, optionally substituted C₂-C₆alkenyl, optionally substituted C₁-C₆alkoxy or tri-C₁-C₄alkylsilyloxy.

11. Compounds according to claim 1, wherein X is optionally substituted methylene or ethylene.

12. Compounds according to claim 1, wherein W is —CR⁹═CR¹⁰— or —CHR⁹—CHR¹⁰—, wherein R⁹ and R¹⁰ are independently of each other hydrogen, optionally substituted C₁-C₆alkyl, optionally substituted C₂-C₆alkenyl, optionally substituted C₁-C₆alkoxy or tri-C₁-C₄alkylsilyloxy, or is a fragment —CH₂—C(O)— or —CH₂—C(═NOR¹¹)—, wherein R¹¹ is C₁-C₆alkyl.

13. A process for the preparation of a compound of formula I according to claim 1 as herein described.

14. Compounds of the formula (AE) and (S) 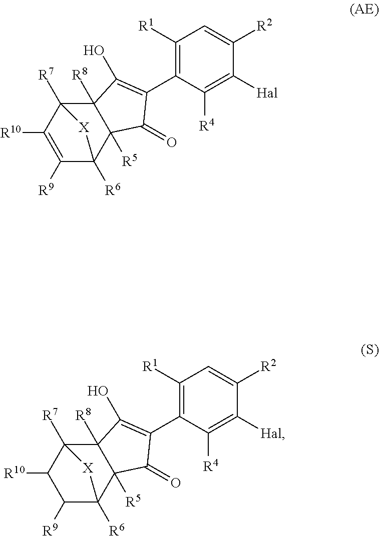
wherein R¹, R², R⁴R⁵, R⁶, R⁷, R⁸ and X have the meanings assigned to them in claim 1, R⁹ and R¹⁰ are independently of each other hydrogen, optionally substituted C₁-C₆alkyl, optionally substituted C₂-C₆alkenyl, optionally substituted C₁-C₆alkoxy or tri-C₁-C₄alkylsilyloxy, and Hal is chlorine, bromine or iodine.

15. Compounds of the formula (AK) and (W) 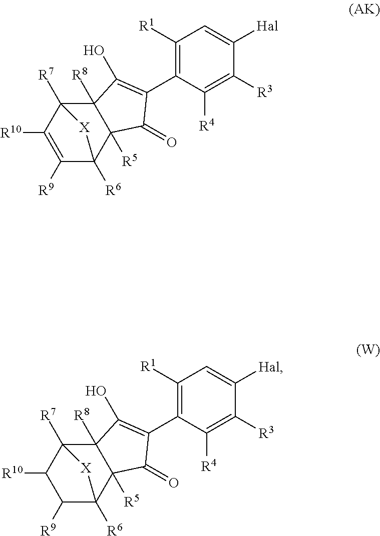
wherein R¹, R³, R⁴R⁵, R⁶, R⁷, R⁸ and X have the meanings assigned to them in claim 1, R⁹ and R¹⁰ are independently of each other hydrogen, optionally substituted C₁-C₆alkyl, optionally substituted C₂-C₆alkenyl, optionally substituted C₁-C₆alkoxy or tri-C₁-C₄alkylsilyloxy, and Hal is chlorine, bromine or iodine.

16. A herbicidal composition, which, in addition to comprising formulation assistants, comprises a herbicidally effective amount of a compound of formula I according to claim 1, optionally a further herbicide as mixing partner, optionally a safener and optionally an adjuvant.

17. A method of controlling grasses and weeds in crops of useful plants, which comprises applying a herbicidally effective amount of a compound of formula I according to claim 1, or of a composition comprising such a compound, to the plants or to the locus thereof.
